SEQUENCE LISTING (2 sequences)

SEQ ID NO: 1 - DNA, 60 bp, Artificial Sequence
  Synthetic polynucleotide
aatgatacggcgaccaccgagatctacactatggtaattgtgtgccagcmgccgcggtaa

SEQ ID NO: 2 - DNA, 56 bp, Artificial Sequence
  Synthetic polynucleotide
  misc_feature: A 12-bp Golay barcode is between positions 24 and 25
caagcagaagacggcatacgagatagtcagtcagccggactachvgggtwtctaat

(19) United States
(12) Patent Application Publication
(10) Pub. No.: US 2020/0197389 A1
(43) Pub. Date: Jun. 25, 2020

(54) METHODS FOR DETECTING DYSBIOSIS AND TREATING SUBJECTS WITH DYSBIOSIS

(71) Applicant: THE REGENTS OF THE UNIVERSITY OF CALIFORNIA, Oakland, CA (US)

(72) Inventor: Susan LYNCH, Piedmont, CA (US)

(21) Appl. No.: 16/609,118

(22) PCT Filed: May 11, 2018
(86) PCT No.: PCT/US18/32436

§ 371 Date: Oct. 28, 2019

Related U.S. Application Data
(60) Provisional application No. 62/505,637, filed on May 12, 2017.

(51) Int. Cl. A61K 31/496; C12Q 1/04; C12Q 1/689; C12Q 1/6895; A61K 31/4409; A61K 45/06; A61P 31/06; A61P 31/10

(52) U.S. Cl. CPC A61K 31/496; C12Q 1/04; C12Q 1/689; A61P 31/10; A61K 31/4409; A61K 45/06; A61P 31/06; C12Q 1/6895

(57) ABSTRACT

Provided herein are, inter alia, methods, compositions, and systems, for detecting and treating lung dysbiosis. In aspects, included herein are methods, compositions, and systems for detecting infections (such as Aspergillus sp. infections and Mycobacterium sp. infections), or the risk thereof, as well as for treating such infections. Also provided are methods, compositions, and systems for detecting whether a subject who has pneumonia and is infected with HI has an increased risk of dying compared to a general population of subjects who have pneumonia and are infected with HIV. Methods, compositions, and systems for monitoring subjects diagnosted as having a disease or risk as disclosed herein are also included.

### CROSS-REFERENCE TO RELATED APPLICATION

This application claims the benefit of priority to U.S. Provisional Application No. 62/505,637, filed May 12, 2017, which is hereby incorporated by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant nos. All 14271, P01 AI089473 and U01 HL098964 awarded by the National Institutes of Health. The government has certain rights in the invention.

### INCORPORATION-BY-REFERENCE OF SEQUENCE LISTING

The content of the text file named “048536-600001 WO_SequenceListing.txt”, which was created on May 11, 2018, and is 852 bytes in size, is hereby incorporated by reference in its entirety.

### BACKGROUND

Even in the absence of acute respiratory infection, human immunodeficiency virus (HIV)-infected patients exhibit a broader breadth of lower airway bacterial taxa compared to that detected in healthy subjects (Iwai et al. J Clin Microbiol 2012; 50: 2995-3002; Twigg et al. American Journal of Respiratory and Critical Care Medicine 2016). Despite high morbidity and mortality within this population, little is known about whether variation in airway microbiota composition and immune response are related to patient outcome.

### BRIEF SUMMARY

Provided herein are, inter alia, methods, compositions, and systems, for detecting and treating lung dysbiosis. Included are methods, compositions, and systems for detecting an airway microbiome, the diversity of microorganisms, a plurality of microorganisms, and/or at least one metabolite in the a biological sample from a subject. In aspects, provided herein are methods, compositions, and systems for detecting infections (such as Aspergillus sp. infections and Mycobacterium sp. infections), or the risk thereof, as well as for treating and preventing such infections. Also provided are methods, compositions, and systems for detecting whether a subject who has pneumonia and is infected with HIV has an increased risk of dying compared to a general population of subjects who have pneumonia and are infected with HIV. Methods, compositions, and systems for monitoring subjects diagnosted as having a disease or risk as disclosed herein are also included.

In an aspect, a method of detecting dysbiosis in a lung of a subject is provided. In embodiments, the method includes detecting (i) a diversity of microorganisms in a biological sample from the subject; and/or (ii) a plurality of microorganisms in a biological sample from the subject.

In an aspect, a method of detecting an airway microbiome in a subject who has or is suspected of having a lung infection is provided. In embodiments, the method includes detecting bacteria, or a proportion of bacteria, in a biological sample from the subject that are in the family Prevotellaceae, Pseudomonadaceae, Sphingomonadaceae, Streptococcaceae, and/or Veillonellaceae.

In an aspect a method of detecting an airway microbiome in a subject who has or is suspected of having a lung infection is provided. In embodiments, the method includes detecting bacteria, or a proportion of bacteria, in a biological sample from the subject that are in the family Prevotellaceae, Pseudomonadaceae, Sphingomonadaceae, Streptococcaceae, and/or Veillonellaceae.

In an aspect, a method of detecting at least one immune protein or immune protein-encoding mRNA in a subject who has or is suspected of having a lung infection is provided. In embodiments, the method includes detecting the level of TIM-3 protein or TIM-3 mRNA in a biological sample from the subject.

In an aspect, method of detecting at least one immune protein or immune protein-encoding mRNA in a subject who is infected with HIV or is suspected of being infected with HIV is provided. In embodiments, the method includes detecting the level of TIM-3 protein or TIM-3 mRNA in a biological sample from the subject.

In an aspect, a method of detecting at least one metabolite in a subject who has or is suspected of having a lung infection is provided. In embodiments, the method includes detecting the at least one metabolite in a biological sample from the subject, wherein the at least one metabolite is a tryptophan metabolite, an arachidonic acid metabolite, an eicosanoid, or a product of primary or secondary bile metabolism.

In an aspect, a method of detecting at least one metabolite in a subject who is infected with HIV or is suspected of being infected with HIV is provided. In embodiments, the method includes detecting the at least one metabolite in a biological sample from the subject, wherein the at least one metabolite is a tryptophan metabolite, an arachidonic acid metabolite, an eicosanoid, or a product of primary or secondary bile metabolism.

In an aspect, a method of detecting whether a subject has an Aspergillus sp. infection is provided. In embodiments, the method includes detecting (i) a diversity of microorganisms in a biological sample from the subject; and/or (ii) a plurality of microorganisms in a biological sample from the subject.

In an aspect, a method of detecting whether a subject has a Mycobacterium sp. infection is provided. In embodiments, the method includes detecting (i) a diversity of microorganisms in a biological sample from the subject; and/or (ii) a plurality of microorganisms in a biological sample from the subject.

In an aspect, a method of detecting whether a subject is at risk of an Aspergillus sp. infection is provided. In embodiments, the method includes detecting (i) a diversity of microorganisms in a biological sample from the subject; and/or (ii) a plurality of microorganisms in a biological sample from the subject.

In an aspect, a method of detecting whether a subject is at risk of a Mycobacterium sp. infection is provided. In embodiments, the method includes detecting (i) a diversity of microorganisms in a biological sample from the subject; and/or (ii) a plurality of microorganisms in a biological sample from the subject.

In an aspect, a method of detecting whether a subject has an Aspergillus sp. infection is provided. In embodiments, the method includes detecting at least one metabolite in a biological sample from the subject.

In an aspect, a method of detecting whether a subject has a Mycobacterium sp. infection is provided. In embodiments, the method includes detecting at least one metabolite in a biological sample from the subject.

In an aspect, a method of detecting whether a subject is at risk of an Aspergillus sp. infection is provided. In embodiments, the method includes detecting at least one metabolite in a biological sample from the subject.

In an aspect, a method of detecting whether a subject is at risk of a Mycobacterium sp. infection is provided. In embodiments, the method includes detecting at least one metabolite in a biological sample from the subject.

In an aspect, included herein is a method of detecting whether a subject who has pneumonia and is infected with HIV has an increased risk of dying within about 1, 2, 3, 4, 5, 6, 7, 8, 9, or 10 weeks compared to a general population of subjects who have pneumonia and are infected with HIV. In embodiments, the method includes detecting (i) the diversity of microorganisms in a biological sample; (ii) a plurality of microorganisms in a biological sample; and/or (iii) at least one metabolite in a biological sample.

In an aspect, a method of detecting dysbiosis in the lungs is provided. In embodiments, the method includes (a) obtaining a biological sample from the subject; and (b) detecting (i) a diversity of microorganisms in the biological sample; and/or (ii) a plurality of microorganisms in the biological sample.

In an aspect, a method of detecting an airway microbiome in a subject who has or is suspected of having a lung infection is provided. In embodiments, the method includes (a) obtaining a biological sample from the subject; and (b) detecting bacteria, or a proportion of bacteria, in the biological sample that are in the family Prevotellaceae, Pseudomonadaceae, Sphingomonadaceae, Streptococcaceae, and/or Veillonellaceae.

In an aspect a method of detecting an airway microbiome in a subject who has or is suspected of having a lung infection is provided. In embodiments, the method includes (a) obtaining a biological sample from the subject; and (b) detecting bacteria, or a proportion of bacteria, in the biological sample that are in the family Prevotellaceae, Pseudomonadaceae, Sphingomonadaceae, Streptococcaceae, and/or Veillonellaceae.

In an aspect, a method of detecting at least one immune protein or immune protein-encoding mRNA in a subject who has or is suspected of having a lung infection is provided. In embodiments, the method includes: (a) obtaining a biological sample from the subject; and (b) detecting the level of TIM-3 protein or TIM-3 mRNA in the biological sample.

In an aspect, method of detecting at least one immune protein or immune protein-encoding mRNA in a subject who is infected with HIV or is suspected of being infected with HIV is provided. In embodiments, the method includes: (a) obtaining a biological sample from the subject; and (b) detecting the level of TIM-3 protein or TIM-3 mRNA in the biological sample.

In an aspect, a method of detecting at least one metabolite in a subject who has or is suspected of having a lung infection is provided. In embodiments, the method includes: (a) obtaining a biological sample from the subject; and (b) detecting the at least one metabolite in the biological sample, wherein the at least one metabolite is a tryptophan metabolite, an arachidonic acid metabolite, an eicosanoid, or a product of primary or secondary bile metabolism.

In an aspect, a method of detecting at least one metabolite in a subject who is infected with HIV or is suspected of being infected with HIV is provided. In embodiments, the method includes: (a) obtaining a biological sample from the subject; and (b) detecting the at least one metabolite in the biological sample, wherein the at least one metabolite is a tryptophan metabolite, an arachidonic acid metabolite, an eicosanoid, or a product of primary or secondary bile metabolism.

In an aspect, a method of detecting whether a subject has an Aspergillus sp. infection is provided. In embodiments, the method includes (a) obtaining a biological sample from the subject; and (b) detecting (i) a diversity of microorganisms in the biological sample; and/or (ii) a plurality of microorganisms in the biological sample.

In an aspect, a method of detecting whether a subject has a Mycobacterium sp. infection is provided. In embodiments, the method includes (a) obtaining a biological sample from the subject; and (b) detecting (i) a diversity of microorganisms in the biological sample; and/or (ii) a plurality of microorganisms in the biological sample.

In an aspect, a method of detecting whether a subject is at risk of an Aspergillus sp. infection is provided. In embodiments, the method includes (a) obtaining a biological sample from the subject; and (b) detecting (i) a diversity of microorganisms in the biological sample; and/or (ii) a plurality of microorganisms in the biological sample.

In an aspect, a method of detecting whether a subject is at risk of a Mycobacterium sp. infection is provided. In embodiments, the method includes (a) obtaining a biological sample from the subject; and (b) detecting (i) a diversity of microorganisms in the biological sample; and/or (ii) a plurality of microorganisms in the biological sample.

In an aspect, a method of detecting whether a subject has an Aspergillus sp. infection is provided. In embodiments, the method includes (a) obtaining a biological sample from the subject; and (b) detecting at least one metabolite in the biological sample.

In an aspect, a method of detecting whether a subject has a Mycobacterium sp. infection is provided. In embodiments, the method includes (a) obtaining a biological sample from the subject; and (b) detecting at least one metabolite in the biological sample.

In an aspect, a method of detecting whether a subject is at risk of an Aspergillus sp. infection is provided. In embodiments, the method includes (a) obtaining a biological sample from the subject; and (b) detecting at least one metabolite in the biological sample.

In an aspect, a method of detecting whether a subject is at risk of a Mycobacterium sp. infection is provided. In embodiments, the method includes (a) obtaining a biological sample from the subject; and (b) detecting at least one metabolite in the biological sample.

In an aspect, included herein is a method of detecting whether a subject who has pneumonia and is infected with HIV has an increased risk of dying within about 1, 2, 3, 4, 5, 6, 7, 8, 9, or 10 weeks compared to a general population of subjects who have pneumonia and are infected with HIV. In embodiments, the method includes (a) obtaining at least one biological sample from the subject; and (b) detecting (i) the diversity of microorganisms in a biological sample; (ii) a plurality of microorganisms in a biological sample; and/or (iii) at least one metabolite in a biological sample.

In an aspect, method of treating or preventing a lung infection in a subject in need thereof is provided. In embodiments, the method includes administering an effective amount of at least one antibiotic or antifungal agent to the subject. In embodiments, the subject (a) has an increased level of expression of 1 of or any combination of 2, 3, 4, 5, 6, 7, 8, 9, 10, 11, 12, 13, 14, 15, or 16 of IFNγ, IFNα, TNFα, MUC5AC, IL-17A, IL-4, IL-5, IL-13, IL-33, OCEL1, CCL11, FOXP3, PD-1, CD45RO, CD39, and/or GAPDH compared to a general or healthy population; (b) has an increased level of 1, 2, 3, or 5 or 3-methyl-2-oxobutyrate, 4-methyl-2-oxopentanoate, 1-dihomo-linolenylglycerol, 1-myristoylglycerol, or inosine compared to a general or healthy population of subjects; (c) has an increased proportion of lung microbiome bacteria in the Prevotellaceae family of bacteria compared to a healthy or general population of subjects; (d) has an increased proportion of lung microbiome bacteria in the Prevotellaceae family of bacteria compared to a healthy or general population of subjects, wherein the increased proportion is a proportion of at least about 10%, 20%, 30%, 40%, or 50%; (e) has a proportion of at least 30% of lung microbiome bacteria in the Prevotellaceae family of bacteria, at least 10% lung microbiome bacteria in the Streptococcaceae family of bacteria, and at least 10% lung microbiome bacteria in the Veillonellaceae family of bacteria; (f) has increased TIM-3 expression compared to a general or healthy population of subjects; (g) has an increased level of 1, 2, 3, 4, 5, 6, or 7 of leukotriene B4, xanthurenate, 15-hydroxyeicosatetraenoic acid, chenodeoxycholate, glycodeoxycholate, taurochenodeoxycholate, and/or ursodeoxycholate compared to a general or healthy population of subjects; (h) has an increased proportion of lung microbiome bacteria in the Pseudomonadaceae family of bacteria compared to a healthy or general population of subjects; (i) has an increased proportion of lung microbiome bacteria in the Pseudomonadaceae family of bacteria compared to a healthy or general population of subjects, wherein the increased proportion is a proportion of at least about 10%, 20%, 30%, 40%, or 50%; and/or (j) has a proportion of at least 40% of lung microbiome bacteria in the Pseudomonadaceae family of bacteria, at least 10% of lung microbiome bacteria in the Sphingomonadaceae family of bacteria, and at least 5% of lung microbiome bacteria in the Prevotellaceae family of bacteria.

In an aspect, a method of treating or preventing a lung infection in a subject in need thereof us provided. In embodiments, the method includes (a) detecting (i) an airway microbiome; (ii) the diversity of microorganisms; (iii) a plurality of microorganisms; and/or (iv) at least one metabolite, in at least one biological sample from the subject; and (b) administering to the subject an effective amount of at least one antibiotic or antifungal agent.

In an aspect, a method of treating or preventing a Mycobacterium sp. infection in a subject in need thereof is provided. In embodiments, the method includes administering an effective amount of at least one antibiotic compound to the subject. In embodiments, the subject (a) has increased T-cell immunoglobulin and mucin-domain containing-3 (TIM-3) expression compared to a general or healthy population of subjects; (b) has an increased level of 1, 2, 3, 4, 5, 6, or 7 of leukotriene B4, xanthurenate, 15-hydroxyeicosatetraenoic acid, chenodeoxycholate, glycodeoxycholate, taurochenodeoxycholate, and/or ursodeoxycholate compared to a general or healthy population of subjects; (c) has an increased proportion of lung microbiome bacteria in the Pseudomonadaceae family of bacteria compared to a healthy or general population of subjects; (d) has an increased proportion of lung microbiome bacteria in the Pseudomonadaceae family of bacteria compared to a healthy or general population of subjects, wherein the increased proportion is a proportion of at least about 10%, 20%, 30%, 40%, or 50%; and/or (e) has a proportion of at least 40% of lung microbiome bacteria in the Pseudomonadaceae family of bacteria, at least 10% of lung microbiome bacteria in the Sphingomonadaceae family of bacteria, and at least 5% of lung microbiome bacteria in the Prevotellaceae family of bacteria. The “proportion” of a bacterial type (e.g., family, genus, species, or other taxon) in a population of bacterial cells is the percentage of the total number of bacterial cells that are of the bacterial type.

In an aspect, a method of treating or preventing a Mycobacterium sp. infection in a subject in need thereof is provided. In embodiments, the method includes (a) detecting (i) the diversity of microorganisms; (ii) a plurality of microorganisms; and/or (iii) at least one metabolite, in at least one biological sample from the subject; and (b) administering to the subject an effective amount of at least one antibiotic compound.

In an aspect, a method of treating or preventing an Aspergillus sp. infection in a subject in need thereof is provided. In embodiments, the method includes administering an effective amount of at least one antifungal agent to the subject. In embodiments, the subject (a) has an increased level of expression of 1 of or any combination of 2, 3, 4, 5, 6, 7, 8, 9, 10, 11, 12, 13, 14, 15, or 16 of IFNγ, IFNα, TNFα, MUC5AC, IL-17A, IL-4, IL-5, IL-13, IL-33, OCEL1, CCL11, FOXP3, PD-1, CD45RO, CD39, and/or GAPDH compared to a general or healthy population; (b) has an increased level of 1, 2, 3, or 5 or 3-methyl-2-oxobutyrate, 4-methyl-2-oxopentanoate, 1-dihomo-linolenylglycerol, 1-myristoylglycerol, or inosine compared to a general or healthy population of subjects; (c) has an increased proportion of lung microbiome bacteria in the Prevotellaceae family of bacteria compared to a healthy or general population of subjects; (d) has an increased proportion of lung microbiome bacteria in the Prevotellaceae family of bacteria compared to a healthy or general population of subjects, wherein the increased proportion is a proportion of at least about 10%, 20%, 30%, 40%, or 50%; and/or (e) has a proportion of at least 30% of lung microbiome bacteria in the Prevotellaceae family of bacteria, at least 10% lung microbiome bacteria in the Streptococcaceae family of bacteria, and at least 10% lung microbiome bacteria in the Veillonellaceae family of bacteria.

In an aspect, a method of treating or preventing an Aspergillus sp. infection in a subject in need thereof is provided. In embodiments, the method includes (a) detecting (i) the diversity of microorganisms; (ii) a plurality of microorganisms; and/or (iii) at least one metabolite, in at least one biological sample from the subject; and (b) administering to the subject an effective amount of at least one antifungal agent.

### BRIEF DESCRIPTION OF THE DRAWINGS

FIGS. 1A-1C. Antibiotic administration, alpha-diversity, and probabilistic modeling differentiate bacterial community types within the lower airways of HIV-pneumonia patients. PERMANOVA analyses of n=182 lower airway BAL bacterial community profiles of Ugandan HIV-pneumonia patients indicate that (FIG. 1A) Ceftriaxone (shown in light gray versus no ceftriaxone in dark gray), a third generation cephalosporin, administered at time of bronchoscopy is significantly associated with community composition (PERMANOVA, R²=0.061, p<0.001). Shannon diversity (PERMANOVA, R²=0.17, p<0.001. FIG. 1B: Based on Laplace approximation, where a lower value indicates a better model fit, Dirichlet Multinomial Mixtures identified two compositionally distinct bacterial microbiota (n=136 and n=46) in the lower airways of HIV-pneumonia patients. FIG. 1C: Principal coordinates analysis illustrates that DMM-defined lower airway bacterial communities are compositionally distinct (PERMANOVA, R²=0.246, p<0.001).

FIG. 2. Two compositionally distinct lower airway microbial states exist in HIV-infected pneumonia patients. Lower airway phylogenetic diversity differs significantly across microbial states (one-way ANOVA, p<0.001).

FIGS. 3A-3D. Culture positivity for Mycobacterium or Aspergillus, as well as antibiotic administration and mortality differ between MCS. Mycobacterium (Chi-squared, p=0.006; FIG. 3A) or (FIG. 3B) Aspergillus (p=0.07) culture positivity, (FIG. 3C) ceftriaxone administration at bronchoscopy (p<0.0001), and (FIG. 3D) mortality after 1 week of enrollment (p=0.08) differ amongst microbial states (positive in black, negative in gray).

FIGS. 4A-4H. Airway microbial states are predicted to encode distinct metagenomes, and each is shown to induce different lower airway immunological responses and is associated with significantly different serum metabolomes. FIGS. 4A-4H: Comparative LC/MS metabolomic analysis of paired patient serum identified 60 metabolites that differed significantly between all three groups (Kruskal-Wallis; p<0.05). FIG. 4A: Carbohydrates. FIG. 4B: Energy.

FIG. 4C: Cofactors and vitamins. FIG. 4D: Xenobiotics. FIG. 4E: Nucleotides. FIG. 4F: Peptides. FIG. 4G: Amino Acids. FIG. 4H: Lipids.

FIG. 5. Sequence read depth and observed species distributions. Rarefaction curves showing total observed species per sample across multiple read depths, indicates that 100,000 reads results in good community coverage, demonstrated by curves approaching a plateau.

FIGS. 6A-D. Clinical and demographic factors are significantly associated with community composition. Principal coordinates analyses (PCoAs) comparing community composition of the lower airways to (FIG. 6A) gender (PERMANOVA, R²=0.021, p<0.017), (FIG. 6B) alcohol ever consumed (PERMANOVA, R²=0.015, p<0.045), (FIG. 6C) Aspergillus positive culture (PERMANOVA, R²=0.038, p<0.004), and (FIG. 6D) Mycobacterium positive culture (PERMANOVA, R²=0.027, p<0.021).

FIG. 7. Microbial community states are associated with mortality outcomes. KaplanMeier curves comparing mortality among microbial states from enrollment for 70 days (p>0.05).

### DETAILED DESCRIPTION

### I. Definitions

In While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Subject” “Patient” or “subject in need thereof” refers to a living member of the animal kingdom suffering from or that may suffer from the indicated disorder. In embodiments, the subject is a member of a species that includes individuals who naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human. In embodiments, the subject is a non-mammalian animal such as a turkey, a duck, or a chicken. In embodiments, a subject is a living organism suffering from or prone to a disease or condition that can be treated by administration of a composition or pharmaceutical composition as provided herein.

As used herein, a “symptom” of a disease includes any clinical or laboratory manifestation associated with the disease, and is not limited to what a subject can feel or observe.

In the context of a lung, the term “dysbiosis” means a difference in the lung microbiota compared to a healthy or general population. In embodiments, dysbiosis includes a difference in lung microbiota commensal species diversity compared to a healthy or general population. In embodiments, dysbiosis includes a decrease of beneficial microorganisms and/or increase of pathobionts (pathogenic or potentially pathogenic microorganisms) and/or decrease of overall microbiota species diversity. Many factors can harm the beneficial members of the lung microbiota leading to dysbiosis, including (but not limited to) infection, antibiotic use, psychological and physical stress, radiation, and dietary changes. In embodiments, dysbiosis includes or promotes the overgrowth of a pathogen such as M. tuberculosis, A. fumigatus, or A. flavus. In embodiments, the dysbiosis includes an increased amount (absolute number or proportion of the total microbial population) of bacterial or fungal cells of a species or genus (e.g., 5%, 10%, 15%, 20%, 25%, 30%, 35%, 40%, 45%, 50%, 55%, 60%, 65%, 70%, 75%, 80%, 85%, 90%, 95% or more lower) compared to a healthy subject (e.g., a corresponding subject who does not have HIV or pneumonia, and who has not been administered an antibiotic within about 1, 2, 3, 4, 5, or 6 months, and/or compared to a healthy or general population). In embodiments, the dysbiosis includes an increased amount (absolute number or proportion of the total microbial population) of bacterial or fungal cells within a species or genus (e.g., 5%, 10%, 15%, 20%, 25%, 30%, 35%, 40%, 45%, 50%, 55%, 60%, 65%, 70%, 75%, 80%, 85%, 90%, 95% or more higher) compared to a healthy subject (e.g., a corresponding subject who does not have HIV or pneumonia, and who has not been administered an antibiotic within about 1, 2, 3, 4, 5, or 6 months, and/or compared to a healthy or general population). In embodiments, a subject who has HIV or pneumonia or who has received an antibiotic within about 1, 2, 3, 4, 5, 6, 7, 8, 9, or 10 weeks is deemed to have lung dysbiosis. In embodiments, antibiotic administration (e.g., systemically, such as by intravenous injection or orally) is causing or has caused a major alteration in the normal microbiota. Thus, as used herein, the term “antibiotic-induced dysbiosis” refers to dysbiosis (e.g., in the lung) caused by or following the administration of an antibiotic.

Non-limiting examples of dysbiosis are described in the examples provided herein. Non-limiting examples of dysbiosis in the context of subjects with HIV and pneumonia are also described in Shenoy et al. (2017) “Immune Response and Mortality Risk Relate to Distinct Lung Microbiomes in HIV-Pneumonia Patients” Am J Respir Crit Care Med 195(1): 104-114, PMID: 27447987, PMCID: PMC5214918, originally published in press as DOI: 10.1164/rccm.201603-0523OC on Jul. 22, 2016 (hereinafter “Shenoy et al. 2017”), the entire content of which (including all supplemental information and data) is incorporated herein by reference. In some embodiments, a subject with dysbiosis has the MCS1, MCS2A, or MCS2B microbiome profile as set forth in Shenoy et al. 2017.

A “control” or “standard control” refers to a sample, measurement, or value that serves as a reference, usually a known reference, for comparison to a test sample, measurement, or value. For example, a test sample can be taken from a patient suspected of having a given disease (e.g. dysbiosis, HIV, pneumonia, HIV and pneumonia, or other disease) and compared to a known normal (non-diseased) individual (e.g. a standard control subject). A standard control can also represent an average measurement or value gathered from a population of similar individuals (e.g. standard control subjects) that do not have a given disease (e.g. standard control population), e.g., healthy individuals with a similar medical background, same age, weight, etc. In embodiments, a standard control represents an average measurement or value gathered from a general population of similar individuals (e.g. standard control subjects) that have a given disease (e.g. standard control population), e.g., with a similar medical background, same age, weight, etc. (such as individuals with HIV, pneumonia, or both HIV and pneumonia). In embodiments, a standard control is a proportion, level, or amount (e.g., an average proportion, level, or amount) in a healthy or general population of subjects. In embodiments, a general population of subjects is a general population of subjects in a geographical area (such as a country or continent, e.g., Asia, Australia, Africa, North America, South America, or Europe). In embodiments, a general population of subjects is a general population of subjects in (e.g., that self-identify as being within) an ethnic group such as caucasian (e.g., white), African, of African descent (e.g., African American), Native American, Asian, or of Asian descent. In embodiments, a general population of subjects is a general population of subjects without a disease such as an HIV infection. In embodiments, a general population of subjects is a general population of subjects with a disease such as an HIV infection. In embodiments, a general population of subjects is a general population of subjects with an HIV infection and pneumonia. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. microbiome, RNA levels, protein levels, specific cell types, specific bodily fluids, specific tissues, metabolites, etc.).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

The term “diagnosis” refers to a determination or relative probability that a disease (e.g. dysbiosis, an infection, or other disease) is present in the subject. In embodiments, a subject is diagnosed with a disease when the disease has been detected (e.g., with an assay) in a subject. Similarly, the term “prognosis” refers to a relative probability that a certain future outcome may occur in the subject with respect to a disease state. For example, in the context of the present disclosure, prognosis can refer to the likelihood that an individual will develop a disease (e.g. dysbiosis, infection, or other disease), or the likely severity of the disease (e.g., duration of disease or mortality within a given timeframe). The terms are not intended to be absolute, as will be appreciated by any one of skill in the field of medical diagnostics.

“Biological sample” or “sample” refers to materials obtained from or derived from a subject or patient. In embodiments, a biological sample is or includes a bronchoalveolar lavage (BAL) sample, sputum, phlegm, saliva, or mucus. In embodiments, a biological sample is or includes a bodily fluid. In embodiments, a biological sample is or includes blood, serum, or plasma. In embodiments, a biological samples is or includes blood, a blood fraction, or product (e.g., serum, plasma, platelets, red blood cells, and the like). In embodiments, a biological sample is or includes tissue, such as lung tissue. In embodiments, a sample is obtained from a eukaryotic organism, such as a mammal such as a primate e.g., chimpanzee or human; cow; dog; cat; a rodent, e.g., guinea pig, rat, or mouse; rabbit; or a bird; reptile; or fish. In embodiments, a biological sample includes sections of tissues such as biopsy and autopsy samples, and frozen sections taken for histological purposes.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

As used herein the abbreviation “sp.” for species means at least one species (e.g., 1, 2, 3, 4, 5, or more species) of the indicated genus. The abbreviation “spp.” for species means 2 or more species (e.g., 2, 3, 4, 5, 6, 7, 8, 9, 10 or more) of the indicated genus. In embodiments, methods and compositions provided herein include a single species within an indicated genus or indicated genera, or 2 or more (e.g., a plurality including more than 2) species within an indicated genus or indicated genera. In embodiments, 1, 2, 3, 4, 5, or more or all or the indicated species is or are isolated. In embodiments, the indicated species are administered together. In embodiments, each of the indicated species is present in a single composition that includes each of the species. In embodiments, each of the species is administered concurrently, e.g., within about 1, 2, 3, 4, 5, 6, 7, 8, 9, 10, 30, or 60, 1-5, 1-10, 1-30, 1-60, or 5-15 seconds or minutes of each other.

The phrase “stringent hybridization conditions” refers to conditions under which a primer or probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium).

Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

In embodiments, nucleic acids that do not hybridize to each other under stringent conditions are still considdered substantially identical if the polypeptides which they encode are substantially identical. This occurs, for example, when a copy of a nucleic acid is created using the maximum codon degeneracy permitted by the genetic code. In embodiments, the nucleic acids hybridize under moderately stringent hybridization conditions. Exemplary “moderately stringent hybridization conditions” include a hybridization in a buffer of 40% formamide, 1 M NaCl, 1% SDS at 370° C., and a wash in 1×SSC at 450° C. A positive hybridization is at least twice background. Those of ordinary skill will readily recognize that alternative hybridization and wash conditions can be utilized to provide conditions of similar stringency. Additional guidelines for determining hybridization parameters are provided in numerous references, e.g., Current Protocols in Molecular Biology, ed. Ausubel, et al., supra.

In embodiments, detecting includes an assay. In embodiments, the assay is an analytic procedure to qualitatively assess or quantitatively measure the presence, amount, or functional activity of an entity, element, or feature (e.g., a compound, a level of gene expression, a bacterial type or taxon, or a bacterial population such as in a microbiome). In embodiments, assaying the level of a compound (such as a protein, an mRNA molecule, or a metabolite) includes using an analytic procedure (such as an in vitro procedure) to qualitatively assess or quantitatively measure the presence or amount of the compound.

In this disclosure, “comprises,” “comprising,” “containing,” and “having” and the like can have the meaning ascribed to them in U.S. Patent law and can mean “includes,” “including,” and the like. Thus, the transitional term “comprising,” which is synonymous with “including,” “containing,” or “characterized by,” is inclusive or open-ended and does not exclude additional, unrecited features, integers, steps, operations, elements, and/or components. “Consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. By contrast, the transitional phrase “consisting of” excludes any feature, integer, element, step, operation, component, and/or ingredient not specified.

As used herein, the term “about” in the context of a numerical value or range means+10% of the numerical value or range recited or claimed, unless the context requires a more limited range.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

As used in the description herein and throughout the claims that follow, the meaning of “a,” “an,” and “the” includes plural reference unless the context clearly dictates otherwise.

### II. Methods of Detecting Dysbiosis, an Airway Microbiome, an Immune Protein, a Metabolite, and Risk of Infection

In an aspect, a method of detecting dysbiosis in a lung of a subject is provided. In embodiments, the method includes detecting (i) a diversity of microorganisms in a biological sample from the subject; and/or (ii) a plurality of microorganisms in a biological sample from the subject.

In an aspect, a method of detecting an airway microbiome in a subject who has or is suspected of having a lung infection is provided. In embodiments, the method includes detecting bacteria, or a proportion of bacteria, in a biological sample from the subject that are in the family Prevotellaceae, Pseudomonadaceae, Sphingomonadaceae, Streptococcaceae, and/or Veillonellaceae. In embodiments, the method further includes detecting the diversity of microorganisms in the biological sample.

In an aspect a method of detecting an airway microbiome in a subject who has or is suspected of having a lung infection is provided. In embodiments, the method includes detecting bacteria, or a proportion of bacteria, in a biological sample from the subject that are in the family Prevotellaceae, Pseudomonadaceae, Sphingomonadaceae, Streptococcaceae, and/or Veillonellaceae. In embodiments, the method further includes detecting the diversity of microorganisms in the biological sample.

In an aspect, a method of detecting at least one immune protein or immune protein-encoding mRNA in a subject who has or is suspected of having a lung infection is provided. In embodiments, the method includes detecting the level of TIM-3 protein or TIM-3 mRNA in a biological sample from the subject.

In an aspect, method of detecting at least one immune protein or immune protein-encoding mRNA in a subject who is infected with HIV or is suspected of being infected with HIV is provided. In embodiments, the method include detecting the level of TIM-3 protein or TIM-3 mRNA in a biological sample from the subject.

In an aspect, provided herein is a method of detecting whether a subject has or is at risk of an Aspergillus sp. or a Mycobacterium sp. infection. In embodiments, the method includes detecting (i) a diversity of microorganisms in a biological sample from the subject; and/or (ii) a plurality of microorganisms in a biological sample from the subject.

In an aspect, a method of detecting whether a subject has an Aspergillus sp. infection is provided. In embodiments, the method includes detecting (i) a diversity of microorganisms in a biological sample from the subject; and/or (ii) a plurality of microorganisms in a biological sample from the subject.

In an aspect, a method of detecting whether a subject has a Mycobacterium sp. infection is provided. In embodiments, the method includes detecting (i) a diversity of microorganisms in a biological sample from the subject; and/or (ii) a plurality of microorganisms in a biological sample from the subject.

In an aspect, a method of detecting whether a subject is at risk of an Aspergillus sp. infection is provided. In embodiments, the method includes detecting (i) a diversity of microorganisms in a biological sample from the subject; and/or (ii) a plurality of microorganisms in a biological sample from the subject.

In an aspect, a method of detecting whether a subject is at risk of a Mycobacterium sp. infection is provided. In embodiments, the method includes detecting (i) a diversity of microorganisms in a biological sample from the subject; and/or (ii) a plurality of microorganisms in a biological sample from the subject.

In embodiments, the method includes obtaining the biological sample from the subject. In embodiments, obtaining the biological sample from the subject comprises collecting the biological sample directly from the subject. In embodiments, obtaining the biological sample from the subject comprises receiving a biological sample that has been collected (e.g., directly) from the subject (e.g., by another actor, such as a clinical professional such as a nurse, medic, or doctor). In embodiments, the biological sample has been submitted by the subject (e.g., by mail or currior).

In embodiments, distinct pathogenic microbiota exist in patients with infection, e.g., acute infection (such as subjects who have HIV). In embodiments, distinct microbiota states induce distinct but reproducible immune dysfunction and are associated with significant differences in clinical responses. In embodiments, this approach is used to stratify patients in cohorts of patients with distinct infectious diseases, risks, diagnoses, and/or prognoses.

In embodiments, methods provided herein provide for the detection or understanding of patient heterogeneity. In embodiments, methods included herein tailor therapy to the specific microbiota dysbiosis and immune dysfunction presented by the patient.

In an aspect, a precision medicine application is provided. In embodiments, patient samples are tested to identify the specific pathogenic microbiota and therapy is tailored based on test results.

Current approaches to stratify patients fail to consider the pathogenic microbiota that is responsible for the observed immune dysfunction. Approaches provided herein permit identification of patient microbial endotypes whose treatment either stand alone or as an adjuvant to existing therapy will improve patient outcomes. In embodiments, methods provided herein provide relatively inexpensive technology, relatively rapid turn-around, opportunity for precision medicine.

In embodiments, the subject is infected with HIV. In embodiments, the subject has pneumonia. In embodiments, the pneumonia is bacterial pneumonia. In embodiments, the subject has tuberculosis (TB). In embodiments, the subject has TB pneumonia.

In embodiments, the subject has been administered an antibiotic. In embodiments, the antibiotic is ceftriaxone.

In embodiments, the biological sample is a bronchoalveolar lavage (BAL) sample, sputum, phlegm, saliva, or mucus.

In embodiments, the microorganisms are bacterial microorganisms.

In embodiments, detecting the diversity of microorganisms in the biological sample includes characterizing the microbiome in the biological sample.

In embodiments, a shotgun metagenomics and/or transcriptomic approach is used to identify, characterize, detect, or determine a metagenome. In embodiments, a shotgun metagenomics and/or transcriptomic approach is used to identify, characterize, detect, or determine viral and fungal taxa in a subject. In embodiments, metagenomics, in parallel with metabolomics and transcriptomics is used to stratify subjects based on their microbiomes.

In embodiments, by examining microbial a diverse immune profiles that exists within a patient population is identified, characterized, detected, or determined. In embodiments, the microbial and immunological features described herein inform strategies for tailored therapy in a patient population.

In embodiments, by examining microbial population (e.g., characterizing a microbiome), a diverse immune profile that exists within a patient population is identified, characterized, detected, or determined. In embodiments, the microbial and immunological features described herein inform strategies for tailored therapy in a patient population.

In embodiments, detecting the airway microbiome in the biological sample includes amplifying and sequencing 16S rRNA genes of microorganisms in the sample. In embodiments, detecting the diversity of microorganisms in the biological sample or a plurality of microorganisms in the biological sample includes amplifying and sequencing 16S rRNA genes of microorganisms in the sample.

In embodiments, detecting the airway microbiome in the biological sample inclues amplifying and sequencing the V4 region of 16S rRNA genes of microorganisms in the sample. In embodiments, detecting the diversity of microorganisms in the biological sample or a plurality of microorganisms in the biological sample includes amplifying and sequencing the V4 region of 16S rRNA genes of microorganisms in the sample.

In embodiments, the airway microbiome is a lung microbiome. In embodiments, detecting a plurality of microorganisms in the biological sample comprises characterizing a microbiome in the biological sample.

In embodiments, characterizing the microbiome in the biological sample comprises determining the number and/or identity of bacterial taxa represented by bacteria in the biological sample. In embodiments, the bacterial taxa comprise bacterial families, genera, and/or species. In embodiments, the bacterial taxa comprise, consist essentially of, or consist of bacterial families. In embodiments, the bacterial taxa are bacterial families. In embodiments, the bacterial taxa comprise, consist essentially of, or consist of bacterial genera. In embodiments, the bacterial taxa are bacterial genera. In embodiments, the bacterial taxa comprise, consist essentially of, or consist of bacterial species. In embodiments, the bacterial taxa are bacterial species. In embodiments, the bacterial taxa are bacterial families and/or genera.

In embodiments, the microorganisms are bacterial microorganisms, and detecting the diversity of microorganisms in the biological sample or a plurality of microorganisms in the biological sample includes detecting bacteria, or a proportion of bacteria, that are in the family Prevotellaceae, Pseudomonadaceae, Sphingomonadaceae, Streptococcaceae, and/or Veillonellaceae.

In embodiments, the method further includes determining the expression level of at least one immune gene (e.g., 1, 2, 3, 4, 5, 6, 7, 8, 9, 10, 11, 12, 13, 14, 15, 16, 17, 18, 19, or 20 immune genes) in the subject.

In embodiments, the at least one immune gene is 1 or or any combination of 2, 3, 4, 5, 6, 7, 8, 9, 10, 11, 12, 13, 14, 15, 16, 17, 18, 19, or 20 of interferon gamma (IFNγ), interferon alpha (IFNα), tumor necrosis factor alpha (TNFα), Mucin 5AC (MUCSAC), Interleukin 17A (IL-17A), Interleukin 4 (IL-4), Interleukin 5 (IL-5), Interleukin 13 (IL-13), Interleukin 33 (IL-33), Occludin/ELL Domain-Containing Protein 1 (OCEL1), C—C motif chemokine 11 (CCL11), Peptidyl Arginine Deiminase 4 (PADI4), Interleukin 10 (IL-10), Forkhead Box P3 (FOXP3), Programmed cell death protein 1 (PD-1), T-cell immunoglobulin and mucin-domain containing-3 (TIM-3), Protein Tyrosine Phosphatase, Receptor Type C isoform CD45RO (CD45RO), Cluster of Differentiation 2 (CD2), Cluster of Differentiation 39 (CD39), and Glyceraldehyde 3-phosphate dehydrogenase (GAPDH). In embodiments, the at least one immune gene is IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, or GAPDH. In embodiments, the at least one immune gene is any combination of 2 of IFNγ, IFNα, TNFα, MUCSAC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 3 of IFNγ, IFNα, TNFα, MUCSAC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 4 of IFNγ, IFNα, TNFα, MUCSAC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 5 of IFNγ, IFNα, TNFα, MUCSAC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 6 of IFNγ, IFNα, TNFα, MUCSAC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 7 of IFNγ, IFNα, TNFα, MUCSAC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 8 of IFNγ, IFNα, TNFα, MUCSAC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 9 of IFNγ, IFNα, TNFα, MUCSAC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 10 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 11 of IFNγ, IFNα, TNFα, MUC5AC, IL-17A, IL-4, IL-5, IL-13, IL-33, OCEL1, CCL1, PADI4, IL-10, FOXP3, PD-1, TIM-3, CD45RO, CD2, CD39, and GAPDH. In embodiments, the at least one immune gene is any combination of 12 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 13 of IFNγ, IFNα, TNFα, MUC5AC, IL-17A, IL-4, IL-5, IL-13, IL-33, OCEL1, CCL1, PADI4, IL-10, FOXP3, PD-1, TIM-3, CD45RO, CD2, CD39, and GAPDH. In embodiments, the at least one immune gene is any combination of 14 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 15 of IFNγ, IFNα, TNFα, MUC5AC, IL-17A, IL-4, IL-5, IL-13, IL-33, OCEL1, CCL1, PADI4, IL-10, FOXP3, PD-1, TIM-3, CD45RO, CD2, CD39, and GAPDH. In embodiments, the at least one immune gene is any combination of 16 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 17 of IFNγ, IFNα, TNFα, MUC5AC, IL-17A, IL-4, IL-5, IL-13, IL-33, OCEL1, CCL1, PADI4, IL-10, FOXP3, PD-1, TIM-3, CD45RO, CD2, CD39, and GAPDH. In embodiments, the at least one immune gene is any combination of 18 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 19 of IFNγ, IFNα, TNFα, MUC5AC, IL-17A, IL-4, IL-5, IL-13, IL-33, OCEL1, CCL1, PADI4, IL-10, FOXP3, PD-1, TIM-3, CD45RO, CD2, CD39, and GAPDH. In embodiments, the at least one immune gene is each of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH.

In embodiments, the method includes determining whether the level TIM-3 protein or TIM-3 mRNA is increased compared to a general or healthy population of subjects.

In embodiments, the method further includes detecting the level of any one of or any combination of 2, 3, 4, 5, 6, 7, 8, 9, 10, 11, 12, 13, 14, 15, 16, 17, 18, or 19 of (i) IFNγ protein, IFN protein, TNF protein, MUC5AC protein, IL-17A protein, IL-4 protein, IL-5 protein, IL-13 protein, IL-33 protein, OCEL1 protein, CCL11 protein, PADI4 protein, IL-10 protein, FOXP3 protein, PD-1 protein, CD45RO protein, CD2 protein, CD39 protein, and GAPDH protein; or (ii) IFNγ mRNA, IFNα mRNA, TNFα mRNA, MUCSAC mRNA, IL-17A mRNA, IL-4 mRNA, IL-5 mRNA, IL-13 mRNA, IL-33 mRNA, OCEL1 mRNA, CCL11 mRNA, PADI4 mRNA, IL-10 mRNA, FOXP3 mRNA, PD-1 mRNA, CD45RO mRNA, CD2 mRNA, CD39 mRNA, and GAPDH mRNA, in the biological sample. In embodiments, no more than 1000, 900, 800, 700, 600, 500, 400, 300, 200, 100, 50, 20, or 10 proteins or mRNAs are detected. In embodiments, the detecting does not comprise the use of a microarray. In embodiments, the detecting comprises the use of a microarray that detects the mRNA levels of less than 1000, 900, 800, 700, 600, 500, 400, 300, 200, 100, 50, 20, or 10 genes.

In embodiments, detecting the diversity of microorganisms in the biological sample includes determining the number of families, the number of genera, the number of species, the Faith's Phylogenetic Diversity, the Shannon Diversity, and/or the Simpson Diversity of the microorganisms.

In embodiments, detecting the airway microbiome in the biological sample includes detecting whether the biological sample has an increased proportion of bacteria in the Pseudomonadaceae family of bacteria compared to a general population of subjects who have pneumonia and are infected with HIV. In embodiments, detecting the airway microbiome in the biological sample includes detecting whether the biological sample has an increased proportion of bacteria in the Pseudomonadaceae family of bacteria compared to a general population of subjects who have pneumonia and are infected with HIV, wherein the increased proportion is a proportion of at least about 10%, 20%, 30%, 40%, or 50%. In embodiments, detecting the airway microbiome in the biological sample includes detecting whether at least 40% of the bacteria in the biological sample are in the Pseudomonadaceae family of bacteria, at least 10% of the bacteria in the biological sample are in the Sphingomonadaceae family of bacteria, and at least 5% of the bacteria in the biological sample are in the Prevotellaceae family of bacteria. In embodiments, detecting the airway microbiome in the biological sample includes detecting whether the biological sample has an increased proportion of bacteria in the Streptococcaceae family of bacteria compared to a general population of subjects who have pneumonia and are infected with HIV. In embodiments, detecting the airway microbiome in the biological sample includes detecting whether the biological sample has an increased proportion of bacteria in the Streptococcaceae family of bacteria compared to a general population of subjects who have pneumonia and are infected with HIV, wherein the increased proportion is a proportion of at least about 10%, 20%, 30%, 40%, or 50%. In embodiments, detecting the airway microbiome in the biological sample includes detecting whether at least 40% of the bacteria in the biological sample are in the Streptococcaceae family of bacteria, at least 5% of the bacteria in the biological sample are in the Veillonellaceae family of bacteria, and at least 10% of the bacteria in the biological sample are in the Prevotellaceae family of bacteria. In embodiments, detecting the airway microbiome in the biological sample includes detecting whether the biological sample has an increased proportion of bacteria in the Prevotellaceae family of bacteria compared to a general population of subjects who have pneumonia and are infected with HIV. In embodiments, detecting the airway microbiome in the biological sample includes detecting whether the biological sample has an increased proportion of bacteria in the Prevotellaceae family of bacteria compared to a general population of subjects who have pneumonia and are infected with HIV, wherein the increased proportion is a proportion of at least about 10%, 20%, 30%, 40%, or 50%. In embodiments, detecting the airway microbiome in the biological sample includes detecting whether at least 30% of the bacteria in the biological sample are in the Prevotellaceae family of bacteria, at least 10% of the bacteria in the biological sample are in the Streptococcaceae family of bacteria, and at least 10% of the bacteria in the biological sample are in the Veillonellaceae family of bacteria.

In embodiments, detecting the airway microbiome in the biological sample includes detecting whether the subject has an increased proportion of lung microbiome bacteria in the Pseudomonadaceae family of bacteria compared to a healthy or general population of subjects. In embodiments, detecting the airway microbiome in the biological sample includes detecting whether the subject has an increased proportion of lung microbiome bacteria in the Pseudomonadaceae family of bacteria compared to a healthy or general population of subjects, wherein the increased proportion is a proportion of at least about 10%, 20%, 30%, 40%, or 50%. In embodiments, detecting the airway microbiome in the biological sample includes detecting whether the subject has a proportion of at least 40% of lung microbiome bacteria in the Pseudomonadaceae family of bacteria, at least 10% of lung microbiome bacteria in the Sphingomonadaceae family of bacteria, and at least 5% of lung microbiome bacteria in the Prevotellaceae family of bacteria.

In embodiments, detecting the airway microbiome in the biological sample includes detecting whether the subject has an increased proportion of lung microbiome bacteria in the Pseudomonadaceae family of bacteria compared to a healthy or general population of subjects. In embodiments, detecting the airway microbiome in the biological sample includes detecting whether the subject has an increased proportion of lung microbiome bacteria in the Pseudomonadaceae family of bacteria compared to a healthy or general population of subjects, wherein the increased proportion is a proportion of at least about 10%, 20%, 30%, 40%, or 50%. In embodiments, detecting the airway microbiome in the biological sample includes detecting whether the subject has a proportion of at least 40% of lung microbiome bacteria in the Pseudomonadaceae family of bacteria, at least 10% of lung microbiome bacteria in the Sphingomonadaceae family of bacteria, and at least 5% of lung microbiome bacteria in the Prevotellaceae family of bacteria.

In embodiments, detecting the airway microbiome in the biological sample includes detecting whether the subject has an increased proportion of lung microbiome bacteria in the Prevotellaceae family of bacteria compared to a healthy or general population of subjects. In embodiments, detecting the airway microbiome in the biological sample includes detecting whether the subject has an increased proportion of lung microbiome bacteria in the Prevotellaceae family of bacteria compared to a healthy or general population of subjects, wherein the increased proportion is a proportion of at least about 10%, 20%, 30%, 40%, or 50%. In embodiments, detecting the airway microbiome in the biological sample includes detecting whether the subject has a proportion of at least 30% of lung microbiome bacteria in the Prevotellaceae family of bacteria, at least 10% lung microbiome bacteria in the Streptococcaceae family of bacteria, and at least 10% lung microbiome bacteria in the Veillonellaceae family of bacteria.

In embodiments, detecting the airway microbiome in the biological sample includes detecting whether the subject has an increased proportion of lung microbiome bacteria in the Prevotellaceae family of bacteria compared to the general population. In embodiments, detecting the airway microbiome in the biological sample includes detecting whether the subject has an increased proportion of lung microbiome bacteria in the Prevotellaceae family of bacteria compared to the general population, wherein the increased proportion is a proportion of at least about 10%, 20%, 30%, 40%, or 50%. In embodiments, detecting the airway microbiome in the biological sample includes detecting whether the subject has a proportion of at least 30% of lung microbiome bacteria in the Prevotellaceae family of bacteria, at least 10% of lung microbiome bacteria in the Streptococcaceae family of bacteria, and at least 10% of lung microbiome bacteria in the Veillonellaceae family of bacteria.

In embodiments, detecting the diversity of microorganisms in the biological sample or a plurality of microorganisms in the biological sample includes detecting whether the biological sample has an increased proportion of bacteria in the Pseudomonadaceae family of bacteria compared to a standard control (such as the proportion in a healthy or general population of subjects). In embodiments, detecting the diversity of microorganisms in the biological sample or a plurality of microorganisms in the biological sample includes detecting whether the biological sample has an increased proportion of bacteria in the Pseudomonadaceae family of bacteria compared to a standard control (such as the proportion in a healthy or general population of subjects). In embodiments, the increased proportion is a proportion of at least about 10%, 20%, 30%, 40%, or 50%. In embodiments, detecting the diversity of microorganisms in the biological sample or a plurality of microorganisms in the biological sample includes detecting whether at least 40% of the bacteria in the biological sample are in the Pseudomonadaceae family of bacteria, at least 10% of the bacteria in the biological sample are in the Sphingomonadaceae family of bacteria, and at least 5% of the bacteria in the biological sample are in the Prevotellaceae family of bacteria. In embodiments, detecting the diversity of microorganisms in the biological sample or a plurality of microorganisms in the biological sample includes detecting whether the biological sample has an increased proportion of bacteria in the Streptococcaceae family of bacteria compared to a standard control (such as the proportion in a healthy or general population of subjects). In embodiments, detecting the diversity of microorganisms in the biological sample or a plurality of microorganisms in the biological sample includes detecting whether the biological sample has an increased proportion of bacteria in the Streptococcaceae family of bacteria compared to a standard control (such as the proportion in a healthy or general population of subjects). In embodiments, the increased proportion is a proportion of at least about 10%, 20%, 30%, 40%, or 50%. In embodiments, detecting the diversity of microorganisms in the biological sample or a plurality of microorganisms in the biological sample includes detecting whether at least 40% of the bacteria in the biological sample are in the Streptococcaceae family of bacteria, at least 5% of the bacteria in the biological sample are in the Veillonellaceae family of bacteria, and at least 10% of the bacteria in the biological sample are in the Prevotellaceae family of bacteria. In embodiments, detecting the diversity of microorganisms in the biological sample or a plurality of microorganisms in the biological sample includes detecting whether the biological sample has an increased proportion of bacteria in the Prevotellaceae family of bacteria compared to a standard control (such as the proportion in a healthy or general population of subjects). In embodiments, detecting the diversity of microorganisms in the biological sample or a plurality of microorganisms in the biological sample includes detecting whether the biological sample has an increased proportion of bacteria in the Prevotellaceae family of bacteria compared to a standard control (such as the proportion in a healthy or general population of subjects). In embodiments, the increased proportion is a proportion of at least about 10%, 20%, 30%, 40%, or 50%. In embodiments, detecting the diversity of microorganisms in the biological sample or a plurality of microorganisms in the biological sample includes detecting whether at least 30% of the bacteria in the biological sample are in the Prevotellaceae family of bacteria, at least 10% of the bacteria in the biological sample are in the Streptococcaceae family of bacteria, and at least 10% of the bacteria in the biological sample are in the Veillonellaceae family of bacteria.

In embodiments, the subject has pneumonia and is infected with HIV, and detecting whether the subject is at risk of an Aspergillus sp. or a Mycobacterium sp. infection includes detecting an increased risk of an Aspergillus sp. or Mycobacterium sp. infection compared to a standard control. In embodiments, the standard control is the rate or likelihood of infection in a general population of subjects who have pneumonia and are infected with HIV. In embodiments, the subject has pneumonia and is infected with HIV, and detecting whether the subject is at risk of an Aspergillus sp. or a Mycobacterium sp. infection includes detecting an increased risk of an Aspergillus sp. or Mycobacterium sp. infection compared to a general population of subjects who have pneumonia and are infected with HIV.

In embodiments, detecting the diversity of microorganisms in the biological sample or a plurality of microorganisms in the biological sample includes detecting whether the biological sample has an increased proportion of bacteria in the Pseudomonadaceae family of bacteria compared to a standard control (such as the proportion in a general population of subjects who have pneumonia and are infected with HIV). In embodiments, detecting the diversity of microorganisms in the biological sample or a plurality of microorganisms in the biological sample includes detecting whether the biological sample has an increased proportion of bacteria in the Pseudomonadaceae family of bacteria compared to a standard control (such as the proportion in a general population of subjects who have pneumonia and are infected with HIV). In embodiments, the increased proportion is a proportion of at least about 10%, 20%, 30%, 40%, or 50%. In embodiments, detecting the diversity of microorganisms in the biological sample or a plurality of microorganisms in the biological sample includes detecting whether at least 40% of the bacteria in the biological sample are in the Pseudomonadaceae family of bacteria, at least 10% of the bacteria in the biological sample are in the Sphingomonadaceae family of bacteria, and at least 5% of the bacteria in the biological sample are in the Prevotellaceae family of bacteria. In embodiments, detecting the diversity of microorganisms in the biological sample or a plurality of microorganisms in the biological sample includes detecting whether the biological sample has an increased proportion of bacteria in the Streptococcaceae family of bacteria compared to a standard control (such as the proportion in general population of subjects who have pneumonia and are infected with HIV). In embodiments, detecting the diversity of microorganisms in the biological sample or a plurality of microorganisms in the biological sample includes detecting whether the biological sample has an increased proportion of bacteria in the Streptococcaceae family of bacteria compared to a standard control (such as the proportion in a general population of subjects who have pneumonia and are infected with HIV). In embodiments, the increased proportion is a proportion of at least about 10%, 20%, 30%, 40%, or 50%. In embodiments, detecting the diversity of microorganisms in the biological sample or a plurality of microorganisms in the biological sample includes detecting whether at least 40% of the bacteria in the biological sample are in the Streptococcaceae family of bacteria, at least 5% of the bacteria in the biological sample are in the Veillonellaceae family of bacteria, and at least 10% of the bacteria in the biological sample are in the Prevotellaceae family of bacteria. In embodiments, detecting the diversity of microorganisms in the biological sample or a plurality of microorganisms in the biological sample includes detecting whether the biological sample has an increased proportion of bacteria in the Prevotellaceae family of bacteria compared to a standard control (such as the proportion in a general population of subjects who have pneumonia and are infected with HIV). In embodiments, detecting the diversity of microorganisms in the biological sample or a plurality of microorganisms in the biological sample includes detecting whether the biological sample has an increased proportion of bacteria in the Prevotellaceae family of bacteria compared to a standard control (such as the proportion in a general population of subjects who have pneumonia and are infected with HIV). In embodiments, the increased proportion is a proportion of at least about 10%, 20%, 30%, 40%, or 50%. In embodiments, detecting the diversity of microorganisms in the biological sample or a plurality of microorganisms in the biological sample includes detecting whether at least 30% of the bacteria in the biological sample are in the Prevotellaceae family of bacteria, at least 10% of the bacteria in the biological sample are in the Streptococcaceae family of bacteria, and at least 10% of the bacteria in the biological sample are in the Veillonellaceae family of bacteria. In embodiments, detecting the diversity of microorganisms in the biological sample or a plurality of microorganisms in the biological sample includes detecting whether (a) the biological sample has an increased proportion of bacteria in the Pseudomonadaceae family of bacteria compared to a general population of subjects who have pneumonia and are infected with HIV; (b) the biological sample has an increased proportion of bacteria in the Pseudomonadaceae family of bacteria compared to a general population of subjects who have pneumonia and are infected with HIV, wherein the increased proportion is a proportion of at least about 10%, 20%, 30%, 40%, or 50%; (c) at least 40% of the bacteria in the biological sample are in the Pseudomonadaceae family of bacteria, at least 10% of the bacteria in the biological sample are in the Sphingomonadaceae family of bacteria, and at least 5% of the bacteria in the biological sample are in the Prevotellaceae family of bacteria; (d) the biological sample has an increased proportion of bacteria in the Streptococcaceae family of bacteria compared to a general population of subjects who have pneumonia and are infected with HIV; (e) the biological sample has an increased proportion of bacteria in the Streptococcaceae family of bacteria compared to a general population of subjects who have pneumonia and are infected with HIV, wherein the increased proportion is a proportion of at least about 10%, 20%, 30%, 40%, or 50%; (f) at least 40% of the bacteria in the biological sample are in the Streptococcaceae family of bacteria, at least 5% of the bacteria in the biological sample are in the Veillonellaceae family of bacteria, and at least 10% of the bacteria in the biological sample are in the Prevotellaceae family of bacteria; (g) the biological sample has an increased proportion of bacteria in the Prevotellaceae family of bacteria compared to a general population of subjects who have pneumonia and are infected with HIV; (h) the biological sample has an increased proportion of bacteria in the Prevotellaceae family of bacteria compared to a general population of subjects who have pneumonia and are infected with HIV, wherein the increased proportion is a proportion of at least about 10%, 20%, 30%, 40%, or 50%; and/or (i) at least 30% of the bacteria in the biological sample are in the Prevotellaceae family of bacteria, at least 10% of the bacteria in the biological sample are in the Streptococcaceae family of bacteria, and at least 10% of the bacteria in the biological sample are in the Veillonellaceae family of bacteria.

In embodiments, the biological sample is from an airway of the subject. In embodiments, the airway is in a lung of the subject. In embodiments, the airway is a trachea, bronchus, bronchiole, alveolar duct, alveolar sac, and/or alveolus. In embodiments, the biological sample is a bronchoalveolar lavage (BAL) sample, sputum, phlegm, saliva, or mucus.

In an aspect, a method of detecting at least one metabolite in a subject who has or is suspected of having a lung infection is provided. In embodiments, the method includes: detecting the at least one metabolite in a biological sample from the subject, wherein the at least one metabolite is a tryptophan metabolite, an arachidonic acid metabolite, an eicosanoid, or a product of primary or secondary bile metabolism.

In an aspect, a method of detecting at least one metabolite in a subject who is infected with HIV or is suspected of being infected with HIV is provided. In embodiments, the method includes detecting the at least one metabolite in a biological sample from the subject, wherein the at least one metabolite is a tryptophan metabolite, an arachidonic acid metabolite, an eicosanoid, or a product of primary or secondary bile metabolism.

In embodiments, the biological sample is a bodily fluid. In embodiments, the bodily fluid is blood, serum, or plasma. In embodiments, the at least one metabolite is leukotriene B4, xanthurenate, 15-hydroxyeicosatetraenoic acid, chenodeoxycholate, glycodeoxycholate, taurochenodeoxycholate, or ursodeoxycholate. In embodiments, the at least one metabolite is a lysolipid metabolite, a pyrimidine metabolite, or a monoacylglycerol. In embodiments, the at least one metabolite is a valine metabolite, a leucine metabolite, or a monoacylglycerol associated with lipid metabolism. In embodiments, the at least one metabolite is 3-methyl-2-oxobutyrate, 4-methyl-2-oxopentanoate, 1-dihomo-linolenylglycerol, 1-myristoylglycerol, or inosine. In embodiments, the method includes detecting whether the subject has an increased level of 1, 2, 3, 4, 5, 6, or 7 of leukotriene B4, xanthurenate, 15-hydroxyeicosatetraenoic acid, chenodeoxycholate, glycodeoxycholate, taurochenodeoxycholate, and/or ursodeoxycholate compared to a general or healthy population of subjects. In embodiments, the method includes detecting whether the subject has an increased level of 1, 2, 3, 4, 5, 6, or 7 of leukotriene B4, xanthurenate, 15-hydroxyeicosatetraenoic acid, chenodeoxycholate, glycodeoxycholate, taurochenodeoxycholate, and/or ursodeoxycholate compared to a general or healthy population of subjects. In embodiments, the method includes detecting whether the subject has an increased level of 1, 2, 3, or 5 or 3-methyl-2-oxobutyrate, 4-methyl-2-oxopentanoate, 1-dihomo-linolenylglycerol, 1-myristoylglycerol, or inosine compared to a general or healthy population of subjects. In embodiments, the method includes detecting whether the subject has an increased level of 1, 2, 3, or 5 or 3-methyl-2-oxobutyrate, 4-methyl-2-oxopentanoate, 1-dihomo-linolenylglycerol, 1-myristoylglycerol, or inosine compared to the general population.

In embodiments, the method further includes detecting at least one metabolite in the biological sample.

In embodiments, the method further includes detecting at least one metabolite in an additional biological sample from the subject. In embodiments, the method further includes obtaining an additional biological sample from the subject, and detecting at least one metabolite in the additional biological sample. In embodiments, the additional biological sample is a bodily fluid. In embodiments, the bodily fluid is blood, serum, or plasma.

In an aspect, a method of detecting whether a subject has or is at risk of an Aspergillus sp. or a Mycobacterium sp. infection is provided. In embodiments, the method includes detecting at least one metabolite in a biological sample from the subject.

In an aspect, a method of detecting whether a subject has an Aspergillus sp. infection is provided. In embodiments, the method includes detecting at least one metabolite in a biological sample from the subject.

In an aspect, a method of detecting whether a subject has a Mycobacterium sp. infection is provided. In embodiments, the method includes detecting at least one metabolite in a biological sample from the subject.

In an aspect, a method of detecting whether a subject is at risk of an Aspergillus sp. infection is provided. In embodiments, the method includes detecting at least one metabolite in a biological sample from the subject.

In an aspect, a method of detecting whether a subject is at risk of a Mycobacterium sp. infection is provided. In embodiments, the method includes detecting at least one metabolite in a biological sample from the subject.

In embodiments, the method includes obtaining the biological sample from the subject. In embodiments, obtaining the biological sample from the subject comprises collecting the biological sample directly from the subject. In embodiments, obtaining the biological sample from the subject comprises receiving a biological sample that has been collected (e.g., directly) from the subject (e.g., by another actor, such as a clinical professional such as a nurse, medic, or doctor). In embodiments, the biological sample has been submitted by the subject (e.g., by mail or currior).

In embodiments, the method includes obtaining the biological sample from the subject. In embodiments, obtaining the biological sample from the subject comprises collecting the biological sample directly from the subject. In embodiments, obtaining the biological sample from the subject comprises receiving a biological sample that has been collected (e.g, directly) from the subject (e.g., by another actor, such as a clinical professional such as a nurse, medic, or doctor). In embodiments, the biological sample has been submitted by the subject (e.g., by mail or currior).

In embodiments, the subject is infected with HIV or is suspected of being infected with HIV. In embodiments, the subject has or is suspected of having pneumonia. In embodiments, the subject has bacterial pneumonia or fungal pneumonia. In embodiments, the subject has or is suspected of having TB. In embodiments, the subject has or is suspected of having TB pneumonia. In embodiments, the subject has been administered an antibiotic. In embodiments, the antibiotic is ceftriaxone.

In embodiments, the biological sample is a bodily fluid.

In embodiments, the bodily fluid is blood, serum, or plasma.

In embodiments, the metabolite is a plurality of metabolites.

In embodiments, the metabolite is any 1 of or any combination of (e.g., any combination of 2, 3, 4, 5, 6, 7, 8, 9, 10, 15, 20, 30 or more of): 4-guanidinobutanoate, cis-urocanate, trans-urocanate, xanthurenate, glucose, 1-methylnicotinamide, biopterin, 15-HETE,leukotriene B4, eicosanodioate, maleate (cis-Butenedioate), 13-HODE+9-HODE, scyllo-inositol, 1-palmitoylglycerophosphate, 1-palmitoylglycerol (1-monopalmitin), 1-stearoylglycerol (1-monostearin), 2-palmitoylglycerol (2-monopalmitin), glycerophosphoinositol, chenodeoxycholate, glycochenodeoxycholate, taurochenodeoxycholate, glycocholenate sulfate, glycodeoxycholate, glycolithocholate sulfate, glycoursodeoxycholate, taurocholenate sulfate, taurodeoxycholate, ursodeoxycholate, 21-hydroxypregnenolone disulfate, 5alpha-pregnan-3(alpha or beta), 20beta-diol disulfate, pregnen-diol disulfate, N6-methyladenosine, 4-ureidobutyrate, glycylphenylalanine, glycylvaline, lysyltyrosine, phenylalanylaspartate, tyrosyllysine, thymol sulfate, 1-methylxanthine, 7-methylxanthine, Theobromine, N-acetylphenylalanine, Maltose, 1-margaroylglycerophosphoethanolamine, 1-palmitoylglycerophosphoglycerol, 2-stearoylglycerophosphoethanolamine, 5alpha-pregnan-3beta,20alpha-diol disulfate, N4-acetylcytidine, 3-methyl-2-oxobutyrate, 4-methyl-2-oxopentanoate, methionine sulfone, 1-dihomo-linolenylglycerol (alpha, gamma), 1-myristoylglycerol (1-monomyristin), inosine, phenylalanyltryptophan, phenylalanine, 1-palmitoylglycerophosphoethanolamine, glycolithocholate, and alpha-ketoglutarate.

In embodiments, the metabolite is a tryptophan metabolite, an arachidonic acid metabolite, an eicosanoid, or a product of primary or secondary bile metabolism.

In embodiments, the metabolite is leukotriene B4, xanthurenate, 15-hydroxyeicosatetraenoic acid, chenodeoxycholate, glycodeoxycholate, taurochenodeoxycholate, or ursodeoxycholate.

In embodiments, the metabolite is a lysolipid metabolite, a pyrimidine metabolite, or a monoacylglycerol.

In embodiments, the metabolite is a valine metabolite, a leucine metabolite, or a monoacylglycerol associated with lipid metabolism.

In embodiments, the metabolite is 3-methyl-2-oxobutyrate, 4-methyl-2-oxopentanoate, 1-dihomo-linolenylglycerol, 1-myristoylglycerol, or inosine.

In embodiments, the method further includes detecting at least one metabolite in an additional biological sample from the subject. In embodiments, the method further includes obtaining an additional biological sample from the subject, and detecting (i) the diversity of microorganisms in the additional biological sample and/or (ii) a plurality of microorganisms in the biological sample in the additional biological sample.

In embodiments, the additional biological sample is a bronchoalveolar lavage (BAL) sample, sputum, phlegm, saliva, or mucus.

In embodiments, the method further includes identifying the subject as having dysbiosis if the subject has an increased proportion of lung microbiome bacteria in the Pseudomonadaceae family of bacteria compared to a standard control (such as a healthy or general population of subjects). In embodiments, the method further includes identifying the subject as having dysbiosis if the subject has an increased proportion of lung microbiome bacteria in the Pseudomonadaceae family of bacteria compared to a standard control (such as a healthy or general population of subjects). In embodiments, the increased proportion is a proportion of at least about 10%, 20%, 30%, 40%, or 50%. In embodiments, the method further includes identifying the subject as having dysbiosis if the subject has a proportion of at least 40% of lung microbiome bacteria in the Pseudomonadaceae family of bacteria, at least 10% of lung microbiome bacteria in the Sphingomonadaceae family of bacteria, and at least 5% of lung microbiome bacteria in the Prevotellaceae family of bacteria.

In embodiments, the method further includes identifying the subject as having or at risk of a Mycobacterium sp. infection if the subject has increased TIM-3 expression compared to a standard control (such as the level of expression in general or healthy population of subjects). In embodiments, the method further includes identifying the subject as having or at risk of a Mycobacterium sp. infection if the subject has an increased level of 1 or or any combination of 2, 3, 4, 5, 6, or 7 of leukotriene B4, xanthurenate, 15-hydroxyeicosatetraenoic acid, chenodeoxycholate, glycodeoxycholate, taurochenodeoxycholate, and/or ursodeoxycholate compared to a standard control (such as the level in a general or healthy population of subjects). In embodiments, the method further includes identifying the subject as having or at risk of a Mycobacterium sp. infection if the subject has an increased proportion of lung microbiome bacteria in the Pseudomonadaceae family of bacteria compared to a standard control (such as the proportion in a healthy or general population of subjects). In embodiments, the method further includes identifying the subject as having or at risk of a Mycobacterium sp. infection if the subject has an increased proportion of lung microbiome bacteria in the Pseudomonadaceae family of bacteria compared to a standard control (such as a healthy or general population of subjects). In embodiments, the increased proportion is a proportion of at least about 10%, 20%, 30%, 40%, or 50%. In embodiments, the method further includes identifying the subject as having or at risk of a Mycobacterium sp. infection if the subject has a proportion of at least 40% of lung microbiome bacteria in the Pseudomonadaceae family of bacteria, at least 10% of lung microbiome bacteria in the Sphingomonadaceae family of bacteria, and at least 5% of lung microbiome bacteria in the Prevotellaceae family of bacteria. In embodiments, the method further includes identifying the subject as having or at risk of a Mycobacterium sp. infection if the subject: (a) has increased TIM-3 expression compared to a general or healthy population of subjects; (b) has an increased level of 1, 2, 3, 4, 5, 6, or 7 of leukotriene B4, xanthurenate, 15-hydroxyeicosatetraenoic acid, chenodeoxycholate, glycodeoxycholate, taurochenodeoxycholate, and/or ursodeoxycholate compared to a general or healthy population of subjects; (c) has an increased proportion of lung microbiome bacteria in the Pseudomonadaceae family of bacteria compared to a healthy or general population of subjects; (d) has an increased proportion of lung microbiome bacteria in the Pseudomonadaceae family of bacteria compared to a healthy or general population of subjects, wherein the increased proportion is a proportion of at least about 10%, 20%, 30%, 40%, or 50%; and/or (e) has a proportion of at least 40% of lung microbiome bacteria in the Pseudomonadaceae family of bacteria, at least 10% of lung microbiome bacteria in the Sphingomonadaceae family of bacteria, and at least 5% of lung microbiome bacteria in the Prevotellaceae family of bacteria. In embodiments, the general population is a general population of subjects who have pneumonia and are infected with HIV.

In embodiments, the method further includes identifying the subject as at risk of an Aspergillus sp. infection if the subject has an increased level of expression of 1 of or any combination of 2, 3, 4, 5, 6, 7, 8, 9, 10, 11, 12, 13, 14, 15, or 16 of IFNγ, IFNα, TNFα, MUC5AC, IL-17A, IL-4, IL-5, IL-13, IL-33, OCEL1, CCL11, FOXP3, PD-1, CD45RO, CD39, and/or GAPDH compared to a standard control (such as the level in a general or healthy population). In embodiments, the method further includes identifying the subject as at risk of an Aspergillus sp. infection if the subject has an increased level of 1, 2, 3, or 5 or 3-methyl-2-oxobutyrate, 4-methyl-2-oxopentanoate, 1-dihomo-linolenylglycerol, 1-myristoylglycerol, or inosine compared to a standard control (such as a general or healthy population of subjects). In embodiments, the method further includes identifying the subject as at risk of an Aspergillus sp. infection if the subject has an increased proportion of lung microbiome bacteria in the Prevotellaceae family of bacteria compared to a standard control (such as the proportion in a healthy or general population of subjects). In embodiments, the method further includes identifying the subject as at risk of an Aspergillus sp. infection if the subject has an increased proportion of lung microbiome bacteria in the Prevotellaceae family of bacteria compared to a standard control (such as a healthy or general population of subjects). In embodiments, the increased proportion is a proportion of at least about 10%, 20%, 30%, 40%, or 50%. In embodiments, the method further includes identifying the subject as at risk of an Aspergillus sp. infection if the subject has a proportion of at least 30% of lung microbiome bacteria in the Prevotellaceae family of bacteria, at least 10% lung microbiome bacteria in the Streptococcaceae family of bacteria, and at least 10% lung microbiome bacteria in the Veillonellaceae family of bacteria. In embodiments, the method further includes identifying the subject as at risk of an Aspergillus sp. infection if the subject: (a) has an increased level of expression of 1 of or any combination of 2, 3, 4, 5, 6, 7, 8, 9, 10, 11, 12, 13, 14, 15, or 16 of IFNγ, IFNα, TNFα, MUC5AC, IL-17A, IL-4, IL-5, IL-13, IL-33, OCEL1, CCL11, FOXP3, PD-1, CD45RO, CD39, and/or GAPDH compared to a general or healthy population; (b) has an increased level of 1, 2, 3, or 5 or 3-methyl-2-oxobutyrate, 4-methyl-2-oxopentanoate, 1-dihomo-linolenylglycerol, 1-myristoylglycerol, or inosine compared to a general or healthy population of subjects; (c) has an increased proportion of lung microbiome bacteria in the Prevotellaceae family of bacteria compared to a healthy or general population of subjects; (d) has an increased proportion of lung microbiome bacteria in the Prevotellaceae family of bacteria compared to a healthy or general population of subjects, wherein the increased proportion is a proportion of at least about 10%, 20%, 30%, 40%, or 50%; and/or (e) has a proportion of at least 30% of lung microbiome bacteria in the Prevotellaceae family of bacteria, at least 10% lung microbiome bacteria in the Streptococcaceae family of bacteria, and at least 10% lung microbiome bacteria in the Veillonellaceae family of bacteria. In embodiments, the general population is a general population of subjects who have pneumonia and are infected with HIV.

In embodiments, the Mycobacterium sp. is M. tuberculosis.

In embodiments, the Aspergillus sp. is A. fumigatus or A. flavus.

In embodiments, a biological sample is a bodily fluid obtained by filtration and/or centrifugation. For example, the biological sample may be a filtrate of e.g., blood, a BAL sample, sputum, phlegm, saliva, or mucus, or the supernatant of a centrifuged BAL sample or centrifuged blood, sputum, phlegm, saliva, or mucus. In embodiments, a filtrate is centrifuged. In embodiments a supernatant is filtered. In embodiments, centrifugation is used to increase the passage of a fluid through a filter. Non-limiting examples of filters include filters that restrict any molecule greater than, e.g., 50, 100, 200, 300, 400, 500, 50-500, 50-100, 100-500 nm in diameter (or average diameter), or greater than 0.5, 1, 1.5, 2, 2.5, 5, 10, 15, 25, 50, 100, or 200 microns in diameter (e.g., average diameter). In embodiments, a filter has pores of about 50, 100, 200, 300, 400, 500, 50-500, 50-100, 100-500 nm in diameter or about 0.5, 1, 1.5, 2, 2.5, 5, 10, 15, 25, 50, 100, or 200 microns in diameter.

In embodiments, detecting a compound (e.g., a metabolite or a protein) and/or the expression level thereof includes Ultrahigh Performance Liquid Chromatography-Tandem Mass Spectrometry (UPLC-MS/MS), Gas Chromatography-Mass Spectrometry (GC-MS), high performance liquid chromatography (HPLC), gas chromatography, liquid chromatography, Mass spectrometry (MS), inductively coupled plasma-mass spectrometry (ICP-MS), accelerator mass spectrometry (AMS), thermal ionization-mass spectrometry (TIMS) and spark source mass spectrometry (SSMS), matrix-assisted laser desorption/ionization (MALDI), and/or MALDI-TOF.

In embodiments, detecting the expression level of a protein includes assaying the level of the compound (e.g., with high-performance liquid chromatography (HPLC), liquid chromatography-mass spectrometry (LC/MS), an enzyme-linked immunosorbent assay (ELISA), protein immunoprecipitation, immunoelectrophoresis, protein immunostaining, and/or Western blot) or the level of mRNA that encodes the protein. In embodiments, detecting the expression level of a compound (e.g., a protein) includes lysing a cell. In embodiments, detecting the expression level of a compound includes a polymerase chain reaction (e.g., reverse transcriptase polymerase chain reaction), RNA sequencing, microarray analysis, immunohistochemistry, or flow cytometry.

In an aspect, provided herein is a kit or system for performing a diagnostic method disclosed herein. In embodiments, the kit or system includes one or more primers, probes, or antibodies specific for a protein or any combination of any one of the proteins mentioned herein. In embodiments, the kit or system includes one or more primers or probes specific for the mRNA of a protein or any combination of any one of the proteins mentioned herein. In embodiments, the kit or system includes one or more primers or probes specific for one or more of any combination of the bacterial species, genera, families or other taxa, disclosed herein. In embodiments, the one or more probes or primers hybridize the 16S rRNA gene of one or more bacterial taxa disclosed herein under stringent hybridization conditions.

### III. Methods of Detecting Increased Risk of Death

In an aspect, provided herein is a method of detecting whether a subject who has pneumonia and is infected with HIV has an increased risk of dying within about 1, 2, 3, 4, 5, 6, 7, 8, 9, or 10 weeks compared to a general population of subjects who have pneumonia and are infected with HIV. In embodiments, a subject who has pneumonia and is infected with HIV is identified as having an increased risk of dying within about 1 week compared to a general population of subjects who have pneumonia and are infected with HIV. In embodiments, a subject who has pneumonia and is infected with HIV is identified as having an increased risk of dying within about 2 weeks compared to a general population of subjects who have pneumonia and are infected with HIV. In embodiments, a subject who has pneumonia and is infected with HIV is identified as having an increased risk of dying within about 3 weeks compared to a general population of subjects who have pneumonia and are infected with HIV. In embodiments, a subject who has pneumonia and is infected with HIV is identified as having an increased risk of dying within about 4 weeks compared to a general population of subjects who have pneumonia and are infected with HIV. In embodiments, a subject who has pneumonia and is infected with HIV is identified as having an increased risk of dying within about 5 weeks compared to a general population of subjects who have pneumonia and are infected with HIV. In embodiments, a subject who has pneumonia and is infected with HIV is identified as having an increased risk of dying within about 6 weeks compared to a general population of subjects who have pneumonia and are infected with HIV. In embodiments, a subject who has pneumonia and is infected with HIV is identified as having an increased risk of dying within about 7 weeks compared to a general population of subjects who have pneumonia and are infected with HIV. In embodiments, a subject who has pneumonia and is infected with HIV is identified as having an increased risk of dying within about 8 weeks compared to a general population of subjects who have pneumonia and are infected with HIV. In embodiments, a subject who has pneumonia and is infected with HIV is identified as having an increased risk of dying within about 9 weeks compared to a general population of subjects who have pneumonia and are infected with HIV. In embodiments, a subject who has pneumonia and is infected with HIV is identified as having an increased risk of dying within about 10 weeks compared to a general population of subjects who have pneumonia and are infected with HIV.

In embodiments, the method includes detecting (i) the diversity of microorganisms in a biological sample; (ii) a plurality of microorganisms in a biological sample; and/or (iii) at least one metabolite in a biological sample. In embodiments, the method includes detecting the diversity of microorganisms in the biological sample. In embodiments, the method includes detecting a plurality of microorganisms in the biological sample. In embodiments, the method includes detecting at least one metabolite in the biological sample. In embodiments, the method includes detecting (i) the diversity of microorganisms in the biological sample; and/or (ii) a plurality of microorganisms in the biological sample. In embodiments, the method includes detecting at least one metabolite in an additional biological sample from the subject. In embodiments, the method includes obtaining one or more biological samples from the subject. In embodiments, obtaining a biological sample from the subject comprises collecting the biological sample directly from the subject. In embodiments, obtaining a biological sample from the subject comprises receiving a biological sample that has been collected (e.g, directly) from the subject (e.g., by another actor, such as a clinical professional such as a nurse, medic, or doctor). In embodiments, the biological sample has been submitted by the subject (e.g., by mail or currior).

In embodiments, the pneumonia is bacterial pneumonia. In embodiments, the subject has tuberculosis (TB). In embodiments, the subject has TB pneumonia.

In embodiments, the subject has been administered an antibiotic. In embodiments, the antibiotic is ceftriaxone.

In embodiments, the biological sample is a bronchoalveolar lavage (BAL) sample, sputum, phlegm, saliva, or mucus.

In embodiments, the microorganisms are bacterial microorganisms.

In embodiments, detecting the diversity of microorganisms in the biological sample includes characterizing the microbiome in the biological sample.

In embodiments, detecting the diversity of microorganisms in the biological sample or a plurality of microorganisms in the biological sample includes amplifying and sequencing 16S rRNA genes of microorganisms in the sample.

In embodiments, detecting the diversity of microorganisms in the biological sample or a plurality of microorganisms in the biological sample includes amplifying and sequencing the V4 region of 16S rRNA genes of microorganisms in the sample.

In embodiments, the microorganisms are bacterial microorganisms, and detecting the diversity of microorganisms in the biological sample or a plurality of microorganisms in the biological sample includes detecting bacteria, or a proportion of bacteria, that are in the family Prevotellaceae, Pseudomonadaceae, Sphingomonadaceae, Streptococcaceae, and/or Veillonellaceae.

In embodiments, the method further includes determining the expression level of at least one immune gene (e.g., 1, 2, 3, 4, 5, 6, 7, 8, 9, 10, 11, 12, 13, 14, 15, 16, 17, 18, 19, or 20 immune genes) in the subject.

In embodiments, the at least one immune gene is any 1 or or any combination of 2, 3, 4, 5, 6, 7, 8, 9, 10, 11, 12, 13, 14, 15, 16, 17, 18, 19, or 20 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, or GAPDH. In embodiments, the at least one immune gene is any combination of 2 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 3 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 4 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 5 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 6 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 7 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 8 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 9 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 10 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 11 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 12 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 13 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 14 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 15 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 16 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 17 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 18 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is any combination of 19 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH. In embodiments, the at least one immune gene is each of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH.

In embodiments, detecting the diversity of microorganisms in the biological sample includes determining the number of families, the number of genera, the number of species, the Faith's Phylogenetic Diversity, the Shannon Diversity, and/or the Simpson Diversity of the microorganisms.

In embodiments, detecting the diversity of microorganisms in the biological sample or a plurality of microorganisms in the biological sample includes detecting whether the biological sample has an increased proportion of bacteria in the Pseudomonadaceae family of bacteria compared to a standard control (such as the proportion in a general population of subjects who have pneumonia and are infected with HIV). In embodiments, detecting the diversity of microorganisms in the biological sample or a plurality of microorganisms in the biological sample includes detecting whether the biological sample has an increased proportion of bacteria in the Pseudomonadaceae family of bacteria compared to a standard control (such as the proportion in a general population of subjects who have pneumonia and are infected with HIV). In embodiments, the increased proportion is a proportion of at least about 10%, 20%, 30%, 40%, or 50%. In embodiments, detecting the diversity of microorganisms in the biological sample or a plurality of microorganisms in the biological sample includes detecting whether at least 40% of the bacteria in the biological sample are in the Pseudomonadaceae family of bacteria, at least 10% of the bacteria in the biological sample are in the Sphingomonadaceae family of bacteria, and at least 5% of the bacteria in the biological sample are in the Prevotellaceae family of bacteria. In embodiments, detecting the diversity of microorganisms in the biological sample or a plurality of microorganisms in the biological sample includes detecting whether the biological sample has an increased proportion of bacteria in the Streptococcaceae family of bacteria compared to a standard control (such as the proportion in a general population of subjects who have pneumonia and are infected with HIV). In embodiments, detecting the diversity of microorganisms in the biological sample or a plurality of microorganisms in the biological sample includes detecting whether the biological sample has an increased proportion of bacteria in the Streptococcaceae family of bacteria compared to a standard control (such as the proportion in a general population of subjects who have pneumonia and are infected with HIV). In embodiments, the increased proportion is a proportion of at least about 10%, 20%, 30%, 40%, or 50%. In embodiments, detecting the diversity of microorganisms in the biological sample or a plurality of microorganisms in the biological sample includes detecting whether at least 40% of the bacteria in the biological sample are in the Streptococcaceae family of bacteria, at least 5% of the bacteria in the biological sample are in the Veillonellaceae family of bacteria, and at least 10% of the bacteria in the biological sample are in the Prevotellaceae family of bacteria. In embodiments, detecting the diversity of microorganisms in the biological sample or a plurality of microorganisms in the biological sample includes detecting whether the biological sample has an increased proportion of bacteria in the Prevotellaceae family of bacteria compared to a standard control (such as the proportion in a general population of subjects who have pneumonia and are infected with HIV). In embodiments, detecting the diversity of microorganisms in the biological sample or a plurality of microorganisms in the biological sample includes detecting whether the biological sample has an increased proportion of bacteria in the Prevotellaceae family of bacteria compared to a standard control (such as the proportion in a general population of subjects who have pneumonia and are infected with HIV). In embodiments, the increased proportion is a proportion of at least about 10%, 20%, 30%, 40%, or 50%. In embodiments, detecting the diversity of microorganisms in the biological sample or a plurality of microorganisms in the biological sample includes detecting whether at least 30% of the bacteria in the biological sample are in the Prevotellaceae family of bacteria, at least 10% of the bacteria in the biological sample are in the Streptococcaceae family of bacteria, and at least 10% of the bacteria in the biological sample are in the Veillonellaceae family of bacteria.

In embodiments, detecting the diversity of microorganisms in the biological sample or a plurality of microorganisms in the biological sample includes detecting whether the biological sample has an increased proportion of bacteria in the Pseudomonadaceae family of bacteria compared to a standard control (such as the proportion in a general population of subjects who have pneumonia and are infected with HIV). In embodiments, detecting the diversity of microorganisms in the biological sample or a plurality of microorganisms in the biological sample includes detecting whether the biological sample has an increased proportion of bacteria in the Pseudomonadaceae family of bacteria compared to a standard control (such as the proportion in a general population of subjects who have pneumonia and are infected with HIV). In embodiments, the increased proportion is a proportion of at least about 10%, 20%, 30%, 40%, or 50%. In embodiments, detecting the diversity of microorganisms in the biological sample or a plurality of microorganisms in the biological sample includes detecting whether at least 40% of the bacteria in the biological sample are in the Pseudomonadaceae family of bacteria, at least 10% of the bacteria in the biological sample are in the Sphingomonadaceae family of bacteria, and at least 5% of the bacteria in the biological sample are in the Prevotellaceae family of bacteria. In embodiments, detecting the diversity of microorganisms in the biological sample or a plurality of microorganisms in the biological sample includes detecting whether the biological sample has an increased proportion of bacteria in the Streptococcaceae family of bacteria compared to a standard control (such as the proportion in general population of subjects who have pneumonia and are infected with HIV). In embodiments, detecting the diversity of microorganisms in the biological sample or a plurality of microorganisms in the biological sample includes detecting whether the biological sample has an increased proportion of bacteria in the Streptococcaceae family of bacteria compared to a standard control (such as the proportion in a general population of subjects who have pneumonia and are infected with HIV). In embodiments, the increased proportion is a proportion of at least about 10%, 20%, 30%, 40%, or 50%. In embodiments, detecting the diversity of microorganisms in the biological sample or a plurality of microorganisms in the biological sample includes detecting whether at least 40% of the bacteria in the biological sample are in the Streptococcaceae family of bacteria, at least 5% of the bacteria in the biological sample are in the Veillonellaceae family of bacteria, and at least 10% of the bacteria in the biological sample are in the Prevotellaceae family of bacteria. In embodiments, detecting the diversity of microorganisms in the biological sample or a plurality of microorganisms in the biological sample includes detecting whether the biological sample has an increased proportion of bacteria in the Prevotellaceae family of bacteria compared to a standard control (such as the proportion in a general population of subjects who have pneumonia and are infected with HIV). In embodiments, detecting the diversity of microorganisms in the biological sample or a plurality of microorganisms in the biological sample includes detecting whether the biological sample has an increased proportion of bacteria in the Prevotellaceae family of bacteria compared to a standard control (such as the proportion in a general population of subjects who have pneumonia and are infected with HIV). In embodiments, the increased proportion is a proportion of at least about 10%, 20%, 30%, 40%, or 50%. In embodiments, detecting the diversity of microorganisms in the biological sample or a plurality of microorganisms in the biological sample includes detecting whether at least 30% of the bacteria in the biological sample are in the Prevotellaceae family of bacteria, at least 10% of the bacteria in the biological sample are in the Streptococcaceae family of bacteria, and at least 10% of the bacteria in the biological sample are in the Veillonellaceae family of bacteria. In embodiments, detecting the diversity of microorganisms in the biological sample or a plurality of microorganisms in the biological sample includes detecting whether (a) the biological sample has an increased proportion of bacteria in the Pseudomonadaceae family of bacteria compared to a general population of subjects who have pneumonia and are infected with HIV; (b) the biological sample has an increased proportion of bacteria in the Pseudomonadaceae family of bacteria compared to a general population of subjects who have pneumonia and are infected with HIV, wherein the increased proportion is a proportion of at least about 10%, 20%, 30%, 40%, or 50%; (c) at least 40% of the bacteria in the biological sample are in the Pseudomonadaceae family of bacteria, at least 10% of the bacteria in the biological sample are in the Sphingomonadaceae family of bacteria, and at least 5% of the bacteria in the biological sample are in the Prevotellaceae family of bacteria; (d) the biological sample has an increased proportion of bacteria in the Streptococcaceae family of bacteria compared to a general population of subjects who have pneumonia and are infected with HIV; (e) the biological sample has an increased proportion of bacteria in the Streptococcaceae family of bacteria compared to a general population of subjects who have pneumonia and are infected with HIV, wherein the increased proportion is a proportion of at least about 10%, 20%, 30%, 40%, or 50%; (f) at least 40% of the bacteria in the biological sample are in the Streptococcaceae family of bacteria, at least 5% of the bacteria in the biological sample are in the Veillonellaceae family of bacteria, and at least 10% of the bacteria in the biological sample are in the Prevotellaceae family of bacteria; (g) the biological sample has an increased proportion of bacteria in the Prevotellaceae family of bacteria compared to a general population of subjects who have pneumonia and are infected with HIV; (h) the biological sample has an increased proportion of bacteria in the Prevotellaceae family of bacteria compared to a general population of subjects who have pneumonia and are infected with HIV, wherein the increased proportion is a proportion of at least about 10%, 20%, 30%, 40%, or 50%; and/or (i) at least 30% of the bacteria in the biological sample are in the Prevotellaceae family of bacteria, at least 10% of the bacteria in the biological sample are in the Streptococcaceae family of bacteria, and at least 10% of the bacteria in the biological sample are in the Veillonellaceae family of bacteria.

In embodiments, the biological sample is a bodily fluid.

In embodiments, the bodily fluid is blood, serum, or plasma.

In embodiments, the metabolite is a plurality of metabolites.

In embodiments, the metabolite includes any 1 of or any combination of (e.g., any combination of 2, 3, 4, 5, 6, 7, 8, 9, 10, 15, 20, 30 or more of): 4-guanidinobutanoate, cis-urocanate, trans-urocanate, xanthurenate, glucose, 1-methylnicotinamide, biopterin, 15-HETE,leukotriene B4, eicosanodioate, maleate (cis-Butenedioate), 13-HODE+9-HODE, scyllo-inositol, 1-palmitoylglycerophosphate, 1-palmitoylglycerol (1-monopalmitin), 1-stearoylglycerol (1-monostearin), 2-palmitoylglycerol (2-monopalmitin), glycerophosphoinositol, chenodeoxycholate, glycochenodeoxycholate, taurochenodeoxycholate, glycocholenate sulfate, glycodeoxycholate, glycolithocholate sulfate, glycoursodeoxycholate, taurocholenate sulfate, taurodeoxycholate, ursodeoxycholate, 21-hydroxypregnenolone disulfate, 5alpha-pregnan-3(alpha or beta),20beta-diol disulfate, pregnen-diol disulfate, N6-methyladenosine, 4-ureidobutyrate, glycylphenylalanine, glycylvaline, lysyltyrosine, phenylalanylaspartate, tyrosyllysine, thymol sulfate, 1-methylxanthine, 7-methylxanthine, Theobromine, N-acetylphenylalanine, Maltose, 1-margaroylglycerophosphoethanolamine, 1-palmitoylglycerophosphoglycerol, 2-stearoylglycerophosphoethanolamine, 5alpha-pregnan-3beta,20alpha-diol disulfate, N4-acetylcytidine, 3-methyl-2-oxobutyrate, 4-methyl-2-oxopentanoate, methionine sulfone, 1-dihomo-linolenylglycerol (alpha, gamma), 1-myristoylglycerol (1-monomyristin), inosine, phenylalanyltryptophan, phenylalanine, 1-palmitoylglycerophosphoethanolamine, glycolithocholate and alpha-ketoglutarate.

In embodiments, the metabolite is a tryptophan metabolite, an arachidonic acid metabolite, an eicosanoid, or a product of primary or secondary bile metabolism.

In embodiments, the metabolite is leukotriene B4, xanthurenate, 15-hydroxyeicosatetraenoic acid, chenodeoxycholate, glycodeoxycholate, taurochenodeoxycholate, or ursodeoxycholate.

In embodiments, the metabolite is a lysolipid metabolite, a pyrimidine metabolite, or a monoacylglycerol.

In embodiments, the metabolite is a valine metabolite, a leucine metabolite, or a monoacylglycerol associated with lipid metabolism.

In embodiments, the metabolite is 3-methyl-2-oxobutyrate, 4-methyl-2-oxopentanoate, 1-dihomo-linolenylglycerol, 1-myristoylglycerol, or inosine.

In embodiments, the method further includes detecting at least one metabolite in an additional biological sample from the subject. In embodiments, the method further includes detecting (i) the diversity of microorganisms in an additional biological sample from the subject and/or (ii) a plurality of microorganisms in an additional biological sample from the subject.

In embodiments, the additional biological sample is a bronchoalveolar lavage (BAL) sample, sputum, phlegm, saliva, or mucus.

In embodiments, the method further includes identifying the subject as an increased risk of dying within about 1, 2, 3, 4, 5, 6, 7, 8, 9, or 10 weeks compared to the general population if the subject has an increased level of expression of 1 of or any combination of 2, 3, 4, 5, 6, 7, 8, 9, 10, 11, 12, 13, 14, 15, or 16 of IFNγ, IFNα, TNFα, MUC5AC, IL-17A, IL-4, IL-5, IL-13, IL-33, OCEL1, CCL11, FOXP3, PD-1, CD45RO, CD39, and/or GAPDH compared to the general population. In embodiments, the method further includes identifying the subject as an increased risk of dying within about 1, 2, 3, 4, 5, 6, 7, 8, 9, or 10 weeks compared to the general population if the subject hasan increased level of 1, 2, 3, or 5 or 3-methyl-2-oxobutyrate, 4-methyl-2-oxopentanoate, 1-dihomo-linolenylglycerol, 1-myristoylglycerol, or inosine compared to the general population. In embodiments, the method further includes identifying the subject as an increased risk of dying within about 1, 2, 3, 4, 5, 6, 7, 8, 9, or 10 weeks compared to the general population if the subject has an increased proportion of lung microbiome bacteria in the Prevotellaceae family of bacteria compared to the general population. In embodiments, the method further includes identifying the subject as an increased risk of dying within about 1, 2, 3, 4, 5, 6, 7, 8, 9, or 10 weeks compared to the general population if the subject has an increased proportion of lung microbiome bacteria in the Prevotellaceae family of bacteria compared to the general population, wherein the increased proportion is a proportion of at least about 10%, 20%, 30%, 40%, or 50%. In embodiments, the method further includes identifying the subject as an increased risk of dying within about 1, 2, 3, 4, 5, 6, 7, 8, 9, or 10 weeks compared to the general population if the subject has a proportion of at least 30% of lung microbiome bacteria in the Prevotellaceae family of bacteria, at least 10% of lung microbiome bacteria in the Streptococcaceae family of bacteria, and at least 10% of lung microbiome bacteria in the Veillonellaceae family of bacteria. In embodiments, the method further includes identifying the subject as an increased risk of dying within about 1, 2, 3, 4, 5, 6, 7, 8, 9, or 10 weeks compared to the general population if the subject (a) has an increased level of expression of 1 of or any combination of 2, 3, 4, 5, 6, 7, 8, 9, 10, 11, 12, 13, 14, 15, or 16 of IFNγ, IFNα, TNFα, MUC5AC, IL-17A, IL-4, IL-5, IL-13, IL-33, OCEL1, CCL11, FOXP3, PD-1, CD45RO, CD39, and/or GAPDH compared to the general population; (b) has an increased level of 1, 2, 3, or 5 or 3-methyl-2-oxobutyrate, 4-methyl-2-oxopentanoate, 1-dihomo-linolenylglycerol, 1-myristoylglycerol, or inosine compared to the general population; (c) has an increased proportion of lung microbiome bacteria in the Prevotellaceae family of bacteria compared to the general population; (d) has an increased proportion of lung microbiome bacteria in the Prevotellaceae family of bacteria compared to the general population, wherein the increased proportion is a proportion of at least about 10%, 20%, 30%, 40%, or 50%; and/or (e) has a proportion of at least 30% of lung microbiome bacteria in the Prevotellaceae family of bacteria, at least 10% of lung microbiome bacteria in the Streptococcaceae family of bacteria, and at least 10% of lung microbiome bacteria in the Veillonellaceae family of bacteria.

In embodiments, the method further includes identifying the subject as an increased risk of dying within about 1, 2, 3, 4, 5, 6, 7, 8, 9, or 10 weeks compared to the general population if the subject is identified as having a Mycobacterium sp. infection, an Aspergillus sp. infection, or an increased risk thereof.

In embodiments, the Mycobacterium sp. is M. tuberculosis.

In embodiments, the Aspergillus sp. is A. fumigatus or A. flavus.

In embodiments, a biological sample is a bodily fluid obtained by filtration and/or centrifugation. For example, the biological sample may be a filtrate of e.g., blood, a BAL sample, sputum, phlegm, saliva, or mucus, or the supernatant of a centrifuged BAL sample or centrifuged blood, sputum, phlegm, saliva, or mucus. In embodiments, a filtrate is centrifuged. In embodiments a supernatant is filtered. In embodiments, centrifugation is used to increase the passage of a fluid through a filter. Non-limiting examples of filters include filters that restrict any molecule greater than, e.g., 50, 100, 200, 300, 400, 500, 50-500, 50-100, 100-500 nm in diameter (or average diameter), or greater than 0.5, 1, 1.5, 2, 2.5, 5, 10, 15, 25, 50, 100, or 200 microns in diameter (e.g., average diameter). In embodiments, a filter has pores of about 50, 100, 200, 300, 400, 500, 50-500, 50-100, 100-500 nm in diameter or about 0.5, 1, 1.5, 2, 2.5, 5, 10, 15, 25, 50, 100, or 200 microns in diameter.

In embodiments, detecting a compound (e.g., a metabolite) and/or the expression level thereof includes Ultrahigh Performance Liquid Chromatography-Tandem Mass Spectrometry (UPLC-MS/MS), Gas Chromatography-Mass Spectrometry (GC-MS), high performance liquid chromatography (HPLC), gas chromatography, liquid chromatography, Mass spectrometry (MS), inductively coupled plasma-mass spectrometry (ICP-MS), accelerator mass spectrometry (AMS), thermal ionization-mass spectrometry (TIMS) and spark source mass spectrometry (SSMS), matrix-assisted laser desorption/ionization (MALDI), and/or MALDI-TOF.

In embodiments, detecting the expression level of a protein includes assaying the level of the compound (e.g., with high-performance liquid chromatography (HPLC), liquid chromatography-mass spectrometry (LC/MS), an enzyme-linked immunosorbent assay (ELISA), protein immunoprecipitation, immunoelectrophoresis, protein immunostaining, and/or Western blot) or the level of mRNA that encodes the protein. In embodiments, detecting the expression level of a compound (e.g., a protein) includes lysing a cell. In embodiments, detecting the expression level of a compound includes a polymerase chain reaction (e.g., reverse transcriptase polymerase chain reaction), RNA sequencing microarray analysis, immunohistochemistry, or flow cytometry.

In an aspect, provided herein is a kit or system for performing a diagnostic method disclosed herein. In embodiments, the kit or system includes one or more primers, probes, or antibodies specific for a protein or any combination of any one of the proteins mentioned herein. In embodiments, the kit or system includes one or more primers or probes specific for the mRNA of a protein or any combination of any one of the proteins mentioned herein. In embodiments, the kit or system includes one or more primers or probes specific for one or more of any combination of the bacterial species, genera, families or other taxa, disclosed herein. In embodiments, the one or more probes or primers hybridize the 16S rRNA gene of one or more bacterial taxa disclosed herein under stringent hybridization conditions.

### IV. Methods of Treatment and Monitoring

In an aspect, method of treating or preventing a lung infection in a subject in need thereof is provided. In embodiments, the method includes administering an effective amount of at least one antibiotic or antifungal agent to the subject. In embodiments, the subject (a) has an increased level of expression of 1 of or any combination of 2, 3, 4, 5, 6, 7, 8, 9, 10, 11, 12, 13, 14, 15, or 16 of IFNγ, IFNα, TNFα, MUC5AC, IL-17A, IL-4, IL-5, IL-13, IL-33, OCEL1, CCL11, FOXP3, PD-1, CD45RO, CD39, and/or GAPDH compared to a general or healthy population; (b) has an increased level of 1, 2, 3, or 5 or 3-methyl-2-oxobutyrate, 4-methyl-2-oxopentanoate, 1-dihomo-linolenylglycerol, 1-myristoylglycerol, or inosine compared to a general or healthy population of subjects; (c) has an increased proportion of lung microbiome bacteria in the Prevotellaceae family of bacteria compared to a healthy or general population of subjects; (d) has an increased proportion of lung microbiome bacteria in the Prevotellaceae family of bacteria compared to a healthy or general population of subjects, wherein the increased proportion is a proportion of at least about 10%, 20%, 30%, 40%, or 50%; (e) has a proportion of at least 30% of lung microbiome bacteria in the Prevotellaceae family of bacteria, at least 10% lung microbiome bacteria in the Streptococcaceae family of bacteria, and at least 10% lung microbiome bacteria in the Veillonellaceae family of bacteria; (f) has increased TIM-3 expression compared to a general or healthy population of subjects; (g) has an increased level of 1, 2, 3, 4, 5, 6, or 7 of leukotriene B4, xanthurenate, 15-hydroxyeicosatetraenoic acid, chenodeoxycholate, glycodeoxycholate, taurochenodeoxycholate, and/or ursodeoxycholate compared to a general or healthy population of subjects; (h) has an increased proportion of lung microbiome bacteria in the Pseudomonadaceae family of bacteria compared to a healthy or general population of subjects; (i) has an increased proportion of lung microbiome bacteria in the Pseudomonadaceae family of bacteria compared to a healthy or general population of subjects, wherein the increased proportion is a proportion of at least about 10%, 20%, 30%, 40%, or 50%; and/or (j) has a proportion of at least 40% of lung microbiome bacteria in the Pseudomonadaceae family of bacteria, at least 10% of lung microbiome bacteria in the Sphingomonadaceae family of bacteria, and at least 5% of lung microbiome bacteria in the Prevotellaceae family of bacteria.

In an aspect, a method of treating or preventing a lung infection in a subject in need thereof us provided. In embodiments, the method includes (a) detecting (i) an airway microbiome; (ii) the diversity of microorganisms; (iii) a plurality of microorganisms; and/or (iv) at least one metabolite, in a biological sample from the subject; and (b) administering to the subject an effective amount of at least one antibiotic or antifungal agent.

In an aspect, a method of treating or preventing a Mycobacterium sp. infection in a subject in need thereof is provided. In embodiments, the subject has been identified as having a Mycobacterium sp. infection according to a method provided herein. In embodiments, the subject has been identified as having an increased risk for a Mycobacterium sp. infection according to a method provided herein.

In embodiments, the method includes administering an effective amount of at least one antibiotic compound to the subject.

In an aspect, a method of monitoring a subject for a Mycobacterium sp. infection is provided. In embodiments, the subject has been identified as having an increased risk for a Mycobacterium sp. infection according to a method provided herein. In embodiments, the subject is monitored for the infection more frequently than subjects who are not identified as having an increased risk for a Mycobacterium sp. infection according to a method provided herein.

In embodiments, the subject has increased TIM-3 expression compared to a standard control (such as the level of expression in a general or healthy population of subjects). In embodiments, the subject has an increased level of any 1 of or any combination of 2, 3, 4, 5, 6, or 7 of leukotriene B4, xanthurenate, 15-hydroxyeicosatetraenoic acid, chenodeoxycholate, glycodeoxycholate, taurochenodeoxycholate, and/or ursodeoxycholate compared to a standard control (such as a general or healthy population of subjects). In embodiments, the subject has an increased proportion of lung microbiome bacteria in the Pseudomonadaceae family of bacteria compared to a standard control (such as a healthy or general population of subjects). In embodiments, the subject has an increased proportion of lung microbiome bacteria in the Pseudomonadaceae family of bacteria compared to a standard control (such as the proportion in a healthy or general population of subjects). In embodiments, the increased proportion is a proportion of at least about 10%, 20%, 30%, 40%, or 50%. In embodiments, the subject has a proportion of at least 40% of lung microbiome bacteria in the Pseudomonadaceae family of bacteria, at least 10% of lung microbiome bacteria in the Sphingomonadaceae family of bacteria, and at least 5% of lung microbiome bacteria in the Prevotellaceae family of bacteria. In embodiments, the general population is a general population of subjects who have pneumonia and are infected with HIV.

In an aspect, a method of treating or preventing a Mycobacterium sp. infection in a subject in need thereof is provided. In embodiments, the method includes (a) detecting (i) the diversity of microorganisms; (ii) a plurality of microorganisms; and/or (iii) at least one metabolite, in a biological sample from the subject; and (b) administering to the subject an effective amount of at least one antibiotic compound.

In embodiments, the at least one antibiotic compound is 1, 2, 3, or 4 of any combination of isoniazid, rifampin, ethambutol, and/or pyrazinamide.

In embodiments, the at least one antibiotic compound is isoniazid, rifampin, and pyrazinamide.

In embodiments, the at least one antibiotic compound is isoniazid and rifampin.

In embodiments, the at least one antibiotic compound is an aminoglycoside, a fluoroquinolone, a polypeptide, a thioamide, a cycloserine, and/or p-aminosalicylic acid.

In an aspect, a method of treating or preventing an Aspergillus sp. infection in a subject in need thereof is provided. In embodiments, the subject has been identified as having a Aspergillus sp. infection according to a method provided herein. In embodiments, the subject has been identified as having an increased risk for a Aspergillus sp. infection according to a method provided herein.

In embodiments, the method includes administering an effective amount of at least one antibiotic compound to the subject.

In an aspect, a method of monitoring a subject for a Aspergillus sp. infection is provided. In embodiments, the subject has been identified as having an increased risk for a Aspergillus sp. infection according to a method provided herein. In embodiments, the subject is monitored for the infection more frequently than subjects who are not identified as having an increased risk for a Aspergillus sp. infection according to a method provided herein.

In embodiments, the method includes administering an effective amount of at least one antifungal agent to the subject. In embodiments, the subject has an increased level of expression of any 1 of or any combination of 2, 3, 4, 5, 6, 7, 8, 9, 10, 11, 12, 13, 14, 15, or 16 of IFNγ, IFNα, TNFα, MUC5AC, IL-17A, IL-4, IL-5, IL-13, IL-33, OCEL1, CCL11, FOXP3, PD-1, CD45RO, CD39, and/or GAPDH compared to a standard control (such as the level in a general or healthy population). In embodiments, the subject has an increased level of 1, 2, 3, or 5 or 3-methyl-2-oxobutyrate, 4-methyl-2-oxopentanoate, 1-dihomo-linolenylglycerol, 1-myristoylglycerol, or inosine compared to a standard control (such as the level in a general or healthy population of subjects). In embodiments, the subject has an increased proportion of lung microbiome bacteria in the Prevotellaceae family of bacteria compared to a standard control (such as a healthy or general population of subjects). In embodiments, the subject has an increased proportion of lung microbiome bacteria in the Prevotellaceae family of bacteria compared to a standard control (such as healthy or general population of subjects). In embodiments, the increased proportion is a proportion of at least about 10%, 20%, 30%, 40%, or 50%. In embodiments, the subject has a proportion of at least 30% of lung microbiome bacteria in the Prevotellaceae family of bacteria, at least 10% lung microbiome bacteria in the Streptococcaceae family of bacteria, and at least 10% lung microbiome bacteria in the Veillonellaceae family of bacteria. In embodiments, the general population is a general population of subjects who have pneumonia and are infected with HIV.

In an aspect, a method of treating or preventing an Aspergillus sp. infection in a subject in need thereof is provided. In embodiments, the method includes (a) detecting (i) the diversity of microorganisms; (ii) a plurality of microorganisms; and/or (iii) at least one metabolite, in a biological sample from the subject; and (b) administering to the subject an effective amount of at least one antifungal agent.

In embodiments, the at least one antifungal agent is a triazole antifungal agent.

In embodiments, the at least one antifungal agent is 1, 2, 3, 4, 5, 6, or 7 of any combination of amphotericin B, liposomal amphotericin B, voriconazole, caspofungin, flucytosine, itraconazole, and/or posaconazole.

In embodiments, a method herein further includes directing a subject identified as having or at risk of a Mycobacterium sp. infection to obtain treatment or additional screening for a Mycobacterium sp. infection.

In embodiments, a method herein further includes treating a subject identified as having or at risk of a Mycobacterium sp. infection for a Mycobacterium sp. infection.

In embodiments, a method herein further includes directing a subject identified as having or at risk of an Aspergillus sp. infection to obtain treatment or additional screening for an Aspergillus sp. infection.

In embodiments, a method herein further includes treating a subject identified as having or at risk of an Aspergillus sp. infection for a Aspergillus sp. infection.

In embodiments, the Mycobacterium sp. is M. tuberculosis.

In embodiments, the Aspergillus sp. is A. fumigatus or A. flavus.

In embodiments, bacteria may be differentiated at, e.g., the family level, the genus level, the species, level, the sub-species level, the strain level or by any other taxonomic method described herein and otherwise known in the art.

In embodiments, a subject is administered an effective amount of one or more compounds (e.g., therapeutic compounds). The terms effective amount and effective dosage are used interchangeably. The term effective amount is defined as any amount necessary to produce a desired physiologic response (e.g., reduction of inflammation, infection, or dysbiosis). In embodiments, an effective amount is an amount sufficient to accomplish a stated purpose (e.g. achieve the effect for which it is administered, treat a disease, kill pathogenic cells, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” Effective amounts and schedules for administering the agent may be determined empirically by one skilled in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)).

For prophylactic use, a therapeutically effective amount of a compound (such as an antibiotic or antifungal compound) described herein is administered to a subject prior to or during early onset (e.g., upon initial signs and symptoms of) an infection. In embodiments, therapeutic treatment involves administering to a subject a therapeutically effective amount of an agent described herein after diagnosis or development of disease. Thus, in embodiments, a method of treating a disease (e.g., an inflammatory disease, an infection, or dysbiosis) in a subject in need thereof is provided.

The terms “subject,” “patient,” “individual,” etc. are not intended to be limiting and can be generally interchanged. In embodiments, an individual described as a “patient” does not necessarily have a given disease, but may, e.g., be merely seeking medical advice.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. In embodiments, treatment can refer to a 10%, 20%, 30%, 40%, 50%, 60%, 70%, 80%, 90%, or 100% reduction in the severity of an established disease, condition, or symptom of the disease or condition (e.g., infection or dysbiosis). For example, a method for treating a disease is considered to be a treatment if there is a 10% reduction in one or more symptoms of the disease in a subject as compared to a control. In embodiments, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

Regardless of how the compositions are formulated, the dosage required will depend on the route of administration, the nature of the formulation, the nature of the subject's condition, e.g., immaturity of the immune system or a gastrointestinal disorder, the subject's size, weight, surface area, age, and sex, other drugs being administered, and the judgment of the attending clinicians.

Administrations can be single or multiple (e.g., 2- or 3-, 4-, 6-, 8-, 10-, 20-, 50-, 100-, 150-, or more fold). The duration of treatment with any composition provided herein can be any length of time from as short as one day to as long as the life span of the host (e.g., many years). For example, a composition can be administered 1, 2, 3, 4, 5, 6, or 7 times a week (for, for example, 4 weeks to many months or years); once a month (for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

The compositions may also be administered in conjunction with other therapeutic agents. Other therapeutic agents will vary according to the particular disorder, but can include, for example, dysbiosis or an infection. Concurrent administration of two or more therapeutic agents does not require that the agents be administered at the same time or by the same route, as long as there is an overlap in the time period during which the agents are exerting their therapeutic effect. Simultaneous or sequential administration is contemplated, as is administration on different days or weeks.

A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of a disease, pathology, or condition, or their symptoms. The full prophylactic effect does not necessarily occur by administration of one dose, and may occur only after administration of a series of doses. Thus, a prophylactically effective amount may be administered in one or more administrations. Guidance can be found in the literature for appropriate dosages for given classes of pharmaceutical products. For example, for the given parameter, an effective amount will show an increase (e.g., improvement of function, such as lung function) or decrease (e.g., reduction of a symptom) of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

In embodiments, a compound is administered in a composition that includes a pharmaceutically acceptable excipient. “Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

### EMBODIMENTS

Embodiments include P1 to P55 following.

### Embodiment P1

A method of detecting whether a subject has or is at risk of an Aspergillus sp. or a Mycobacterium sp. infection, comprising:

(a) obtaining a biological sample from the subject; and

(b) detecting (i) a diversity of microorganisms in the biological sample; and/or (ii) a plurality of microorganisms in the biological sample.

### Embodiment P2

The method of embodiment P1, wherein the subject is infected with human immunodeficiency virus (HIV).

### Embodiment P3

The method of embodiment P1 or P2, wherein the subject has pneumonia.

### Embodiment P4

The method of embodiment P3, wherein the pneumonia is bacterial pneumonia.

### Embodiment P5

The method of any one of embodiments P1-P4, wherein the subject has tuberculosis (TB).

### Embodiment P6

The method of any one of embodiments P1-P5, wherein the subject has TB pneumonia.

### Embodiment P7

The method of any one of embodiments P1-P6, wherein the subject has been administered an antibiotic.

### Embodiment P8

The method of embodiment P7, wherein the antibiotic is ceftriaxone.

### Embodiment P9

The method of any one of embodiments P1-P8, wherein the biological sample is a bronchoalveolar lavage (BAL) sample, sputum, phlegm, saliva, or mucus.

### Embodiment P10

The method of any one of embodiments P1-P9, wherein the microorganisms are bacterial microorganisms.

### Embodiment P11

The method of any one of embodiments P1-P10, wherein detecting the diversity of microorganisms in the biological sample comprises characterizing the microbiome in the biological sample.

### Embodiment P12

The method of any one of embodiments P1-P11, wherein detecting the diversity of microorganisms in the biological sample or a plurality of microorganisms in the biological sample comprises amplifying and sequencing 16S rRNA genes of microorganisms in the sample.

### Embodiment P13

The method of embodiment P12, wherein detecting the diversity of microorganisms in the biological sample or a plurality of microorganisms in the biological sample comprises amplifying and sequencing the V4 region of 16S rRNA genes of microorganisms in the sample.

### Embodiment P14

The method of any one of embodiments P1-P13, wherein the microorganisms are bacterial microorganisms, and wherein detecting the diversity of microorganisms in the biological sample or a plurality of microorganisms in the biological sample comprises detecting bacteria, or a proportion of bacteria, that are in the family Prevotellaceae, Pseudomonadaceae, Sphingomonadaceae, Streptococcaceae, and/or Veillonellaceae.

### Embodiment P15

The method of any one of embodiments P1-P14, further comprising determining the expression level of at least one immune gene in the subject.

### Embodiment P16

The method of embodiment P15, wherein the at least one immune gene is 1 of or any combination of 2, 3, 4, 5, 6, 7, 8, 9, 10, 11, 12, 13, 14, 15, 16, 17, 18, 19, or 20 of IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH.

### Embodiment P17

The method of embodiment P16, wherein detecting the diversity of microorganisms in the biological sample comprises determining the number of families, the number of genera, the number of species, the Faith's Phylogenetic Diversity, the Shannon Diversity, and/or the Simpson Diversity of the microorganisms.

### Embodiment P18

The method of any one of embodiments P1-P17, wherein the subject has pneumonia and is infected with HIV, and wherein detecting whether the subject is at risk of an Aspergillus sp. or a Mycobacterium sp. infection comprises detecting an increased risk of an Aspergillus sp. or Mycobacterium sp. infection compared to a general population of subjects who have pneumonia and are infected with HIV.

### Embodiment P19

The method of embodiment P18, wherein detecting the diversity of microorganisms in the biological sample or a plurality of microorganisms in the biological sample comprises detecting whether:
(a) the biological sample has an increased proportion of bacteria in the Pseudomonadaceae family of bacteria compared to a general population of subjects who have pneumonia and are infected with HIV;
(b) the biological sample has an increased proportion of bacteria in the Pseudomonadaceae family of bacteria compared to a general population of subjects who have pneumonia and are infected with HIV, wherein the increased proportion is a proportion of at least about 10%, 20%, 30%, 40%, or 50%;
(c) at least 40% of the bacteria in the biological sample are in the Pseudomonadaceae family of bacteria, at least 10% of the bacteria in the biological sample are in the Sphingomonadaceae family of bacteria, and at least 5% of the bacteria in the biological sample are in the Prevotellaceae family of bacteria;
(d) the biological sample has an increased proportion of bacteria in the Streptococcaceae family of bacteria compared to a general population of subjects who have pneumonia and are infected with HIV;
(e) the biological sample has an increased proportion of bacteria in the Streptococcaceae family of bacteria compared to a general population of subjects who have pneumonia and are infected with HIV, wherein the increased proportion is a proportion of at least about 10%, 20%, 30%, 40%, or 50%;
(f) at least 40% of the bacteria in the biological sample are in the Streptococcaceae family of bacteria, at least 5% of the bacteria in the biological sample are in the Veillonellaceae family of bacteria, and at least 10% of the bacteria in the biological sample are in the Prevotellaceae family of bacteria;
(g) the biological sample has an increased proportion of bacteria in the Prevotellaceae family of bacteria compared to a general population of subjects who have pneumonia and are infected with HIV;
(h) the biological sample has an increased proportion of bacteria in the Prevotellaceae family of bacteria compared to a general population of subjects who have pneumonia and are infected with HIV, wherein the increased proportion is a proportion of at least about 10%, 20%, 30%, 40%, or 50%; and/or
(i) at least 30% of the bacteria in the biological sample are in the Prevotellaceae family of bacteria, at least 10% of the bacteria in the biological sample are in the Streptococcaceae family of bacteria, and at least 10% of the bacteria in the biological sample are in the Veillonellaceae family of bacteria.

### Embodiment P20

The method of any one of embodiments P1-P19, further comprising obtaining an additional biological sample from the subject, and detecting at least one metabolite in the additional biological sample.

### Embodiment P21

The method of embodiment P20, wherein the additional biological sample is a bodily fluid.

### Embodiment P22

The method of embodiment P21, wherein the bodily fluid is blood, serum, or plasma.

### Embodiment P23

A method of detecting whether a subject has or is at risk of an Aspergillus sp. or a Mycobacterium sp. infection, comprising:
(a) obtaining a biological sample from the subject; and
(b) detecting at least one metabolite in the biological sample.

### Embodiment P24

The method of embodiment P23, wherein the subject
(a) is infected with human immunodeficiency virus (HIV);
(b) has pneumonia;
(c) has bacterial pneumonia
(d) has TB;
(e) has TB pneumonia;
(f) has been administered an antibiotic; and/or
(g) has been administered ceftriaxone.

### Embodiment P25

The method of any one of embodiments P1-P24, wherein the biological sample is a bodily fluid.

### Embodiment P26

The method of any one of embodiments P1-P25, wherein the bodily fluid is blood, serum, or plasma.

### Embodiment P27

The method of any one of embodiments P20-P26, wherein the metabolite is a plurality of metabolites.

### Embodiment P28

The method of any one of embodiments P20-P27, wherein the metabolite is a tryptophan metabolite, an arachidonic acid metabolite, an eicosanoid, or a product of primary or secondary bile metabolism.

### Embodiment P29

The method of any one of embodiments P20-P28, wherein the metabolite is leukotriene B4, xanthurenate, 15-hydroxyeicosatetraenoic acid, chenodeoxycholate, glycodeoxycholate, taurochenodeoxycholate, or ursodeoxycholate.

### Embodiment P30

The method of any one of embodiments P20-P29, wherein the metabolite is a lysolipid metabolite, a pyrimidine metabolite, or a monoacylglycerol.

### Embodiment P31

The method of any one of embodiments P20-P30, wherein the metabolite is a valine metabolite, a leucine metabolite, or a monoacylglycerol associated with lipid metabolism.

### Embodiment P32

The method of any one of embodiments P20-P31, wherein the metabolite is 3-methyl-2-oxobutyrate, 4-methyl-2-oxopentanoate, 1-dihomo-linolenylglycerol, 1-myristoylglycerol, or inosine.

### Embodiment P33

The method of any one of embodiments P20-P32, further comprising obtaining an additional biological sample from the subject, and detecting (i) the diversity of microorganisms in the additional biological sample and/or (ii) a plurality of microorganisms in the biological sample in the additional biological sample.

### Embodiment P34

The method of embodiment P33, wherein the additional biological sample is a bronchoalveolar lavage (BAL) sample, sputum, phlegm, saliva, or mucus.

### Embodiment P35

The method of any one of embodiments P1-P34, further comprising identifying the subject as having or at risk of a Mycobacterium sp. infection if the subject:
(a) has increased TIM-3 expression compared to a general or healthy population of subjects;
(b) has an increased level of 1, 2, 3, 4, 5, 6, or 7 of leukotriene B4, xanthurenate, 15-hydroxyeicosatetraenoic acid, chenodeoxycholate, glycodeoxycholate, taurochenodeoxycholate, and/or ursodeoxycholate compared to a general or healthy population of subjects;
(c) has an increased proportion of lung microbiome bacteria in the Pseudomonadaceae family of bacteria compared to a healthy or general population of subjects; (d) has an increased proportion of lung microbiome bacteria in the Pseudomonadaceae family of bacteria compared to a healthy or general population of subjects, wherein the increased proportion is a proportion of at least about 10%, 20%, 30%, 40%, or 50%; and/or
(e) has a proportion of at least 40% of lung microbiome bacteria in the Pseudomonadaceae family of bacteria, at least 10% of lung microbiome bacteria in the Sphingomonadaceae family of bacteria, and at least 5% of lung microbiome bacteria in the Prevotellaceae family of bacteria.

### Embodiment P36

The method of any one of embodiments P1-P34, further comprising identifying the subject as at risk of an Aspergillus sp. infection if the subject:
(a) has an increased level of expression of 1 of or any combination of 2, 3, 4, 5, 6, 7, 8, 9, 10, 11, 12, 13, 14, 15, or 16 of IFNγ, IFNα, TNFα, MUC5AC, IL-17A, IL-4, IL-5, IL-13, IL-33, OCEL1, CCL11, FOXP3, PD-1, CD45RO, CD39, and/or GAPDH compared to a general or healthy population;
(b) has an increased level of 1, 2, 3, or 5 or 3-methyl-2-oxobutyrate, 4-methyl-2-oxopentanoate, 1-dihomo-linolenylglycerol, 1-myristoylglycerol, or inosine compared to a general or healthy population of subjects;
(c) has an increased proportion of lung microbiome bacteria in the Prevotellaceae family of bacteria compared to a healthy or general population of subjects;
(d) has an increased proportion of lung microbiome bacteria in the Prevotellaceae family of bacteria compared to a healthy or general population of subjects, wherein the increased proportion is a proportion of at least about 10%, 20%, 30%, 40%, or 50%; and/or
(e) has a proportion of at least 30% of lung microbiome bacteria in the Prevotellaceae family of bacteria, at least 10% lung microbiome bacteria in the Streptococcaceae family of bacteria, and at least 10% lung microbiome bacteria in the Veillonellaceae family of bacteria.

### Embodiment P37

The method of embodiment P35, further comprising directing the subject to obtain treatment or additional screening for a Mycobacterium sp. infection.

### Embodiment P38

The method of embodiment P35, further comprising treating the subject for a Mycobacterium sp. infection.

### Embodiment P39

The method of embodiment P36, further comprising directing the subject to obtain treatment or additional screening for a Aspergillus sp. infection.

### Embodiment P40

The method of embodiment P36, further comprising treating the subject for a Aspergillus sp. infection.

### Embodiment P41

The method of any one of embodiments P35-P40, wherein the general population is a general population of subjects who have pneumonia and are infected with HIV.

### Embodiment P42

A method of detecting whether a subject who has pneumonia and is infected with HIV has an increased risk of dying within about 1, 2, 3, 4, 5, 6, 7, 8, 9, or 10 weeks compared to a general population of subjects who have pneumonia and are infected with HIV comprising:
(a) obtaining at least one biological sample from the subject; and/or
(b) detecting (i) the diversity of microorganisms in the at least one biological sample; (ii) a plurality of microorganisms in the at least one biological sample; and/or (iii) at least one metabolite in the at least one biological sample.

### Embodiment P43

The method of embodiment P42, further comprising identifying the subject as an increased risk of dying within about 1, 2, 3, 4, 5, 6, 7, 8, 9, or 10 weeks compared to the general population if the subject:
(a) has an increased level of expression of 1 of or any combination of 2, 3, 4, 5, 6, 7, 8, 9, 10, 11, 12, 13, 14, 15, or 16 of IFNγ, IFNα, TNFα, MUC5AC, IL-17A, IL-4, IL-5, IL-13, IL-33, OCEL1, CCL11, FOXP3, PD-1, CD45RO, CD39, and/or GAPDH compared to the general population;
(b) has an increased level of 1, 2, 3, or 5 or 3-methyl-2-oxobutyrate, 4-methyl-2-oxopentanoate, 1-dihomo-linolenylglycerol, 1-myristoylglycerol, or inosine compared to the general population;
(c) has an increased proportion of lung microbiome bacteria in the Prevotellaceae family of bacteria compared to the general population;
(d) has an increased proportion of lung microbiome bacteria in the Prevotellaceae family of bacteria compared to the general population, wherein the increased proportion is a proportion of at least about 10%, 20%, 30%, 40%, or 50%; and/or
(e) has a proportion of at least 30% of lung microbiome bacteria in the Prevotellaceae family of bacteria, at least 10% of lung microbiome bacteria in the Streptococcaceae family of bacteria, and at least 10% of lung microbiome bacteria in the Veillonellaceae family of bacteria.

### Embodiment P44

A method of treating a Mycobacterium sp. infection in a subject in need thereof, the method comprising administering an effective amount of at least one antibiotic compound to the subject, wherein the subject
(a) has increased TIM-3 expression compared to a general or healthy population of subjects;
(b) has an increased level of 1, 2, 3, 4, 5, 6, or 7 of leukotriene B4, xanthurenate, 15-hydroxyeicosatetraenoic acid, chenodeoxycholate, glycodeoxycholate, taurochenodeoxycholate, and/or ursodeoxycholate compared to a general or healthy population of subjects;
(c) has an increased proportion of lung microbiome bacteria in the Pseudomonadaceae family of bacteria compared to a healthy or general population of subjects;
(d) has an increased proportion of lung microbiome bacteria in the Pseudomonadaceae family of bacteria compared to a healthy or general population of subjects, wherein the increased proportion is a proportion of at least about 10%, 20%, 30%, 40%, or 50%; and/or
(e) has a proportion of at least 40% of lung microbiome bacteria in the Pseudomonadaceae family of bacteria, at least 10% of lung microbiome bacteria in the Sphingomonadaceae family of bacteria, and at least 5% of lung microbiome bacteria in the Prevotellaceae family of bacteria.

### Embodiment P45

A method of treating a Mycobacterium sp. infection in a subject in need thereof, the method comprising:
(a) detecting (i) the diversity of microorganisms; (ii) a plurality of microorganisms; and/or (iii) at least one metabolite, in the at least one biological sample from the subject; and
(b) administering to the subject an effective amount of at least one antibiotic compound.

### Embodiment P46

The method of embodiment P44 or P45, wherein the at least one antibiotic compound is 1, 2, 3, or 4 of any combination of isoniazid, rifampin, ethambutol, and/or pyrazinamide.

### Embodiment P47

The method of embodiment P44 or P45, wherein the at least one antibiotic compound is isoniazid, rifampin, and pyrazinamide.

### Embodiment P48

The method of embodiment P44 or P45, wherein the at least one antibiotic compound is isoniazid and rifampin.

### Embodiment P49

The method of embodiment P44 or P45, wherein the at least one antibiotic compound is an aminoglycoside, a fluoroquinolone, a polypeptide, a thioamide, a cycloserine, and/or p-aminosalicylic acid.

### Embodiment P50

A method of treating an Aspergillus sp. infection in a subject in need thereof, the method comprising administering an effective amount of at least one antifungal agent to the subject, wherein the subject
(a) has an increased level of expression of 1 of or any combination of 2, 3, 4, 5, 6, 7, 8, 9, 10, 11, 12, 13, 14, 15, or 16 of IFNγ, IFNα, TNFα, MUCSAC, IL-17A, IL-4, IL-5, IL-13, IL-33, OCEL1, CCL11, FOXP3, PD-1, CD45RO, CD39, and/or GAPDH compared to a general or healthy population;
(b) has an increased level of 1, 2, 3, or 5 or 3-methyl-2-oxobutyrate, 4-methyl-2-oxopentanoate, 1-dihomo-linolenylglycerol, 1-myristoylglycerol, or inosine compared to a general or healthy population of subjects;
(c) has an increased proportion of lung microbiome bacteria in the Prevotellaceae family of bacteria compared to a healthy or general population of subjects;
(d) has an increased proportion of lung microbiome bacteria in the Prevotellaceae family of bacteria compared to a healthy or general population of subjects, wherein the increased proportion is a proportion of at least about 10%, 20%, 30%, 40%, or 50%; and/or
(e) has a proportion of at least 30% of lung microbiome bacteria in the Prevotellaceae family of bacteria, at least 10% lung microbiome bacteria in the Streptococcaceae family of bacteria, and at least 10% lung microbiome bacteria in the Veillonellaceae family of bacteria.

### Embodiment P51

A method of treating an Aspergillus sp. infection in a subject in need thereof, the method comprising:
(a) detecting (i) the diversity of microorganisms; (ii) a plurality of microorganisms; and/or (iii) at least one metabolite, in the at least one biological sample from the subject; and
(b) administering to the subject an effective amount of at least one antifungal agent.

### Embodiment P52

The method of embodiment P50 or P51, wherein the at least one antifungal agent is a triazole antifungal agent.

### Embodiment P53

The method of embodiment P50 or P51, wherein the at least one antifungal agent is 1, 2, 3, 4, 5, 6, or 7 of any combination of amphotericin B, liposomal amphotericin B, voriconazole, caspofungin, flucytosine, itraconazole, and/or posaconazole.

### Embodiment P54

The method of any one of embodiments P1-P50, wherein the Mycobacterium sp. is M. tuberculosis.

### Embodiment P55

The method of any one of embodiments 1-51, wherein the Aspergillus sp. is A. fumigatus or A. flavus.

Additional embodiments include Embodiments 1 to 61 following:

### Embodiment 1

A method of detecting an airway microbiome in a subject who has or is suspected of having a lung infection, the method comprising detecting bacteria, or a proportion of bacteria, in a biological sample from the subject that are in the family Prevotellaceae, Pseudomonadaceae, Sphingomonadaceae, Streptococcaceae, and/or Veillonellaceae.

### Embodiment 2

The method of Embodiment 1, further comprising detecting the diversity of microorganisms in the biological sample.

### Embodiment 3

The method of Embodiment 1 or 2, wherein detecting the airway microbiome in the biological sample comprises amplifying and sequencing 16S rRNA genes of microorganisms in the sample.

### Embodiment 4

The method of any one of Embodiments 1-3, wherein detecting the airway microbiome in the biological sample comprises amplifying and sequencing the V4 region of 16S rRNA genes of microorganisms in the sample.

### Embodiment 5

The method of any one of Embodiments 1-4, wherein detecting the airway microbiome in the biological sample comprises determining the number of families, the number of genera, the number of species, the Faith's Phylogenetic Diversity, the Shannon Diversity, and/or the Simpson Diversity of the microorganisms.

### Embodiment 6

The method of any one of Embodiments 1-5, wherein the airway microbiome is a lung microbiome.

### Embodiment 7

The method of any one of Embodiments 1-6, wherein detecting the airway microbiome in the biological sample comprises detecting whether
(a) the biological sample has an increased proportion of bacteria in the Pseudomonadaceae family of bacteria compared to a general population of subjects who have pneumonia and are infected with HIV;
(b) the biological sample has an increased proportion of bacteria in the Pseudomonadaceae family of bacteria compared to a general population of subjects who have pneumonia and are infected with HIV, wherein the increased proportion is a proportion of at least about 10%, 20%, 30%, 40%, or 50%;
(c) at least 40% of the bacteria in the biological sample are in the Pseudomonadaceae family of bacteria, at least 10% of the bacteria in the biological sample are in the Sphingomonadaceae family of bacteria, and at least 5% of the bacteria in the biological sample are in the Prevotellaceae family of bacteria;
(d) the biological sample has an increased proportion of bacteria in the Streptococcaceae family of bacteria compared to a general population of subjects who have pneumonia and are infected with HIV;
(e) the biological sample has an increased proportion of bacteria in the Streptococcaceae family of bacteria compared to a general population of subjects who have pneumonia and are infected with HIV, wherein the increased proportion is a proportion of at least about 10%, 20%, 30%, 40%, or 50%;
(f) at least 40% of the bacteria in the biological sample are in the Streptococcaceae family of bacteria, at least 5% of the bacteria in the biological sample are in the Veillonellaceae family of bacteria, and at least 10% of the bacteria in the biological sample are in the Prevotellaceae family of bacteria;
(g) the biological sample has an increased proportion of bacteria in the Prevotellaceae family of bacteria compared to a general population of subjects who have pneumonia and are infected with HIV;
(h) the biological sample has an increased proportion of bacteria in the Prevotellaceae family of bacteria compared to a general population of subjects who have pneumonia and are infected with HIV, wherein the increased proportion is a proportion of at least about 10%, 20%, 30%, 40%, or 50%; and/or
(i) at least 30% of the bacteria in the biological sample are in the Prevotellaceae family of bacteria, at least 10% of the bacteria in the biological sample are in the Streptococcaceae family of bacteria, and at least 10% of the bacteria in the biological sample are in the Veillonellaceae family of bacteria.

### Embodiment 8

The method of any one of Embodiments 1-7, wherein detecting the airway microbiome in the biological sample comprises detecting whether the subject
(a) has an increased proportion of lung microbiome bacteria in the Pseudomonadaceae family of bacteria compared to a healthy or general population of subjects;
(b) has an increased proportion of lung microbiome bacteria in the Pseudomonadaceae family of bacteria compared to a healthy or general population of subjects, wherein the increased proportion is a proportion of at least about 10%, 20%, 30%, 40%, or 50%; and/or
(c) has a proportion of at least 40% of lung microbiome bacteria in the Pseudomonadaceae family of bacteria, at least 10% of lung microbiome bacteria in the Sphingomonadaceae family of bacteria, and at least 5% of lung microbiome bacteria in the Prevotellaceae family of bacteria.

### Embodiment 9

The method of any one of Embodiments 1-7, wherein detecting the airway microbiome in the biological sample comprises detecting whether the subject
(a) has an increased proportion of lung microbiome bacteria in the Pseudomonadaceae family of bacteria compared to a healthy or general population of subjects;
(b) has an increased proportion of lung microbiome bacteria in the Pseudomonadaceae family of bacteria compared to a healthy or general population of subjects, wherein the increased proportion is a proportion of at least about 10%, 20%, 30%, 40%, or 50%; and/or
(c) has a proportion of at least 40% of lung microbiome bacteria in the Pseudomonadaceae family of bacteria, at least 10% of lung microbiome bacteria in the Sphingomonadaceae family of bacteria, and at least 5% of lung microbiome bacteria in the Prevotellaceae family of bacteria.

### Embodiment 10

The method of any one of Embodiments 1-7, wherein detecting the airway microbiome in the biological sample comprises detecting whether the subject
(a) has an increased proportion of lung microbiome bacteria in the Prevotellaceae family of bacteria compared to a healthy or general population of subjects;
(b) has an increased proportion of lung microbiome bacteria in the Prevotellaceae family of bacteria compared to a healthy or general population of subjects, wherein the increased proportion is a proportion of at least about 10%, 20%, 30%, 40%, or 50%; and/or
(c) has a proportion of at least 30% of lung microbiome bacteria in the Prevotellaceae family of bacteria, at least 10% lung microbiome bacteria in the Streptococcaceae family of bacteria, and at least 10% lung microbiome bacteria in the Veillonellaceae family of bacteria.

### Embodiment 11

The method of any one of Embodiments 1-7, wherein detecting the airway microbiome in the biological sample comprises detecting whether the subject
(a) has an increased proportion of lung microbiome bacteria in the Prevotellaceae family of bacteria compared to the general population;
(b) has an increased proportion of lung microbiome bacteria in the Prevotellaceae family of bacteria compared to the general population, wherein the increased proportion is a proportion of at least about 10%, 20%, 30%, 40%, or 50%; and/or
(c) has a proportion of at least 30% of lung microbiome bacteria in the Prevotellaceae family of bacteria, at least 10% of lung microbiome bacteria in the Streptococcaceae family of bacteria, and at least 10% of lung microbiome bacteria in the Veillonellaceae family of bacteria.

### Embodiment 12

A method of detecting at least one immune protein or immune protein-encoding mRNA in a subject who has or is suspected of having a lung infection, the method comprising detecting the level of TIM-3 protein or TIM-3 mRNA in a biological sample from the subject.

### Embodiment 13

The method of Embodiments 12, comprising determining whether the level TIM-3 protein or TIM-3 mRNA is increased compared to a general or healthy population of subjects.

### Embodiment 14

The method of Embodiments 12 or 13, further comprising detecting the level of any one of or any combination of 2, 3, 4, 5, 6, 7, 8, 9, 10, 11, 12, 13, 14, 15, 16, 17, 18, or 19 of

(i) IFNγ protein, IFNα protein, TNFα protein, MUC5AC protein, IL-17A protein, IL-4 protein, IL-5 protein, IL-13 protein, IL-33 protein, OCEL1 protein, CCL11 protein, PADI4 protein, IL-10 protein, FOXP3 protein, PD-1 protein, CD45RO protein, CD2 protein, CD39 protein, and GAPDH protein; or

(ii) IFNγ mRNA, IFNα mRNA, TNFα mRNA, MUC5AC mRNA, IL-17A mRNA, IL-4 mRNA, IL-5 mRNA, IL-13 mRNA, IL-33 mRNA, OCEL1 mRNA, CCL11 mRNA, PADI4 mRNA, IL-10 mRNA, FOXP3 mRNA, PD-1 mRNA, CD45RO mRNA, CD2 mRNA, CD39 mRNA, and GAPDH mRNA,

in the biological sample.

### Embodiment 15

The method of any one of Embodiments 12-14, wherein no more than 1000, 900, 800, 700, 600, 500, 400, 300, 200, 100, 50, 20, or 10 proteins or mRNAs are detected.

### Embodiment 16

The method of any one of Embodiments 12-15, wherein the detecting does not comprise the use of a microarray.

### Embodiment 17

The method of any one of Embodiments 12-15, wherein the detecting comprises the use of a microarray that detects the mRNA levels of less than 1000, 900, 800, 700, 600, 500, 400, 300, 200, 100, 50, 20, or 10 genes.

### Embodiment 18

The method of any one of Embodiments 12-17, comprising detecting whether the subject has an increased level of expression of 1 of or any combination of 2, 3, 4, 5, 6, 7, 8, 9, 10, 11, 12, 13, 14, 15, or 16 of IFNγ, IFNα, TNFα, MUC5AC, IL-17A, IL-4, IL-5, IL-13, IL-33, OCEL1, CCL11, FOXP3, PD-1, CD45RO, CD39, and/or GAPDH compared to a general or healthy population.

### Embodiment 19

The method of any one of Embodiments 12-17, comprising detecting whether the subject has an increased level of expression of 1 of or any combination of 2, 3, 4, 5, 6, 7, 8, 9, 10, 11, 12, 13, 14, 15, or 16 of IFNγ, IFNα, TNFα, MUC5AC, IL-17A, IL-4, IL-5, IL-13, IL-33, OCEL1, CCL11, FOXP3, PD-1, CD45RO, CD39, and/or GAPDH compared to the general population.

### Embodiment 20

The method of any one of Embodiments 1-19, wherein the biological sample is from an airway of the subject.

### Embodiment 21

The method of any one of Embodiments 1-20, wherein the airway is in a lung of the subject.

### Embodiment 22

The method of any one of Embodiments 1-21, wherein the airway is a trachea, bronchus, bronchiole, alveolar duct, alveolar sac, and/or alveolus.

### Embodiment 23

The method of any one of Embodiments 1-22, wherein the biological sample is a bronchoalveolar lavage (BAL) sample, sputum, phlegm, saliva, or mucus.

### Embodiment 24

A method of detecting at least one metabolite in a subject who has or is suspected of having a lung infection, the method comprising detecting the at least one metabolite in a biological sample from the subject, wherein the at least one metabolite is a tryptophan metabolite, an arachidonic acid metabolite, an eicosanoid, or a product of primary or secondary bile metabolism.

### Embodiment 25

The method of Embodiments 24, wherein the biological sample is a bodily fluid.

### Embodiment 26

The method of Embodiment 25, wherein the bodily fluid is blood, serum, or plasma.

### Embodiment 27

The method of any one of Embodiments 24-26, wherein the at least one metabolite is leukotriene B4, xanthurenate, 15-hydroxyeicosatetraenoic acid, chenodeoxycholate, glycodeoxycholate, taurochenodeoxycholate, or ursodeoxycholate.

### Embodiment 28

The method of any one of Embodiments 24-26, wherein the at least one metabolite is a lysolipid metabolite, a pyrimidine metabolite, or a monoacylglycerol.

### Embodiment 29

The method of any one of Embodiments 24-26, wherein the at least one metabolite is a valine metabolite, a leucine metabolite, or a monoacylglycerol associated with lipid metabolism.

### Embodiment 30

The method of any one of Embodiments 24-26, wherein the at least one metabolite is 3-methyl-2-oxobutyrate, 4-methyl-2-oxopentanoate, 1-dihomo-linolenylglycerol, 1-myristoylglycerol, or inosine.

### Embodiment 31

The method of any one of Embodiments 24-26, comprising detecting whether the subject has an increased level of 1, 2, 3, 4, 5, 6, or 7 of leukotriene B4, xanthurenate, 15-hydroxyeicosatetraenoic acid, chenodeoxycholate, glycodeoxycholate, taurochenodeoxycholate, and/or ursodeoxycholate compared to a general or healthy population of subjects.

### Embodiment 32

The method of any one of Embodiments 24-26, comprising detecting whether the subject has an increased level of 1, 2, 3, 4, 5, 6, or 7 of leukotriene B4, xanthurenate, 15-hydroxyeicosatetraenoic acid, chenodeoxycholate, glycodeoxycholate, taurochenodeoxycholate, and/or ursodeoxycholate compared to a general or healthy population of subjects, or has an increased level of 1, 2, 3, or 5 or 3-methyl-2-oxobutyrate, 4-methyl-2-oxopentanoate, 1-dihomo-linolenylglycerol, 1-myristoylglycerol, or inosine compared to a general or healthy population of subjects.

### Embodiment 33

The method of any one of Embodiments 24-26, comprising detecting whether the subject has an increased level of 1, 2, 3, or 5 or 3-methyl-2-oxobutyrate, 4-methyl-2-oxopentanoate, 1-dihomo-linolenylglycerol, 1-myristoylglycerol, or inosine compared to the general population.

### Embodiment 34

The method of any one of Embodiments 1-33, wherein the subject is infected with human immunodeficiency virus (HIV) or is suspected of being infected with HIV.

### Embodiment 35

The method of any one of Embodiments 1-34, wherein the subject has or is suspected of having pneumonia.

### Embodiment 36

The method of Embodiment 35, wherein the pneumonia is bacterial pneumonia or fungal pneumonia.

### Embodiment 37

The method of any one of Embodiments 1-36, wherein the subject has or is suspected of having tuberculosis (TB).

### Embodiment 38

The method of any one of Embodiments 1-37, wherein the subject has or is suspected of having TB pneumonia.

### Embodiment 39

The method of any one of Embodiments 1-38, wherein the subject has been administered an antibiotic.

### Embodiment 40

The method of Embodiment 39, wherein the antibiotic is ceftriaxone.

### Embodiment 41

A method of treating or preventing a Mycobacterium sp. infection in a subject in need thereof, the method comprising administering an effective amount of at least one antibiotic compound to the subject, wherein the subject
(a) has increased TIM-3 expression compared to a general or healthy population of subjects;
(b) has an increased level of 1, 2, 3, 4, 5, 6, or 7 of leukotriene B4, xanthurenate, 15-hydroxyeicosatetraenoic acid, chenodeoxycholate, glycodeoxycholate, taurochenodeoxycholate, and/or ursodeoxycholate compared to a general or healthy population of subjects;
(c) has an increased proportion of lung microbiome bacteria in the Pseudomonadaceae family of bacteria compared to a healthy or general population of subjects;
(d) has an increased proportion of lung microbiome bacteria in the Pseudomonadaceae family of bacteria compared to a healthy or general population of subjects, wherein the increased proportion is a proportion of at least about 10%, 20%, 30%, 40%, or 50%; and/or
(e) has a proportion of at least 40% of lung microbiome bacteria in the Pseudomonadaceae family of bacteria, at least 10% of lung microbiome bacteria in the Sphingomonadaceae family of bacteria, and at least 5% of lung microbiome bacteria in the Prevotellaceae family of bacteria.

### Embodiment 42

A method of treating or preventing a Mycobacterium sp. infection in a subject in need thereof, the method comprising:
(a) detecting (i) an airway microbiome; (ii) a diversity of microorganisms; (iii) a plurality of microorganisms; and/or (iv) at least one metabolite, in a biological sample from the subject; and
(b) administering to the subject an effective amount of at least one antibiotic compound.

### Embodiment 43

The method of Embodiment 41, wherein the at least one antibiotic compound is 1, 2, 3, or 4 of any combination of isoniazid, rifampin, ethambutol, and/or pyrazinamide.

### Embodiment 44

The method of Embodiment 41, wherein the at least one antibiotic compound is isoniazid, rifampin, and pyrazinamide.

### Embodiment 45

The method of Embodiment 41, wherein the at least one antibiotic compound is isoniazid and rifampin.

### Embodiment 46

The method of Embodiment 41, wherein the at least one antibiotic compound is an aminoglycoside, a fluoroquinolone, a polypeptide, a thioamide, a cycloserine, and/or p-aminosalicylic acid.

### Embodiment 47

A method of treating or preventing an Aspergillus sp. infection in a subject in need thereof, the method comprising administering an effective amount of at least one antifungal agent to the subject, wherein the subject
(a) has an increased level of expression of 1 of or any combination of 2, 3, 4, 5, 6, 7, 8, 9, 10, 11, 12, 13, 14, 15, or 16 of IFNγ, IFNα, TNFα, MUC5AC, IL-17A, IL-4, IL-5, IL-13, IL-33, OCEL1, CCL11, FOXP3, PD-1, CD45RO, CD39, and/or GAPDH compared to a general or healthy population;
(b) has an increased level of 1, 2, 3, or 5 or 3-methyl-2-oxobutyrate, 4-methyl-2-oxopentanoate, 1-dihomo-linolenylglycerol, 1-myristoylglycerol, or inosine compared to a general or healthy population of subjects;
(c) has an increased proportion of lung microbiome bacteria in the Prevotellaceae family of bacteria compared to a healthy or general population of subjects;
(d) has an increased proportion of lung microbiome bacteria in the Prevotellaceae family of bacteria compared to a healthy or general population of subjects, wherein the increased proportion is a proportion of at least about 10%, 20%, 30%, 40%, or 50%; and/or
(e) has a proportion of at least 30% of lung microbiome bacteria in the Prevotellaceae family of bacteria, at least 10% lung microbiome bacteria in the Streptococcaceae family of bacteria, and at least 10% lung microbiome bacteria in the Veillonellaceae family of bacteria.

### Embodiment 48

A method of treating or preventing an Aspergillus sp. infection in a subject in need thereof, the method comprising:
(a) detecting (i) an airway microbiome; (ii) the diversity of microorganisms; (iii) a plurality of microorganisms; and/or (iv) at least one metabolite, in a biological sample from the subject; and
(b) administering to the subject an effective amount of at least one antifungal agent.

### Embodiment 49

The method of Embodiment 50, wherein the at least one antifungal agent is a triazole antifungal agent.

### Embodiment 50

The method of Embodiment 50, wherein the at least one antifungal agent is 1, 2, 3, 4, 5, 6, or 7 of any combination of amphotericin B, liposomal amphotericin B, voriconazole, caspofungin, flucytosine, itraconazole, and/or posaconazole.

### Embodiment 51

The method of any one of Embodiments 41-46, wherein the Mycobacterium sp. is M. tuberculosis.

### Embodiment 52

The method of any one of Embodiments 47-50, wherein the Aspergillus sp. is A. fumigatus or A. flavus.

### Embodiment 53

A method of treating or preventing a lung infection in a subject in need thereof, the method comprising administering an effective amount of at least one antibiotic or antifungal agent to the subject, wherein the subject
(a) has an increased level of expression of 1 of or any combination of 2, 3, 4, 5, 6, 7, 8, 9, 10, 11, 12, 13, 14, 15, or 16 of IFNγ, IFNα, TNFα, MUC5AC, IL-17A, IL-4, IL-5, IL-13, IL-33, OCEL1, CCL11, FOXP3, PD-1, CD45RO, CD39, and/or GAPDH compared to a general or healthy population;
(b) has an increased level of 1, 2, 3, or 5 or 3-methyl-2-oxobutyrate, 4-methyl-2-oxopentanoate, 1-dihomo-linolenylglycerol, 1-myristoylglycerol, or inosine compared to a general or healthy population of subjects;
(c) has an increased proportion of lung microbiome bacteria in the Prevotellaceae family of bacteria compared to a healthy or general population of subjects;
(d) has an increased proportion of lung microbiome bacteria in the Prevotellaceae family of bacteria compared to a healthy or general population of subjects, wherein the increased proportion is a proportion of at least about 10%, 20%, 30%, 40%, or 50%;
(e) has a proportion of at least 30% of lung microbiome bacteria in the Prevotellaceae family of bacteria, at least 10% lung microbiome bacteria in the Streptococcaceae family of bacteria, and at least 10% lung microbiome bacteria in the Veillonellaceae family of bacteria;
(f) has increased TIM-3 expression compared to a general or healthy population of subjects;
(g) has an increased level of 1, 2, 3, 4, 5, 6, or 7 of leukotriene B4, xanthurenate, 15-hydroxyeicosatetraenoic acid, chenodeoxycholate, glycodeoxycholate, taurochenodeoxycholate, and/or ursodeoxycholate compared to a general or healthy population of subjects;
(h) has an increased proportion of lung microbiome bacteria in the Pseudomonadaceae family of bacteria compared to a healthy or general population of subjects;
(i) has an increased proportion of lung microbiome bacteria in the Pseudomonadaceae family of bacteria compared to a healthy or general population of subjects, wherein the increased proportion is a proportion of at least about 10%, 20%, 30%, 40%, or 50%; and/or
(j) has a proportion of at least 40% of lung microbiome bacteria in the Pseudomonadaceae family of bacteria, at least 10% of lung microbiome bacteria in the Sphingomonadaceae family of bacteria, and at least 5% of lung microbiome bacteria in the Prevotellaceae family of bacteria.

### Embodiment 54

A method of treating or preventing a lung infection in a subject in need thereof, the method comprising:
(a) detecting (i) an airway microbiome; (ii) the diversity of microorganisms; (iii) a plurality of microorganisms; and/or (iv) at least one metabolite, in a biological sample from the subject; and
(b) administering to the subject an effective amount of at least one antibiotic or antifungal agent.

### Embodiment 55

The method of any one of Embodiments 41-54, wherein the subject is infected with human immunodeficiency virus (HIV) or is suspected of being infected with HIV.

### Embodiment 56

The method of any one of Embodiments 41-55, wherein the subject has pneumonia.

### Embodiment 57

The method of Embodiment 56, wherein the pneumonia is bacterial pneumonia.

### Embodiment 58

The method of any one of Embodiments 41-57, wherein the subject has tuberculosis (TB).

### Embodiment 59

The method of any one of Embodiments 41-58, wherein the subject has TB pneumonia.

### Embodiment 60

The method of any one of Embodiments 41-59, wherein the subject has been administered an antibiotic.

### Embodiment 61

The method of Embodiment 60, wherein the antibiotic is ceftriaxone.

### EXAMPLES

The following examples are offered to illustrate, but not to limit the claimed invention.

### Example 1. Immune Response and Mortality Risk Relate to Distinct Lung Microbiomes in Hiv-Pneumonia Patients

HIV-infected pneumonia patients exhibit greater lung microbial diversity than non-HIV pneumonia patients; however, it is unknown if distinct pathogenic lower airway microbiomes exist in HIV-infected pneumonia patients and whether these relate to host immune response and mortality within this population.

Using a cohort of 182 HIV-infected pneumonia patients, three compositionally distinct microbial community states in the lower airways were identified. Each exhibits unique metagenomic functional capacity, induces distinct lower airway immune responses, and is associated with a unique profile of circulating metabolites and with different rates of mortality. Without being bound by any scientific theory, these results provide evidence that microbiological and immunologically distinct subsets of HIV-infected pneumonia patients exist and that these distinctions are related to clinical outcomes, thus arguing for the potential need to tailor therapy based on the specific microbiome dysbiosis and related immune and metabolic dysfunction exhibited by these patients.

The potential role of the airway microbiota in dictating immune responses and infection outcomes in HIV-associated pneumonia is largely unknown. The objective was to investigate whether microbiologically and immunologically distinct subsets of HIV-infected pneumonia patients exist and are related to mortality. Ugandan HIV-infected pneumonia patient (n=182) bronchoalveolar lavage (BAL) samples were obtained at study enrollment (following antibiotic treatment); patient demographics, including 8 and 70 day mortality were collected. Lower airway bacterial community composition was assessed via amplification and sequencing of the V4 region of the 16S rRNA gene. Host immune response gene expression profiles were generated using RNA extracted from bronchoalveolar lavage fluid using QPCR. Liquid and gas chromatography mass spectrometry was used to profile serum metabolites.

Without being bound by any scientific theory, based on airway microbiome composition, the majority of patients segregated into three distinct groups, each of which were predicted to encode metagenomes capable of producing metabolites characteristically enriched in paired serum samples from these patients. These three groups also exhibited differences in mortality; those with the highest rate had increased ceftriaxone administration and culturable Aspergillus, and demonstrated significantly increased induction of airway T-helper 2 responses. The group with the lowest mortality was characterized by increased expression of T-cell immunoglobulin and mucin domain 3 (TIM-3), which down-regulates T-helper 1 pro-inflammatory responses and is associated with chronic viral infection.

Without being bound by any scientific theory, these data provide evidence that compositionally and structurally distinct lower airway microbiomes are associated with discrete local host immune responses, peripheral metabolic reprogramming, and different rates of mortality.

Sub-Saharan Africa accounts for 71% of persons estimated to be living with human immunodeficiency virus-(HIV)-infection worldwide, with 63,000 acquired immunodeficiency syndrome-(AIDS)-related deaths per year in Uganda alone (1). Pulmonary infections pose a common and frequently fatal co-morbidity in HIV-infected patients in Africa; two of the most prevalent are tuberculosis (TB) and bacterial pneumonia, incident in approximately 80% of this patient population (2). Overall, TB is the leading cause of death in HIV-infected patients worldwide (3, 4). In HIV-TB co-endemic areas, the most common cause of hospital admission is often bacterial pneumonia, with mortality rates over 30% even with antiretroviral and antibiotic treatments (5, 6).

Even in the absence of acute respiratory infection, HIV-infected patients exhibit a broader breadth of lower airway bacterial taxa compared to that detected in healthy subjects (7, 8), indicating that HIV-infection may present a risk factor for developing pulmonary infection. Despite high morbidity and mortality within this population, little is known about whether variation in airway microbiota composition and immune response are related to patient outcome. It has been demonstrated, in a non-HIV-infected, antimicrobial-treated pneumonia cohort, that following antimicrobial treatment, a precipitous decline in airway microbiome diversity and domination of the community by a distinct respiratory pathogen e.g. Streptococcus pneumonia or Pseudomonas aeruginosa is associated with increased 28-day mortality (9). In a study of 60 Ugandan HIV-infected, antimicrobial-treated pneumonia patients, patients with reduced airway bacterial microbiota richness and diversity exhibited higher bacterial burden and increased expression of pro-inflammatory TNFα and MMP-9 (10), thus providing the first evidence that HIV-airway microbiota composition is related to immune response. Without being bound by any scientific theory, these observations led to the hypothesis that with acute pneumonia, in the context of HIV-associated immune dysfunction and antimicrobial administration, patients subsets can be identified based on their lower airway bacterial composition. It is further rationalized that these compositionally distinct airway microbiota function as discrete pathogenic units which induce characteristic airway immune responses and are associated with mortality. To address this hypothesis, clinical and demographic factors related to the bacterial airway microbiome, as well as relationships between community composition, host immune response, and patient outcomes were examined in a large cohort of Ugandan HIV-infected pneumonia patients. Some of the results of these studies have been previously reported in an abstract (11).

Materials and Methods

Subjects and sample collection. We enrolled HIV-infected subjects admitted to Mulago Hospital in Kampala, Uganda for acute pneumonia from October 2009-December 2011 as part of the Lung Microbiome in Cohorts of HIV-Infected Persons (Lung MicroCHIP) Study. Patients underwent two sputum acid fast bacilli (AFB) smear examinations to diagnose pulmonary TB. AFB smear-negative patients underwent bronchoscopy with BAL for clinical diagnosis, with 10 mL set aside for microbiome analysis (10). Bronchoscopy was performed a median of 3 days after hospital admission (interquartile range 1-4 days). Over 98% of subjects had received antibiotics prior to bronchoscopy. Serum was collected at enrollment. Clinical data was collected and diagnoses were assigned as previously described (10). Study endpoint was mortality follow-up at 70-days post bronchoscopy.

Ethics Statement.

The Makerere University School of Medicine Research Ethics Committee, the Mulago Hospital Research and Ethics Committee, the Uganda National Council for Science and Technology, and the University of California San Francisco Committee on Human Research approved the protocol. Subjects provided written, informed consent.

DNA and RNA Extraction.

Total DNA and RNA were extracted from whole BAL in parallel using an AllPrep DNA/RNA extraction kit [Qiagen, (7)]; RNA quality and purity were assessed as previously described (10).

16S rRNA Gene Amplification and Sequencing.

The V4-region of bacterial 16S rRNA gene was amplified using primers with multiplex sequencing barcodes (Table 1). A mock community was used to monitor for contamination and standardize across runs. Sequencing was performed using a MiSeq platform and MiSeq Control Software v2.2.0 (Illumina).

TABLE 1
Sequencing primers

| Primer Name | Primer Sequence |
|---|---|
| 515F_w_adapt- | 5′-AATGATACGGCGACCACCGAGATCTACAC- |
| er | TATGGTAATT-GTGTGCCAGCMGCCGCGGTAA -3′ |
| 806R_w_adapt- | 5′-CAAGCAGAAGACGGCATACGAGAT- |
| er | XXXXXXXXXXXX-AGTCAGTCAG- |
| | CCGGACTACHVGGGTWTCTAAT-3′^{a} |

^{a}12 “X” represents 12-bp Golay barcode

The sequence of the 515F_w_adapter (5′-AATGATACGGCGACCACCGAGATCTACACTATGGTAATTGTGTGCCAGCMGCCGCG GTAA-3′) is SEQ ID NO: 1.

The sequence of 806R_w_adapter is (5′-CAAGCAGAAGACGGCATACGAGATXXXXXXXXXXXXAGTCAGTCAGCCGGACTAC HVGGGTWTCTAAT-3′) is SEQ ID NO: 2.

Microbiome Data Processing.

251 bp paired-end sequence reads were assembled using FLASh (12), and quality-trimmed using QIIME [(13)]. Chimeras were removed using ChimeraSlayer (14). Each sample was rarefied 100 times to 100,000 reads in the R environment (15); the centroid of each sample distribution was subsequently used for analysis (n=182). Greengenes database May 2013 (16) was used to classify taxa; singleton OTUs were removed.

Immune Gene Expression.

Total RNA (0.5 ug) was reverse transcribed; cDNA gene expression was assayed using Real time PCR and analyzed using the delta-delta CT method to normalize gene expression (17).

Metabolic Profiling.

Metabolic profiles were generated from 100 uL of patient serum (n=30) by Ultrahigh Performance Liquid Chromatography-Tandem Mass Spectrometry (UPLC-MS/MS) and Gas Chromatography-Mass Spectrometry (GC-MS) at Metabolon according to a standard protocol.

Microbial and Statistical Analyses.

Microbial analyses were performed using QIIME software (13). Results were visualized using Emperor (18). Metagenomic predictions were generated using PICRUSt (19). Procrustes (“transform_coordinate_matrices.py” script “-r 1000”) and Mantel tests (“compare_distance_matrices.py” script) were performed in QIIME using Bray Curtis dissimilarity [compositional dissimilarity based on taxon relative abundance (20)]. Statistical analyses, e.g. one-way ANOVA, Kruskal-Wallis, etc. were performed in the R-environment. Dirichlet Multinomial Mixtures and log-rank test were performed using the DirichletMultinomial (21) and survival packages, respectively. Permutational Multivariate Analysis of Variance [PERMANOVA (22), vegan v.2.3.0, 1000 permutations] and Principal Coordinates Analysis (PCoA) were performed using weighted UniFrac (23, 24) and Canberra dissimilarity measurements. PERMANOVA independently considers each factor e.g. age, gender against bacterial community beta-diversity variance, permuting data 1000 times, and thus does not require false discovery correction. The resulting R² provides the proportion of variation explained e.g. a factor that has a R²=0.021, explains 2% of the variation in community composition.

Sample and Clinical Data Collection.

Bronchoscopy with BAL was performed, and BAL was stored 1:2 in RNALater (Thermo Fisher Scientific). Notably, during the bronchoscopy, suction is not started until the bronchoscope has been wedged, limiting oral contamination. Venipuncture for blood specimens was performed at enrollment, hospital day #1, and collected into serum separator tubes. Clinical data were collected using standardized forms. MCS groups were rigorously checked across dates of collection, shipping groups, and sequencing batches to ensure that microbial community was not a result of collection, shipping, or sample processing methods. Each of the three MCS described were observed throughout the timeline of sample collection and processing.

BAL Processing and Extraction.

BAL was stored 1:2 vol vol in RNALater for stable storage and shipping from Uganda to San Francisco. RNA and DNA were extracted in parallel from whole BAL. Thawed BAL samples were centrifuged at high speed, the supernatant was removed and pellets were resuspended in sterile PBS, and centrifuged again at high speed, prior to extraction. Total DNA and RNA were extracted from whole BAL in parallel using an AllPrep DNA/RNA extraction kit (Qiagen) according to manufacturer's instructions.

16S rRNA Gene Amplification and Sequencing.

The V4-region of bacterial 16S rRNA gene was amplified using primers with barcodes for multiplex sequencing (Table 1). PCRs were performed in triplicate with parallel no-template control reactions in which no amplification product was observed. Triplicate PCR product was pooled and visualized on 2% agarose gel. If there were no visible background bands >150 bp, Agencourt AMPure XP System (Beckman Coulter) was used for PCR product purification, otherwise the product was purified using QIAquick Gel Extraction Kit (Qiagen). Quality of purified amplicon was confirmed using a Bioanalyzer and the Agilent DNA 1000 Kit (Agilent Technologies). Purified products were quantified using Qubit dsDNA HS Assay Kit (Life Technologies) and pooled equimolar for multiplex sequencing.

A mock community composed of equal genomic concentration (2 ng per reaction) of Escherichia coli ATCC25922, Pseudomonas aeruginosa ATCC27853, Corynebacterium tuberculostearicum ATCC35692, Lactobacillus sakei ATCC15521, and L. rhamnosus ATCC53103 was used to monitor and standardize data between runs.

Microbiome Data Processing.

Each 251 bp paired-end read was assembled using FLASh (Fast Length Adjustment of SHort reads) with parameters: -r 251 -f 300 -s 30 -m 15. Assembled sequences were quality trimmed using QIIME software with default settings except Phred quality threshold -q 30.

Immune Gene Expression.

Total RNA (0.5 ug) was reverse transcribed using the RT First Strand Kit, and cDNA gene expression was assessed on a custom RT Profiler PCR Array (both Qiagen). Real time PCR and detection was performed on QuantStudio 6 Flex (Thermo Fisher Scientific) by two-step cycling: 95° C. for 10 minutes, then 40 cycles: 15 seconds at 95° C., 1 minute at 60° C. The custom PCR array included IFNγ, IFNα, TNFα, MUC5AC, IL-17A, IL-4, IL-5, IL-13, IL-33, OCEL1, CCL11, PADI4, IL-10, FOXP3, PD-1, TIM-3, CD45RO, CD2, CD39, and GAPDH; the latter was used with the delta-delta CT method to normalize host immune gene expression.

Results

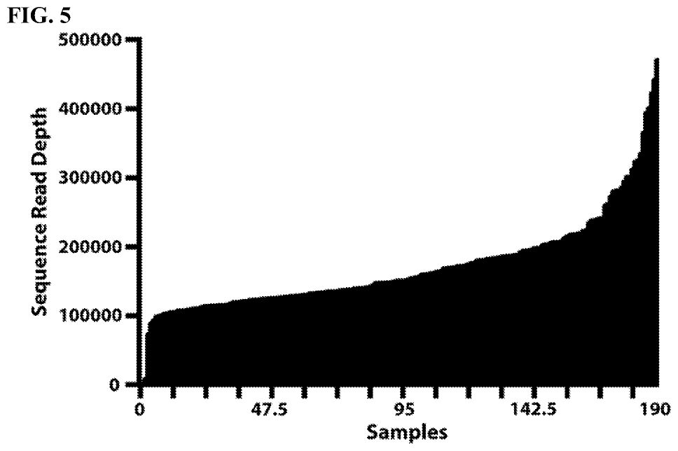

Lower airway microbiota composition is associated with demographic, clinical and microbiological factors. Lower airway bacterial microbiota profiles of 190 Ugandan HIV-infected patients with acute pneumonia were generated by 16S rRNA amplicon sequencing of whole BAL fluid (FIG. 5: read depth). Overall, 182 samples with sufficient sequence reads and adequate bacterial community coverage were used for all microbiota analyses. A total of 6,915 operational taxonomical units (OTUs; >97% 16S rRNA V4-sequence similarity; range 124-869, median 335.5 taxa per sample) were identified indicating robust bacterial presence.

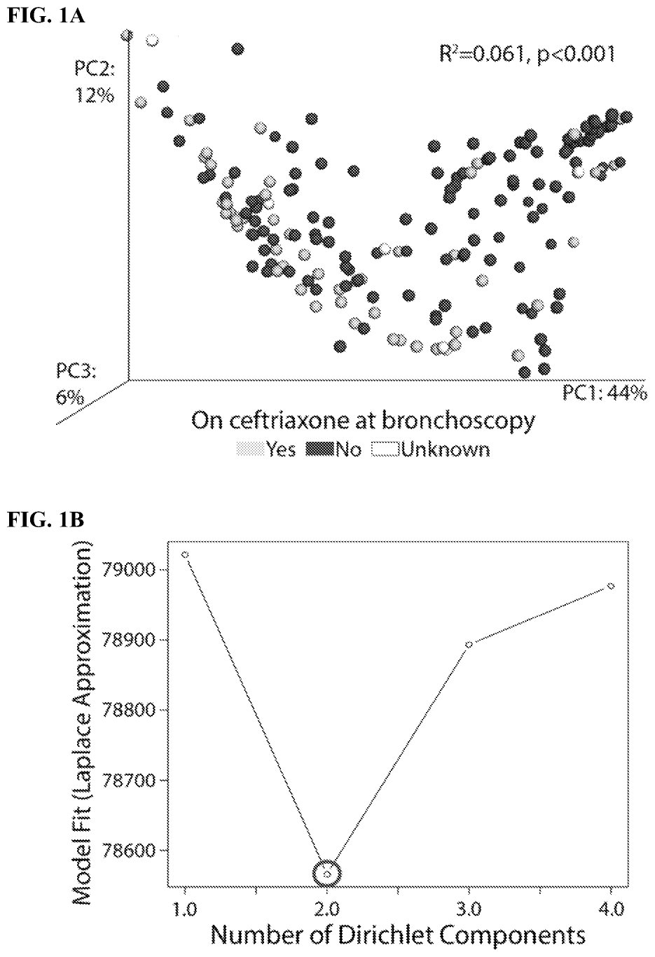

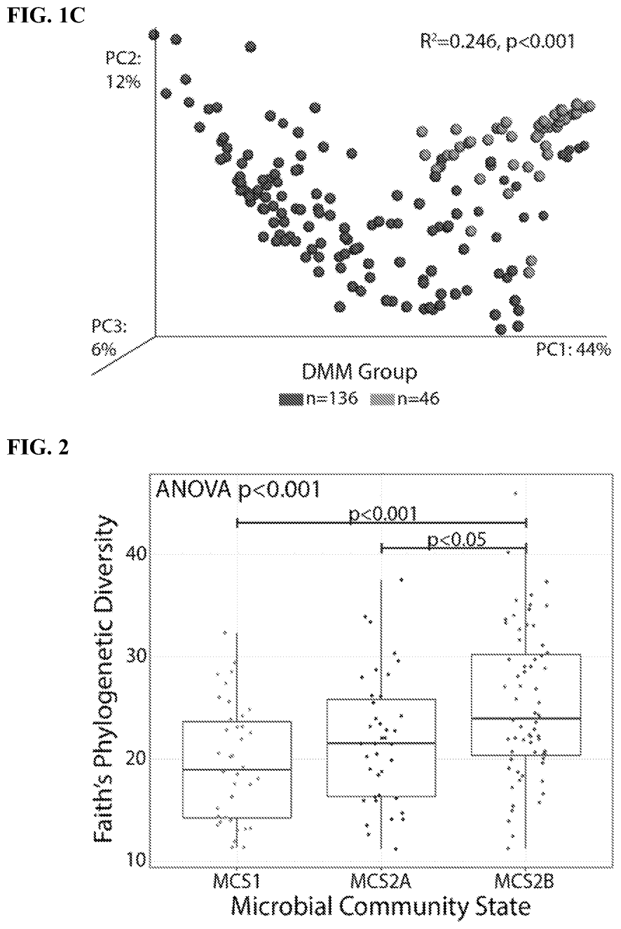

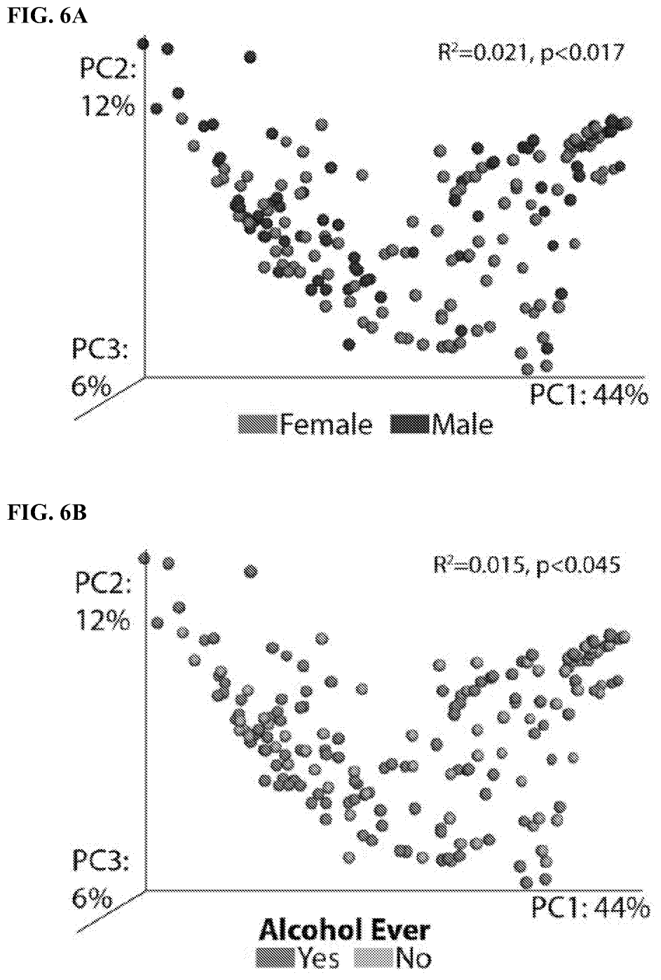

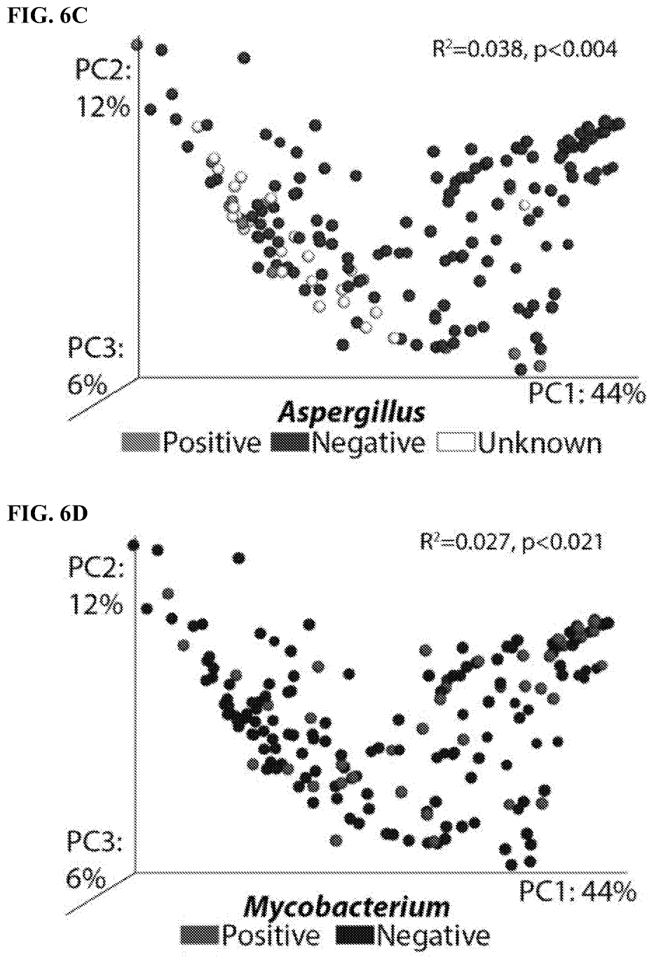

Demographic and clinical data (Table 2) were used in PERMANOVA analysis (22). PERMANOVA allows for the identification of factors related to observed variation in bacterial beta-diversity (inter-sample bacterial composition differences); beta-diversity was measured using a weighted UniFrac dissimilarity matrix, which considers phylogenetic relatedness and species abundance in distance calculations (24). Gender (R²=0.021; p<0.017, FIG. 6A), consumption of alcohol ever (R²=0.015, p<0.045; FIG. 2B), the presence of culturable Aspergillus in BAL (R²=0.038, p<0.004; FIG. 6C), BAL or sputum culture positivity for Mycobacterium (R²=0.027, p<0.021; FIG. 6D), and ceftriaxone administration within the last two weeks, or at the time of bronchoscopy (R²=0.016, p<0.040 and R²=0.061, p<0.001 respectively; Table 3; FIG. 1A) were significantly related to airway bacterial community composition. Seventy-day mortality trended strongly towards a relationship with airway microbiota composition [Canberra (beta-diversity distance based on taxa presence/absence); R²=0.0061, p<0.053]. Mortality trended strongly towards significance using a Canberra but not a weighted UniFrac dissimilarity matrix, suggesting that presence (or absence) of particular taxa in airway communities is related to mortality, rather than relative abundance or phylogenetic relatedness of community members present.

TABLE 2
Summary of demographic variables.

| | | Yes \| No | Statistical | | | | | |
|---|---|---|---|---|---|---|---|---|
| | Sam- | Or | Test | | | | | |
| Variable | ples | Range | across | | | | | |
| Name | (n) | [min-max (med)] | MCS | MCS1 | MCS2A | MCS2B | p-value | Sig. |
| Pneumocystis | 182 | 6 \| 176 (y\|n) | χ² test | 1 \| 37 | 3 \| 36 | 2 \| 65 | 0.43 | |
| positive | | | | | | | | |
| (diagnosis by | | | | | | | | |
| BAL | | | | | | | | |
| microscopy) | | | | | | | | |
| Mycobacteria | 182 | 41 \| 141 (y\|n) | χ² test | 23 \| 15 | 32 \| 7 | 58 \| 9 | 0.006 | ** |
| culture | | | | | | | | |
| positive | | | | | | | | |
| Aspergillus | 157 | 15 \| 142 (y\|n) | χ² test | 1 \| 37 | 4 \| 31 | 9 \| 41 | 0.07 | † |
| culture | | | | | | | | |
| positive | | | | | | | | |
| Pulmonary | 182 | 14 \| 168 (y\|n) | χ² test | 2 \| 36 | 2 \| 37 | 7 \| 60 | 0.49 | |
| Kaposi's | | | | | | | | |
| Sarcoma | | | | | | | | |
| Age | 182 | 19.1-62.6 (34.1) | One-way | 36.5 | 35.1 | 35.6 | 0.84 | |
| | | | ANOVA | | | | | |
| Gender | 182 | 110 \| 72 (F\|M) | χ² test | 23 \| 15 | 28 \| 11 | 36 \| 31 | 0.19 | |
| Clinical score | 133 | 1 \| 4 \| 59 \| 39 \| 28 \| 2 | χ² test | 0 \| 1 \| 11 \| 4 \| 5 \| 0 | 0 \| 2 \| 16 \| 5 \| 6 \| 0 | 1 \| 1 \| 25 \| 14 \| 8 \| 2 | 0.83 | |
| at admission | | (Normal \| Unaffected \| | | | | | | |
| | | Ambulatory \| <50% in | | | | | | |
| | | bed \| >50% in bed \| Bed | | | | | | |
| | | bound | | | | | | |
| Temperature | 182 | 33.7-39.9 (36.7) | One-way | 36.7 | 37.1 | 36.7 | 0.25 | |
| at admission | | | ANOVA | | | | | |
| (Celsius) | | | | | | | | |
| Heart rate at | 182 | 48-165 (101.5) | One-way | 101.1 | 107.6 | 97.2 | 0.06 | † |
| admission | | | ANOVA | | | | | |
| Respiratory | 182 | 18-64 (30) | One-way | 30.5 | 32.9 | 30.1 | 0.19 | |
| rate at | | | ANOVA | | | | | |
| admission | | | | | | | | |
| (breaths/ | | | | | | | | |
| minute) | | | | | | | | |
| Oxygen | 182 | 76-99 (96) | One-way | 94.3 | 94.6 | 94.7 | 0.90 | |
| saturation at | | | ANOVA | | | | | |
| admission | | | | | | | | |
| Reported | 182 | 159 \| 23 (30) | χ² test | 31 \| 7 | 38 \| 1 | 58 \| 9 | 0.08 | † |
| fevers, chills, | | | | | | | | |
| night sweats | | | | | | | | |
| How long | 159 | 1-24 (4) | One-way | 5.0 | 4.4 | 5.0 | 0.8 | |
| fevers (weeks) | | | ANOVA | | | | | |
| Weight loss at | 182 | 170 \| 12 (y\|n) | χ² test | 36 \| 2 | 39 \| 0 | 58 \| 9 | 0.03 | * |
| admission | | | | | | | | |
| Amount of | 170 | 38 \| 132 | χ² test | 11 \| 25 | 9 \| 30 | 12 \| 46 | 0.55 | |
| weight loss | | (<5 kg \| >=5 kg) | | | | | | |
| How long | 182 | 1-24 (4) | One-way | 6.2 | 7.0 | 6.4 | 0.76 | |
| cough (weeks) | | | ANOVA | | | | | |
| Coughing | 182 | 178 \| 4 (y\|n) | χ² test | 38 \| 0 | 39 \| 0 | 64 \| 3 | 0.17 | |
| sputum at | | | | | | | | |
| admission | | | | | | | | |
| How long | 178 | 1-24 (3) | One-way | 4.3 | 5.6 | 4.5 | 0.33 | |
| coughing | | | ANOVA | | | | | |
| sputum | | | | | | | | |
| (weeks) | | | | | | | | |
| Color of | 178 | 93 \| 85 | χ² test | 20 \| 18 | 19 \| 20 | 34 \| 30 | 0.90 | |
| sputum at | | (White or clear \| | | | | | | |
| admission | | Discolored) | | | | | | |
| Blood in | 178 | 35 \| 143 (y\|n) | χ² test | 7 \| 31 | 10 \| 29 | 13 \| 51 | 0.72 | |
| sputum at | | | | | | | | |
| admission | | | | | | | | |
| Dyspnea at | 182 | 82 \| 100 (y\|n) | χ² test | 22 \| 16 | 19 \| 20 | 29 \| 38 | 0.35 | |
| admission | | | | | | | | |
| How long | 82 | 1-24 (4) | One-way | 4.7 | 4.7 | 4.2 | 0.9 | |
| dyspnea | | | ANOVA | | | | | |
| (weeks) | | | | | | | | |
| Severity of | 82 | 42 \| 40 | χ² test | 10 \| 12 | 10 \| 9 | 15 \| 14 | 0.87 | |
| dyspnea | | (Only exercise \| At | | | | | | |
| | | rest) | | | | | | |
| Chest pain at | 182 | 116 \| 66 (y\|n) | χ² test | 24 \| 14 | 28 \| 11 | 42 \| 25 | 0.61 | |
| admission | | | | | | | | |
| How long | 116 | 1-24 (2.5) | One-way | 4.1 | 3.7 | 5.1 | 0.39 | |
| chest pain | | | ANOVA | | | | | |
| (weeks) | | | | | | | | |
| Wheeze at | 182 | 39 \| 143 (y\|n) | χ² test | 10 \| 28 | 10 \| 29 | 13 \| 54 | 0.64 | |
| admission | | | | | | | | |
| How long | 39 | 1-16 (3) | One-way | 3.7 | 4.9 | 3.7 | 0.67 | |
| wheeze | | | ANOVA | | | | | |
| (weeks) | | | | | | | | |
| On | 121 | 94 \| 27 (y\|n) | χ² test | 18 \| 9 | 22 \| 3 | 30 \| 10 | 0.19 | |
| Pneumocystis | | | | | | | | |
| prophylaxis | | | | | | | | |
| On | 121 | 39 \| 82 (y\|n) | χ² test | 8 \| 19 | 8 \| 17 | 13 \| 27 | 0.97 | |
| antiretrovirals | | | | | | | | |
| CD4 count | 179 | 1-697 (71) | One-way | 179 | 119 | 134 | 0.19 | |
| | | | ANOVA | | | | | |
| Previous | 182 | 14 \| 168 (y\|n) | χ² test | 1 \| 37 | 5 \| 34 | 4 \| 63 | 0.19 | |
| diagnosis TB | | | | | | | | |
| Smoked >99 | 182 | 41 \| 141 (y\|n) | χ² test | 10 \| 28 | 4 \| 35 | 19 \| 48 | 0.09 | † |
| cigarettes in | | | | | | | | |
| lifetime | | | | | | | | |
| Alcohol ever | 182 | 113 \| 69 (y\|n) | χ² test | 20 \| 18 | 27 \| 12 | 44 \| 23 | 0.27 | |
| Last known | 182 | 143 \| 39 (live\|dead) | χ² test | 32 \| 6 | 32 \| 7 | 51 \| 16 | 0.56 | |
| vital status | | | | | | | | |
| Vital status 30 | 182 | 163 \| 19 (live\|dead) | χ² test | 35 \| 3 | 35 \| 4 | 58 \| 9 | 0.67 | |
| days post | | | | | | | | |
| bronchoscopy | | | | | | | | |
| Vital status 70 | 182 | 148 \| 34 (live\|dead) | χ² test | 33 \| 5 | 33 \| 6 | 52 \| 15 | 0.44 | |
| days post | | | | | | | | |
| bronchoscopy | | | | | | | | |
| Reported | 178 | 137 \| 41 (y\|n) | χ² test | 30 \| 7 | 30 \| 6 | 48 \| 19 | 0.32 | |
| septrin within | | | | | | | | |
| last 2 weeks | | | | | | | | |
| Reported | 178 | 83 \| 95 (y\|n) | χ² test | 17 \| 20 | 16 \| 20 | 31 \| 36 | 0.98 | |
| penicillin | | | | | | | | |
| within last 2 | | | | | | | | |
| weeks | | | | | | | | |
| Reported | 178 | 137 \| 41 (y\|n) | χ² test | 32 \| 6 | 25 \| 11 | 54 \| 13 | 0.26 | |
| ceftriaxone | | | | | | | | |
| within last 2 | | | | | | | | |
| weeks | | | | | | | | |
| Reported | 172 | 13 \| 159 (y\|n) | χ² test | 3 \| 34 | 3 \| 30 | 7 \| 59 | 0.91 | |
| quinolone | | | | | | | | |
| within last 2 | | | | | | | | |
| weeks | | | | | | | | |
| Reported | 178 | 50 \| 128 (y\|n) | χ² test | 12 \| 25 | 11 \| 25 | 16 \| 51 | 0.59 | |
| macrolide | | | | | | | | |
| within last 2 | | | | | | | | |
| weeks | | | | | | | | |
| Septrin at | 177 | 103 \| 74 (y\|n) | χ² test | 19 \| 18 | 25 \| 11 | 33 \| 34 | 0.13 | |
| bronchoscopy | | | | | | | | |
| Penicillin at | 177 | 46 \| 131 (y\|n) | χ² test | 8 \| 29 | 9 \| 27 | 18 \| 49 | 0.84 | |
| bronchoscopy | | | | | | | | |
| Ceftriaxone at | 174 | 54 \| 120 (y\|n) | χ² test | 1 \| 35 | 11 \| 25 | 29 \| 36 | 6.3 × | *** |
| bronchoscopy | | | | | | | 10⁻⁵ | |
| Quinolone at | 170 | 7 \| 163 (y\|n) | χ² test | 3 \| 34 | 2 \| 31 | 2 \| 63 | 0.53 | |
| bronchoscopy | | | | | | | | |
| Macrolide at | 174 | 27 \| 147 (30) | χ² test | 4 \| 33 | 5 \| 31 | 11 \| 54 | 0.70 | |
| bronchoscopy | | | | | | | | |

Significance:
†p < 0.1,
*p < 0.05,
**p < 0.01,
***p < 0.001

TABLE 3
Clinical, demographic, and microbiological features are significantly associated
with airway bacterial beta-diversity in HIV-infected patients with pneumonia.

| | | Sample | | PERMANOVA | |
|---|---|---|---|---|---|
| Variable Type | Variable Name | (n) | Yes \| No | R² | p-value |
| Clinical and | 70-Day | 182 | 143 \| 39 | 0.006 | 0.053 |
| demographic | Mortality^{a} | | (Live Dead) | | |
| | Alcohol ever | 182 | 113 \| 69 | 0.015 | 0.045 |
| | consumed | | | | |
| | Ceftriaxone at | 174 | 54 \| 120 | 0.061 | 0.001 |
| | bronchoscopy | | | | |
| | Ceftriaxone | 178 | 137 \| 41 | 0.016 | 0.040 |
| | within last 2 | | | | |
| | weeks | | | | |
| | Culture identified | 157 | 15 \| 142 | 0.038 | 0.004 |
| | Aspergillus | | | | |
| | Gender | 182 | 110 \| 72 | 0.021 | 0.017 |
| | | | (F \| M) | | |
| | TB positive by | 182 | 40 \| 1 \| 141 | 0.027 | 0.021 |
| | culture | | (Positive \| Scanty | | |
| | | | Negative) | | |

| | | | Range | | |
|---|---|---|---|---|---|
| | | Sample | [min-max | PERMANOVA | |
| Variable Type | Variable Name | (n) | (med)] | R² | p-value |
| Microbiological | Chao1 | 182 | 170-1326 | 0.080 | 0.001 |
| | | | (484.1) | | |
| | Faith's | 182 | 8.792-45.83 | 0.091 | 0.001 |
| | Phylogenetic | | (21.97) | | |
| | Diversity | | | | |
| | Observed species | 182 | 39-865 | 0.076 | 0.001 |
| | | | (340.5) | | |
| | Shannon diversity | 182 | 0.642-6.427 | 0.169 | 0.001 |
| | | | (4.011) | | |
| | Simpson diversity | 182 | 0.112-0.977 | 0.154 | 0.001 |
| | | | (0.867) | | |

^{a}PERMANOVA value calculated using a Canberra distance matrix.

Without being bound by any scientific theory, since microbes engage in inter-species cell-cell communication that dictates abundance and behavior of other microorganisms in their environment (25, 26), it was rationalized that inter-species interactions also occur in complex multi-species bacterial microbiota, resulting in deterministic community structures. Indeed, Shannon's diversity index (which considers abundance and richness, PERMANOVA: R²=0.17, p<0.001), Faith's Phylogenetic diversity (phylogenetic variation, R²=0.09, p<0.001), Chaol index (species richness estimator, R²=0.08, p<0.001), and observed species richness (total species, R²=0.08, p<0.001), were all significantly associated with airway bacterial beta-diversity. These alpha-diversity indices (measurements of variation within samples) explained a greater degree of microbial community variability (8-17%) than clinical or demographic features (reflected in the strength of PCoA groupings in FIG. 1A), suggesting that microbiological influences appear to play a larger role in defining airway taxonomic content than clinical-demographic features.

HIV Pneumonia Patients Stratify into Two Groups Based on Bacterial Community Composition.

Dirichlet Multinomial Mixtures [DMM (21)] model examines taxa frequencies and determines how many “meta-communities” or microbial community states (MCS), exist within a dataset. Application of DMM to our cohort identified two significantly distinct MCS (n=46 and n=136, R²=0.246, p<0.001) using a Laplace Approximation (FIGS. 1B and 1C) which evaluates model fit [lowest Laplace value corresponds to the number of meta-communities that best-fit the model (FIG. 1B)]. Specific co-occurring bacterial families were characteristically enriched in these two groups; MCS1 microbiota were characteristically dominated by Pseudomonadaceae which typically co-occurred with Sphingomonadaceae and Prevotellaceae. The second, larger group, exhibited a reciprocal gradient of Streptococcaceae or Prevotellaceae domination, which were designated MCS2A and MCS2B, respectively. Streptococcaceae-dominated MCS2A communities co-associated with Prevotellaceae and Veillonellaceae, and Prevotellaceae-dominated MCS2B assemblages with Veillonellaceae and Streptococcaceae. These distinct microbial states exhibited significant differences in diversity, with MCS1 exhibiting the lowest mean diversity compared to MCS2A or MCS2B communities (Faith's Phylogenetic diversity; one-way ANOVA, p<0.001, FIG. 2).

Using dominant family to classify samples, PCoA-ordination of weighted UniFrac distance matrices confirmed a strong and significant relationship between MCS class and bacterial beta-diversity (PERMANOVA, R²=0.670, p<0.001), corroborating the existence of compositionally distinct microbial states. Removal of the dominant family reads and re-application of DMM to the remaining data yielded the same two groups (n=46, n=136), indicating that dominant family is not the sole defining feature of these airway microbiota.

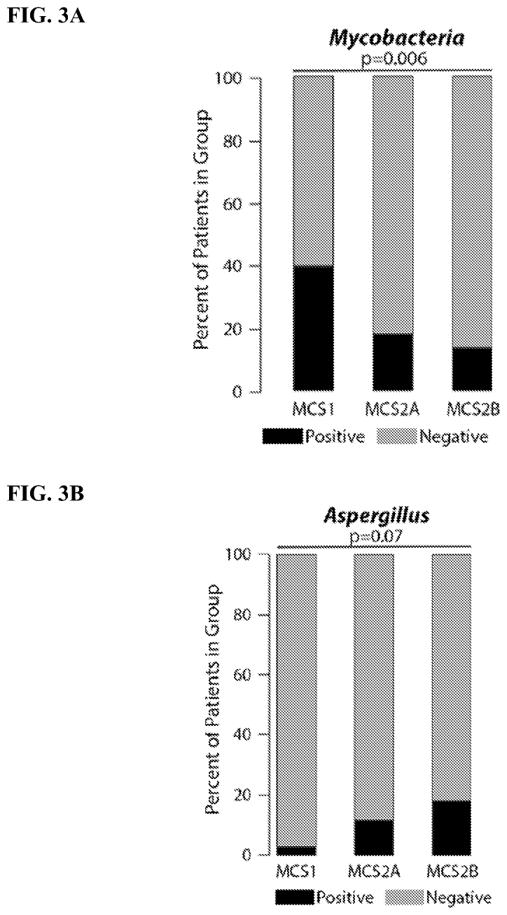

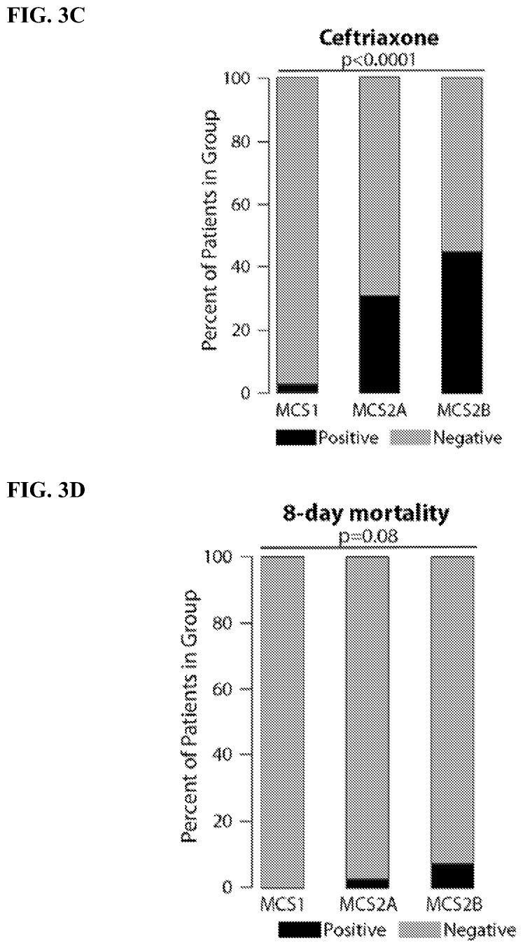

Lower airway state is related to demographic, clinical and microbiological factors. Next, it was asked whether the specific factors that explained the variation in bacterial beta-diversity were differentially associated with microbial state (Table 2). Neither gender nor alcohol consumption significantly differed across groups; however, MCS1 communities had significantly higher Mycobacterium detection (Chi-squared, p=0.006; FIG. 3A), while MCS2B communities exhibited increased culturable Aspergillus (Chi-squared, p=0.07; FIG. 3B). In parallel, the MCS2B group had significantly increased ceftriaxone administered (n=29/65), whereas MCS1 patients were almost never treated with ceftriaxone (n=1/36, Chi-squared, p<0.0001; FIG. 3C). Since ceftriaxone administration may be reflective of infection severity, variables associated with disease severity upon study enrollment were compared (e.g. fever, sputum production, chest pain, etc.) between ceftriaxone-treated and untreated patients; however, no statistically significant differences were observed.

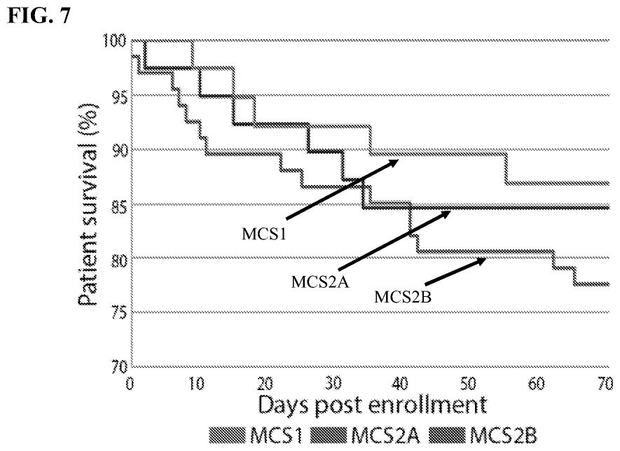

Mortality was tracked from enrollment through 70-days post-bronchoscopy. MCS2B patients exhibited the most deaths at 1-week post-enrollment (n=5/67) while MCS1 patients all survived (p=0.08; FIG. 3D). At 70-days, MCS2B patients still had the highest mortality (22%), followed by MCS2A (16%) and MCS1 patients (13%), though this trend did not reach significance (log-rank test, p>0.05, FIG. 7).

Airway microbial states are predicted to encode functionally distinct metagenome. The metagenomic content of each microbial state was predicted next using the Phylogenetic Investigation of Communities by Reconstruction of Unobserved States [PICRUSt (19)] package. Each microbial state was predicted to encode significantly distinct metagenomes and enriched for a characteristic set of gene pathways (PERMANOVA; R2=0.10, p<0.001). A total of 238 Kyoto Encyclopedia of Genes and Genomes [KEGG (27, 28)] pathways differed significantly between the three groups (Kruskal-Wallis, 329 pathways tested, q<0.05, Table 4). Despite decreased community diversity, MCS1 communities were predicted to encode a greater range of functional pathways compared to the other groups (Kruskal-Wallis, pairwise, p<0.001, q<0.05). This group was predicted to be significantly enriched for a broad range of pathways involved in 13-lactam, linoleic and arachidonic acid, and tryptophan metabolism; the majority of which (69%) were encoded by Pseudomonadaceae in these communities. MCS2A bacterial communities were enriched for pathways involved in biosynthesis of flavonols and ion channels, while MCS2B bacterial communities encoded glycan metabolism and glycosphingolipid biosynthesis pathways and lacked type II polyketide biosynthesis. Few pathways were predicted to be significantly enriched in MCS2B, indicating that associated increased mortality risk may be either driven by differential expression of pathways shared across compositionally distinct communities, or that non-bacterial species such as Aspergillus, detected with greater frequency in this microbial state, contribute substantially to their associated pathogenesis

TABLE 4
PICRUSt pathways predicted to be enriched in each microbial state.

| Enriched | | | | | Bonferroni |
|---|---|---|---|---|---|
| Group | Pathway | Sub Pathway | Super Pathway | p-value | p-value |
| MCS1 | Apoptosis | Cell Growth and Death | Cellular Processes | 9.30E−22 | 3.00E−19 |
| | Cell cycle - Caulobacter | Cell Growth and Death | Cellular Processes | 1.60E−15 | 5.30E−13 |
| | Meiosis - yeast | Cell Growth and Death | Cellular Processes | 2.80E−11 | 9.30E−09 |
| | p53 signaling pathway | Cell Growth and Death | Cellular Processes | 3.10E−18 | 1.00E−15 |
| | Bacterial chemotaxis | Cell Motility | Cellular Processes | 5.50E−19 | 1.80E−16 |
| | Bacterial motility proteins | Cell Motility | Cellular Processes | 9.90E−19 | 3.20E−16 |
| | Cytoskeleton proteins | Cell Motility | Cellular Processes | 3.30E−19 | 1.10E−16 |
| | Flagellar assembly | Cell Motility | Cellular Processes | 1.20E−18 | 3.80E−16 |
| | Endocytosis | Transport and Catabolism | Cellular Processes | 3.80E−09 | 1.30E−06 |
| | Peroxisome | Transport and Catabolism | Cellular Processes | 7.70E−18 | 2.50E−15 |
| | ABC transporters | Membrane Transport | Environmental Information | 4.30E−20 | 1.40E−17 |
| | | | Processing | | |
| | Bacterial secretion system | Membrane Transport | Environmental Information | 5.20E−19 | 1.70E−16 |
| | | | Processing | | |
| | Secretion system | Membrane Transport | Environmental Information | 7.70E−21 | 2.50E−18 |
| | | | Processing | | |
| | Transporters | Membrane Transport | Environmental Information | 1.10E−21 | 3.60E−19 |
| | | | Processing | | |
| | MAPK signaling pathway - yeast | Signal Transduction | Environmental Information | 6.30E−18 | 2.10E−15 |
| | | | Processing | | |
| | Phosphatidylinositol signaling | Signal Transduction | Environmental Information | 1.10E−21 | 3.50E−19 |
| | system | | Processing | | |
| | Two-component system | Signal Transduction | Environmental Information | 2.30E−20 | 7.40E−18 |
| | | | Processing | | |
| | Bacterial toxins | Signaling Molecules and | Environmental Information | 7.30E−12 | 2.40E−09 |
| | | Interaction | Processing | | |
| | Cellular antigens | Signaling Molecules and | Environmental Information | 3.10E−22 | 1.00E−19 |
| | | Interaction | Processing | | |
| | Chaperones and folding | Folding, Sorting and Degradation | Genetic Information Processing | 1.40E−19 | 4.70E−17 |
| | catalysts | | | | |
| | Proteasome | Folding, Sorting and Degradation | Genetic Information Processing | 1.60E−16 | 5.30E−14 |
| | Protein export | Folding, Sorting and Degradation | Genetic Information Processing | 4.00E−06 | 1.30E−03 |
| | Protein processing in | Folding, Sorting and Degradation | Genetic Information Processing | 1.50E−13 | 4.90E−11 |
| | endoplasmic reticulum | | | | |
| | RNA degradation | Folding, Sorting and Degradation | Genetic Information Processing | 9.90E−18 | 3.20E−15 |
| | Sulfur relay system | Folding, Sorting and Degradation | Genetic Information Processing | 4.20E−19 | 1.40E−16 |
| | Ubiquitin system | Folding, Sorting and Degradation | Genetic Information Processing | 7.80E−15 | 2.50E−12 |
| | Base excision repair | Replication and Repair | Genetic Information Processing | 1.40E−17 | 4.50E−15 |
| | Chromosome | Replication and Repair | Genetic Information Processing | 8.00E−18 | 2.60E−15 |
| | DNA repair and | Replication and Repair | Genetic Information Processing | 7.00E−15 | 2.30E−12 |
| | recombination proteins | | | | |
| | DNA replication | Replication and Repair | Genetic Information Processing | 1.80E−05 | 6.00E−03 |
| | DNA replication proteins | Replication and Repair | Genetic Information Processing | 1.50E−06 | 5.00E−04 |
| | Mismatch repair | Replication and Repair | Genetic Information Processing | 1.60E−05 | 5.40E−03 |
| | Non-homologous end-joining | Replication and Repair | Genetic Information Processing | 1.90E−18 | 6.20E−16 |
| | Nucleotide excision repair | Replication and Repair | Genetic Information Processing | 7.60E−09 | 2.50E−06 |
| | Transcription factors | Transcription | Genetic Information Processing | 4.20E−22 | 1.40E−19 |
| | Transcription machinery | Transcription | Genetic Information Processing | 8.90E−20 | 2.90E−17 |
| | Ribosome Biogenesis | Translation | Genetic Information Processing | 1.50E−18 | 4.80E−16 |
| | Ribosome biogenesis in | Translation | Genetic Information Processing | 1.40E−17 | 4.50E−15 |
| | eukaryotes | | | | |
| | Bladder cancer | Cancers | Human Diseases | 6.70E−13 | 2.20E−10 |
| | Colorectal cancer | Cancers | Human Diseases | 3.10E−18 | 1.00E−15 |
| | Pathways in cancer | Cancers | Human Diseases | 1.40E−19 | 4.50E−17 |
| | Prostate cancer | Cancers | Human Diseases | 2.50E−15 | 8.00E−13 |
| | Renal cell carcinoma | Cancers | Human Diseases | 1.10E−18 | 3.70E−16 |
| | Small cell lung cancer | Cancers | Human Diseases | 3.00E−18 | 9.80E−16 |
| | Hypertrophic cardiomyopathy | Cardiovascular Diseases | Human Diseases | 3.90E−16 | 1.30E−13 |
| | (HCM) | | | | |
| | Viral myocarditis | Cardiovascular Diseases | Human Diseases | 3.10E−18 | 1.00E−15 |
| | Primary immunodeficiency | Immune System Diseases | Human Diseases | 3.30E−13 | 1.10E−10 |
| | Systemic lupus erythematosus | Immune System Diseases | Human Diseases | 2.30E−17 | 7.40E−15 |
| | African trypanosomiasis | Infectious Diseases | Human Diseases | 2.50E−18 | 8.30E−16 |
| | Chagas disease (American | Infectious Diseases | Human Diseases | 6.10E−18 | 2.00E−15 |
| | trypanosomiasis) | | | | |
| | Epithelial cell signaling in | Infectious Diseases | Human Diseases | 1.10E−12 | 3.60E−10 |
| | Helicobacter pylori infection | | | | |
| | Influenza A | Infectious Diseases | Human Diseases | 3.10E−18 | 1.00E−15 |
| | Pertussis | Infectious Diseases | Human Diseases | 2.80E−18 | 9.20E−16 |
| | Toxoplasmosis | Infectious Diseases | Human Diseases | 3.10E−18 | 1.00E−15 |
| | Tuberculosis | Infectious Diseases | Human Diseases | 4.20E−15 | 1.40E−12 |
| | Vibrio cholerae infection | Infectious Diseases | Human Diseases | 1.50E−13 | 4.90E−11 |
| | Vibrio cholerae pathogenic | Infectious Diseases | Human Diseases | 4.00E−19 | 1.30E−16 |
| | cycle | | | | |
| | Type I diabetes mellitus | Metabolic Diseases | Human Diseases | 7.50E−05 | 2.50E−02 |
| | Type II diabetes mellitus | Metabolic Diseases | Human Diseases | 5.70E−18 | 1.90E−15 |
| | Alzheimer's disease | Neurodegenerative Diseases | Human Diseases | 1.10E−18 | 3.60E−16 |
| | Amyotrophic lateral sclerosis | Neurodegenerative Diseases | Human Diseases | 2.00E−18 | 6.50E−16 |
| | (ALS) | | | | |
| | Huntington's disease | Neurodegenerative Diseases | Human Diseases | 6.50E−21 | 2.10E−18 |
| | Parkinson's disease | Neurodegenerative Diseases | Human Diseases | 3.80E−19 | 1.30E−16 |
| | Prion diseases | Neurodegenerative Diseases | Human Diseases | 4.20E−16 | 1.40E−13 |
| | Alanine, aspartate and | Amino Acid Metabolism | Metabolism | 1.30E−18 | 4.30E−16 |
| | glutamate metabolism | | | | |
| | Amino acid related enzymes | Amino Acid Metabolism | Metabolism | 1.10E−16 | 3.80E−14 |
| | Arginine and proline | Amino Acid Metabolism | Metabolism | 7.50E−19 | 2.50E−16 |
| | metabolism | | | | |
| | Cysteine and methionine | Amino Acid Metabolism | Metabolism | 2.10E−17 | 6.80E−15 |
| | metabolism | | | | |
| | Glycine, serine and threonine | Amino Acid Metabolism | Metabolism | 1.50E−18 | 5.00E−16 |
| | metabolism | | | | |
| | Histidine metabolism | Amino Acid Metabolism | Metabolism | 9.00E−18 | 2.90E−15 |
| | Lysine biosynthesis | Amino Acid Metabolism | Metabolism | 1.70E−17 | 5.60E−15 |
| | Lysine degradation | Amino Acid Metabolism | Metabolism | 2.00E−18 | 6.60E−16 |
| | Phenylalanine metabolism | Amino Acid Metabolism | Metabolism | 1.30E−18 | 4.30E−16 |
| | Phenylalanine, tyrosine and | Amino Acid Metabolism | Metabolism | 2.70E−17 | 8.90E−15 |
| | tryptophan biosynthesis | | | | |
| | Tryptophan metabolism | Amino Acid Metabolism | Metabolism | 1.30E−18 | 4.40E−16 |
| | Tyrosine metabolism | Amino Acid Metabolism | Metabolism | 2.80E−19 | 9.10E−17 |
| | Valine, leucine and isoleucine | Amino Acid Metabolism | Metabolism | 6.00E−21 | 2.00E−18 |
| | biosynthesis | | | | |
| | Valine, leucine and isoleucine | Amino Acid Metabolism | Metabolism | 4.20E−18 | 1.40E−15 |
| | degradation | | | | |
| | beta-Lactam resistance | Biosynthesis of Other Secondary | Metabolism | 5.60E−15 | 1.80E−12 |
| | | Metabolites | | | |
| | Betalain biosynthesis | Biosynthesis of Other Secondary | Metabolism | 1.10E−07 | 3.50E−05 |
| | | Metabolites | | | |
| | Caffeine metabolism | Biosynthesis of Other Secondary | Metabolism | 1.10E−09 | 3.70E−07 |
| | | Metabolites | | | |
| | Flavonoid biosynthesis | Biosynthesis of Other Secondary | Metabolism | 1.20E−13 | 3.90E−11 |
| | | Metabolites | | | |
| | Isoflavonoid biosynthesis | Biosynthesis of Other Secondary | Metabolism | 5.30E−05 | 1.80E−02 |
| | | Metabolites | | | |
| | Isoquinoline alkaloid | Biosynthesis of Other Secondary | Metabolism | 6.40E−21 | 2.10E−18 |
| | biosynthesis | Metabolites | | | |
| | Novobiocin biosynthesis | Biosynthesis of Other Secondary | Metabolism | 5.60E−20 | 1.80E−17 |
| | | Metabolites | | | |
| | Penicillin and cephalosporin | Biosynthesis of Other Secondary | Metabolism | 1.10E−17 | 3.70E−15 |
| | biosynthesis | Metabolites | | | |
| | Phenylpropanoid biosynthesis | Biosynthesis of Other Secondary | Metabolism | 7.70E−17 | 2.50E−14 |
| | | Metabolites | | | |
| | Stilbenoid, diarylheptanoid | Biosynthesis of Other Secondary | Metabolism | 9.30E−15 | 3.10E−12 |
| | and gingerol biosynthesis | Metabolites | | | |
| | Streptomycin biosynthesis | Biosynthesis of Other Secondary | Metabolism | 4.20E−15 | 1.40E−12 |
| | | Metabolites | | | |
| | Tropane, piperidine and | Biosynthesis of Other Secondary | Metabolism | 1.30E−20 | 4.20E−18 |
| | pyridine alkaloid biosynthesis | Metabolites | | | |
| | Amino sugar and nucleotide | Carbohydrate Metabolism | Metabolism | 8.10E−12 | 2.70E−09 |
| | sugar metabolism | | | | |
| | Ascorbate and aldarate | Carbohydrate Metabolism | Metabolism | 2.90E−17 | 9.60E−15 |
| | metabolism | | | | |
| | Butanoate metabolism | Carbohydrate Metabolism | Metabolism | 2.70E−19 | 8.80E−17 |
| | C5-Branched dibasic acid | Carbohydrate Metabolism | Metabolism | 3.60E−19 | 1.20E−16 |
| | metabolism | | | | |
| | Citrate cycle | Carbohydrate Metabolism | Metabolism | 9.00E−19 | 2.90E−16 |
| | (TCA cycle) | | | | |
| | Fructose and mannose | Carbohydrate Metabolism | Metabolism | 9.20E−14 | 3.00E−11 |
| | metabolism | | | | |
| | Glycolysis/Gluconeogenesis | Carbohydrate Metabolism | Metabolism | 4.90E−19 | 1.60E−16 |
| | Glyoxylate and dicarboxylate | Carbohydrate Metabolism | Metabolism | 2.50E−18 | 8.20E−16 |
| | metabolism | | | | |
| | Inositol phosphate metabolism | Carbohydrate Metabolism | Metabolism | 5.30E−20 | 1.70E−17 |
| | Pentose and glucuronate | Carbohydrate Metabolism | Metabolism | 5.20E−18 | 1.70E−15 |
| | interconversions | | | | |
| | Pentose phosphate pathway | Carbohydrate Metabolism | Metabolism | 3.30E−18 | 1.10E−15 |
| | Propanoate metabolism | Carbohydrate Metabolism | Metabolism | 1.80E−19 | 6.00E−17 |
| | Pyruvate metabolism | Carbohydrate Metabolism | Metabolism | 2.50E−21 | 8.40E−19 |
| | Starch and sucrose | Carbohydrate Metabolism | Metabolism | 1.10E−09 | 3.50E−07 |
| | metabolism | | | | |
| | Carbon fixation in | Energy Metabolism | Metabolism | 5.60E−12 | 1.80E−09 |
| | photosynthetic organisms | | | | |
| | Carbon fixation pathways in | Energy Metabolism | Metabolism | 1.00E−18 | 3.30E−16 |
| | prokaryotes | | | | |
| | Methane metabolism | Energy Metabolism | Metabolism | 2.50E−17 | 8.20E−15 |
| | Nitrogen metabolism | Energy Metabolism | Metabolism | 3.70E−19 | 1.20E−16 |
| | Oxidative phosphorylation | Energy Metabolism | Metabolism | 4.80E−19 | 1.60E−16 |
| | Sulfur metabolism | Energy Metabolism | Metabolism | 1.20E−18 | 4.00E−16 |
| | Cytochrome P450 | Enzyme Families | Metabolism | 2.20E−10 | 7.10E−08 |
| | Peptidases | Enzyme Families | Metabolism | 3.30E−18 | 1.10E−15 |
| | Protein kinases | Enzyme Families | Metabolism | 2.40E−21 | 7.80E−19 |
| | Glycosyltransferases | Glycan Biosynthesis and | Metabolism | 4.90E−18 | 1.60E−15 |
| | | Metabolism | | | |
| | Lipopolysaccharide | Glycan Biosynthesis and | Metabolism | 2.20E−19 | 7.10E−17 |
| | biosynthesis | Metabolism | | | |
| | Lipopolysaccharide | Glycan Biosynthesis and | Metabolism | 8.40E−22 | 2.70E−19 |
| | biosynthesis proteins | Metabolism | | | |
| | Peptidoglycan biosynthesis | Glycan Biosynthesis and | Metabolism | 5.80E−06 | 1.90E−03 |
| | | Metabolism | | | |
| | alpha-Linolenic acid | Lipid Metabolism | Metabolism | 1.20E−17 | 4.10E−15 |
| | metabolism | | | | |
| | Arachidonic acid metabolism | Lipid Metabolism | Metabolism | 1.10E−20 | 3.70E−18 |
| | Biosynthesis of unsaturated | Lipid Metabolism | Metabolism | 1.10E−18 | 3.70E−16 |
| | fatty acids | | | | |
| | Ether lipid metabolism | Lipid Metabolism | Metabolism | 1.50E−14 | 5.00E−12 |
| | Fatty acid biosynthesis | Lipid Metabolism | Metabolism | 4.00E−21 | 1.30E−18 |
| | Fatty acid metabolism | Lipid Metabolism | Metabolism | 2.30E−18 | 7.40E−16 |
| | Glycerolipid metabolism | Lipid Metabolism | Metabolism | 5.10E−22 | 1.70E−19 |
| | Glycerophospholipid | Lipid Metabolism | Metabolism | 2.10E−18 | 7.00E−16 |
| | metabolism | | | | |
| | Linoleic acid metabolism | Lipid Metabolism | Metabolism | 1.30E−18 | 4.30E−16 |
| | Lipid biosynthesis proteins | Lipid Metabolism | Metabolism | 5.90E−18 | 1.90E−15 |
| | Primary bile acid biosynthesis | Lipid Metabolism | Metabolism | 4.80E−17 | 1.60E−14 |
| | Steroid biosynthesis | Lipid Metabolism | Metabolism | 3.20E−15 | 1.10E−12 |
| | Steroid hormone biosynthesis | Lipid Metabolism | Metabolism | 3.80E−18 | 1.20E−15 |
| | Synthesis and degradation of | Lipid Metabolism | Metabolism | 6.60E−20 | 2.20E−17 |
| | ketone bodies | | | | |
| | Biotin metabolism | Metabolism of Cofactors and | Metabolism | 3.70E−18 | 1.20E−15 |
| | | Vitamins | | | |
| | Folate biosynthesis | Metabolism of Cofactors and | Metabolism | 8.50E−18 | 2.80E−15 |
| | | Vitamins | | | |
| | Lipoic acid metabolism | Metabolism of Cofactors and | Metabolism | 2.60E−19 | 8.50E−17 |
| | | Vitamins | | | |
| | Nicotinate and nicotinamide | Metabolism of Cofactors and | Metabolism | 1.30E−19 | 4.40E−17 |
| | metabolism | Vitamins | | | |
| | One carbon pool by folate | Metabolism of Cofactors and | Metabolism | 8.80E−16 | 2.90E−13 |
| | | Vitamins | | | |
| | Pantothenate and CoA | Metabolism of Cofactors and | Metabolism | 1.00E−15 | 3.30E−13 |
| | biosynthesis | Vitamins | | | |
| | Porphyrin and chlorophyll | Metabolism of Cofactors and | Metabolism | 4.10E−19 | 1.30E−16 |
| | metabolism | Vitamins | | | |
| | Retinol metabolism | Metabolism of Cofactors and | Metabolism | 8.80E−18 | 2.90E−15 |
| | | Vitamins | | | |
| | Riboflavin metabolism | Metabolism of Cofactors and | Metabolism | 2.30E−21 | 7.60E−19 |
| | | Vitamins | | | |
| | Thiamine metabolism | Metabolism of Cofactors and | Metabolism | 1.40E−13 | 4.60E−11 |
| | | Vitamins | | | |
| | Ubiquinone and other | Metabolism of Cofactors and | Metabolism | 3.10E−22 | 1.00E−19 |
| | terpenoid-quinone | Vitamins | | | |
| | biosynthesis | | | | |
| | Vitamin B6 metabolism | Metabolism of Cofactors and | Metabolism | 1.30E−17 | 4.40E−15 |
| | | Vitamins | | | |
| | beta-Alanine metabolism | Metabolism of Other Amino Acids | Metabolism | 1.70E−18 | 5.70E−16 |
| | Cyanoamino acid metabolism | Metabolism of Other Amino Acids | Metabolism | 1.50E−18 | 4.80E−16 |
| | D-Arginine and D-ornithine | Metabolism of Other Amino Acids | Metabolism | 9.10E−07 | 3.00E−04 |
| | metabolism | | | | |
| | D-Glutamine and D-glutamate | Metabolism of Other Amino Acids | Metabolism | 5.40E−16 | 1.80E−13 |
| | metabolism | | | | |
| | Glutathione metabolism | Metabolism of Other Amino Acids | Metabolism | 3.00E−19 | 9.90E−17 |
| | Phosphonate and phosphinate | Metabolism of Other Amino Acids | Metabolism | 1.50E−22 | 4.90E−20 |
| | metabolism | | | | |
| | Selenocompound metabolism | Metabolism of Other Amino Acids | Metabolism | 2.00E−15 | 6.60E−13 |
| | Taurine and hypotaurine | Metabolism of Other Amino Acids | Metabolism | 5.70E−18 | 1.90E−15 |
| | metabolism | | | | |
| | Biosynthesis of siderophore | Metabolism of Terpenoids and | Metabolism | 3.00E−19 | 9.80E−17 |
| | group nonribosomal peptides | Polyketides | | | |
| | Biosynthesis of type II | Metabolism of Terpenoids and | Metabolism | 1.10E−16 | 3.50E−14 |
| | polyketide products | Polyketides | | | |
| | Carotenoid biosynthesis | Metabolism of Terpenoids and | Metabolism | 1.40E−15 | 4.70E−13 |
| | | Polyketides | | | |
| | Geraniol degradation | Metabolism of Terpenoids and | Metabolism | 7.10E−18 | 2.30E−15 |
| | | Polyketides | | | |
| | Limonene and pinene | Metabolism of Terpenoids and | Metabolism | 9.10E−19 | 3.00E−16 |
| | degradation | Polyketides | | | |
| | Polyketide sugar unit | Metabolism of Terpenoids and | Metabolism | 6.20E−11 | 2.00E−08 |
| | biosynthesis | Polyketides | | | |
| | Prenyltransferases | Metabolism of Terpenoids and | Metabolism | 4.90E−10 | 1.60E−07 |
| | | Polyketides | | | |
| | Terpenoid backbone | Metabolism of Terpenoids and | Metabolism | 8.10E−14 | 2.60E−11 |
| | biosynthesis | Polyketides | | | |
| | Tetracycline biosynthesis | Metabolism of Terpenoids and | Metabolism | 2.80E−24 | 9.10E−22 |
| | | Polyketides | | | |
| | Purine metabolism | Nucleotide Metabolism | Metabolism | 9.50E−16 | 3.10E−13 |
| | Aminobenzoate degradation | Xenobiotics Biodegradation and | Metabolism | 6.10E−19 | 2.00E−16 |
| | | Metabolism | | | |
| | Atrazine degradation | Xenobiotics Biodegradation and | Metabolism | 9.20E−19 | 3.00E−16 |
| | | Metabolism | | | |
| | Benzoate degradation | Xenobiotics Biodegradation and | Metabolism | 3.00E−19 | 9.70E−17 |
| | | Metabolism | | | |
| | Bisphenol degradation | Xenobiotics Biodegradation and | Metabolism | 4.30E−18 | 1.40E−15 |
| | | Metabolism | | | |
| | Caprolactam degradation | Xenobiotics Biodegradation and | Metabolism | 5.80E−19 | 1.90E−16 |
| | | Metabolism | | | |
| | Chloroalkane and | Xenobiotics Biodegradation and | Metabolism | 7.10E−20 | 2.30E−17 |
| | chloroalkene degradation | Metabolism | | | |
| | Chlorocyclohexane and | Xenobiotics Biodegradation and | Metabolism | 7.50E−18 | 2.50E−15 |
| | chlorobenzene degradation | Metabolism | | | |
| | Dioxin degradation | Xenobiotics Biodegradation and | Metabolism | 7.30E−18 | 2.40E−15 |
| | | Metabolism | | | |
| | Drug metabolism - cytochrome P450 | Xenobiotics Biodegradation and | Metabolism | 1.10E−18 | 3.70E−16 |
| | | Metabolism | | | |
| | Fluorobenzoate degradation | Xenobiotics Biodegradation and | Metabolism | 2.10E−17 | 6.90E−15 |
| | | Metabolism | | | |
| | Metabolism of xenobiotics by | Xenobiotics Biodegradation and | Metabolism | 7.80E−19 | 2.60E−16 |
| | cytochrome P450 | Metabolism | | | |
| | Naphthalene degradation | Xenobiotics Biodegradation and | Metabolism | 1.40E−19 | 4.50E−17 |
| | | Metabolism | | | |
| | Nitrotoluene degradation | Xenobiotics Biodegradation and | Metabolism | 6.30E−11 | 2.10E−08 |
| | | Metabolism | Metabolism | 1.10E−16 | 3.50E−14 |
| | Polycyclic aromatic | Xenobiotics Biodegradation and | | | |
| | hydrocarbon degradation | Metabolism | Metabolism | 6.70E−19 | 2.20E−16 |
| | Styrene degradation | Xenobiotics Biodegradation and | | | |
| | Toluene degradation | Metabolism | Metabolism | 1.50E−17 | 4.90E−15 |
| | | Xenobiotics Biodegradation and | | | |
| | | Metabolism | Metabolism | 2.00E−16 | 6.50E−14 |
| | Xylene degradation | Xenobiotics Biodegradation and | | | |
| | | Metabolism | Organismal Systems | 3.90E−19 | 1.30E−16 |
| | Cardiac muscle contraction | Circulatory System | | | |
| | Mineral absorption | Digestive System | Organismal Systems | 1.50E−12 | 4.90E−10 |
| | Adipocytokine signaling | Endocrine System | Organismal Systems | 2.30E−16 | 7.40E−14 |
| | pathway | | | | |
| | GnRH signaling pathway | Endocrine System | Organismal Systems | 3.80E−09 | 1.30E−06 |
| | Insulin signaling pathway | Endocrine System | Organismal Systems | 2.00E−14 | 6.50E−12 |
| | Melanogenesis | Endocrine System | Organismal Systems | 4.20E−08 | 1.40E−05 |
| | PPAR signaling pathway | Endocrine System | Organismal Systems | 1.10E−18 | 3.50E−16 |
| | Progesterone-mediated oocyte | Endocrine System | Organismal Systems | 2.30E−15 | 7.70E−13 |
| | maturation | | | | |
| | Renin-angiotensin system | Endocrine System | Organismal Systems | 1.60E−16 | 5.40E−14 |
| | Circadian rhythm - plant | Environmental Adaptation | Organismal Systems | 3.20E−15 | 1.10E−12 |
| | Plant-pathogen interaction | Environmental Adaptation | Organismal Systems | 1.30E−18 | 4.30E−16 |
| | Proximal tubule bicarbonate | Excretory System | Organismal Systems | 2.20E−17 | 7.30E−15 |
| | reclamation | | | | |
| | Antigen processing and | Immune System | Organismal Systems | 2.30E−15 | 7.70E−13 |
| | presentation | | | | |
| | Fc gamma R-mediated | Immune System | Organismal Systems | 3.80E−09 | 1.30E−06 |
| | phagocytosis | | | | |
| | Glutamatergic synapse | Nervous System | Organismal Systems | 2.30E−20 | 7.60E−18 |
| | Cell division | Cellular Processes and | Unclassified | 7.20E−20 | 2.40E−17 |
| | | Signaling | | | |
| | Cell motility and secretion | Cellular Processes and | Unclassified | 6.00E−19 | 2.00E−16 |
| | | Signaling | | | |
| | Inorganic ion transport and | Cellular Processes and | Unclassified | 1.00E−18 | 3.30E−16 |
| | metabolism | Signaling | | | |
| | Membrane and intracellular | Cellular Processes and | Unclassified | 4.40E−20 | 1.40E−17 |
| | structural molecules | Signaling | | | |
| | Other ion-coupled transporters | Cellular Processes and | Unclassified | 5.10E−19 | 1.70E−16 |
| | | Signaling | | | |
| | Other transporters | Cellular Processes and | Unclassified | 1.20E−18 | 4.00E−16 |
| | | Signaling | | | |
| | Pores ion channels | Cellular Processes and | Unclassified | 2.80E−19 | 9.20E−17 |
| | | Signaling | | | |
| | Signal transduction | Cellular Processes and | Unclassified | 9.20E−22 | 3.00E−19 |
| | mechanisms | Signaling | | | |
| | Protein folding and associated | Genetic Information | Unclassified | 1.70E−18 | 5.50E−16 |
| | processing | Processing | | | |
| | Replication, recombination | Genetic Information | Unclassified | 3.20E−17 | 1.00E−14 |
| | and repair proteins | Processing | | | |
| | Transcription related proteins | Genetic Information | Unclassified | 1.90E−21 | 6.10E−19 |
| | | Processing | | | |
| | Translation proteins | Genetic Information | Unclassified | 5.40E−17 | 1.80E−14 |
| | | Processing | | | |
| | Amino acid metabolism | Metabolism | Unclassified | 2.00E−17 | 6.40E−15 |
| | Biosynthesis and | Metabolism | Unclassified | 5.70E−19 | 1.90E−16 |
| | biodegradation of secondary | | | | |
| | metabolites | | | | |
| | Carbohydrate metabolism | Metabolism | Unclassified | 5.10E−20 | 1.70E−17 |
| | Energy metabolism | Metabolism | Unclassified | 5.00E−19 | 1.70E−16 |
| | Glycan biosynthesis and | Metabolism | Unclassified | 2.70E−18 | 8.80E−16 |
| | metabolism | | | | |
| | Lipid metabolism | Metabolism | Unclassified | 9.70E−20 | 3.20E−17 |
| | Metabolism of cofactors and | Metabolism | Unclassified | 1.10E−18 | 3.60E−16 |
| | vitamins | | | | |
| | Others | Metabolism | Unclassified | 2.30E−20 | 7.50E−18 |
| | Function unknown | Poorly Characterized | Unclassified | 4.10E−19 | 1.30E−16 |
| | General function prediction | Poorly Characterized | Unclassified | 2.30E−18 | 7.70E−16 |
| | only | | | | |
| MCS2A | Phosphotransferase system | Membrane Transport | Environmental Information | 4.40E−11 | 1.40E−08 |
| | | | Processing | | |
| | (PTS) | | | | |
| | Ion channels | Signaling Molecules and | Environmental Information | 3.10E−14 | 1.00E−11 |
| | | | Processing | | |
| | | Interaction | | | |
| | RNA polymerase | Transcription | Genetic Information Processing | 3.20E−08 | 1.00E−05 |
| | RNA transport | Translation | Genetic Information Processing | 1.50E−19 | 5.00E−17 |
| | Bacterial invasion of | Infectious Diseases | Human Diseases | 1.40E−05 | 4.40E−03 |
| | epithelial cells | Infectious Diseases | Human Diseases | 4.80E−18 | 1.60E−15 |
| | Staphylococcus aureus | | | | |
| | infection | | | | |
| | Flavone and flavonol | Biosynthesis of Other | Metabolism | 4.10E−13 | 1.30E−10 |
| | biosynthesis | Secondary | | | |
| | | Metabolites | | | |
| | Galactose metabolism | Carbohydrate Metabolism | Metabolism | 9.80E−10 | 3.20E−07 |
| | D-Alanine metabolism | Metabolism of Other Amino Acids | Metabolism | 5.60E−08 | 1.90E−05 |
| | Ethylbenzene degradation | Xenobiotics Biodegradation and | Metabolism | 4.10E−17 | 1.30E−14 |
| | Carbohydrate digestion and | Metabolism | Organismal Systems | 1.00E−11 | 3.40E−09 |
| | absorption | Digestive System | | | |
| | RIG-I-like receptor signaling | Immune System | Organismal Systems | 1.10E−04 | 3.60E−02 |
| | pathway | | | | |
| | Sporulation | Cellular Processes and | Unclassified | 2.80E−11 | 9.10E−09 |
| | | Signaling | | | |
| | Nucleotide metabolism | Metabolism | Unclassified | 1.50E−05 | 4.90E−03 |
| MCS2B | Lysosome | Transport and Catabolism | Cellular Processes | 1.00E−18 | 3.40E−16 |
| | Amoebiasis | Infectious Diseases | Human Diseases | 3.30E−11 | 1.10E−08 |
| | Glycosaminoglycan | Glycan Biosynthesis and | Metabolism | 6.20E−20 | 2.00E−17 |
| | degradation | Metabolism | | | |
| | Glycosphingolipid | Glycan Biosynthesis and | Metabolism | 1.90E−20 | 6.30E−18 |
| | biosynthesis - ganglio series | Metabolism | | | |
| | Glycosphingolipid | Glycan Biosynthesis and | Metabolism | 2.10E−20 | 6.80E−18 |
| | biosynthesis - globo series | Metabolism | | | |
| | N-Glycan biosynthesis | Glycan Biosynthesis and | Metabolism | 3.60E−18 | 1.20E−15 |
| | | Metabolism | | | |
| | Other glycan degradation | Glycan Biosynthesis and | Metabolism | 1.00E−16 | 3.40E−14 |
| | | Metabolism | | | |
| | Various types of N-glycan | Glycan Biosynthesis and | Metabolism | 2.50E−05 | 8.10E−03 |
| | biosynthesis | Metabolism | | | |
| | Sphingolipid metabolism | Lipid Metabolism | Metabolism | 3.70E−17 | 1.20E−14 |
| | Zeatin biosynthesis | Metabolism of Terpenoids and | Metabolism | 1.40E−18 | 4.50E−16 |
| | | Polyketides | | | |
| | Protein digestion and | Digestive System | Organismal Systems | 1.30E−20 | 4.40E−18 |
| | absorption | | | | |
| | NOD-like receptor signaling | Immune System | Organismal Systems | 2.20E−15 | 7.10E−13 |
| | pathway | | | | |
| | Restriction enzyme | Genetic Information Processing | Unclassified | 4.80E−18 | 1.60E−15 |

Microbial Community Types Induce Distinct and Characteristic Lower Airway Immune Responses.

RNA extracted from a subset of compositionally representative BAL samples (n=10/MCS) was used to analyze expression of a diverse panel of immune markers, chosen for their known associations with HIV, chronic bacterial infections, or airway inflammatory responses. mRNA expression levels (GAPDH-normalized) were used to generate a multivariate profile of host immune response. PERMANOVA analysis indicated that airway immune response was significantly related to the MCS present (PERMANOVA; R²=0.168, p<0.005). Specific immune responses were significantly enriched in particular MCS (one-way ANOVA, p<0.05). For example, MCS1 patients exhibited significantly higher expression of T-cell immunoglobulin and mucin domain 3 (TIM-3), a glycoprotein expressed by T and innate cells, that down-regulates T-helper 1 activity and pro-inflammatory responses (29), and plays a key role in T-cell dysfunction that occurs during chronic viral infection (30, 31).

By contrast, MCS2A demonstrated the lowest TIM-3 expression and significantly increased expression of protein-arginine deiminase type-4 (PADI4), which converts arginine to citrulline, an a-amino acid post-translationally incorporated into histones, filaggrin and proteins involved in myelination (32). Additionally, this MCS trended toward significantly higher levels of interleukin-10 (IL-10; anti-inflammatory cytokine) and programmed cell death protein 1 (PD-1; T cell negative regulator and exhaustion marker), and lower levels of forkhead box P3 (FOXP3; master regulator of regulatory T cells) expression. MCS2B subjects displayed increased interferon-alpha (IFNα), which characteristically protects against infection in immunocompetent subjects, but is associated with rapid disease progression in HIV-infection (33), IL-13 (mediator of T-helper 2 cell function), occludin/ELL Domain-Containing Protein 1 (OCEL1; maintains and regulates tight junctions), and protein tyrosine phosphatase receptor type C (PTPRC; expressed on micro-vesicles produced by HIV-infected cells) were also significantly increased. These patients also trended towards increased expression of IL-5 (mediator of T-helper 2 cell function which stimulates B cell growth), MUC5AC (the primary airway mucin), PD-1, and IL-33 (pro-inflammatory cytokine which induces Th2 responses), indicating a significant T-helper 2 skew.

Airway Microbial States are Associated with Distinct Circulating Metabolites.

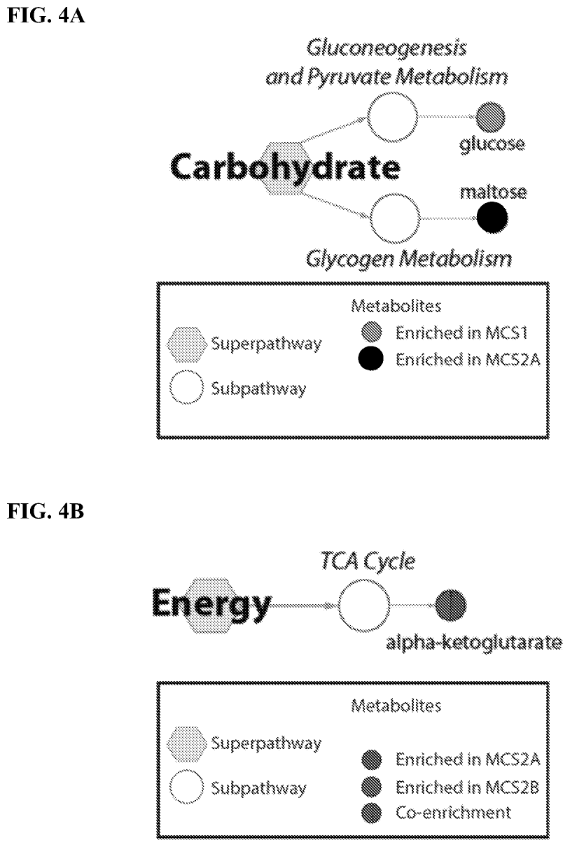

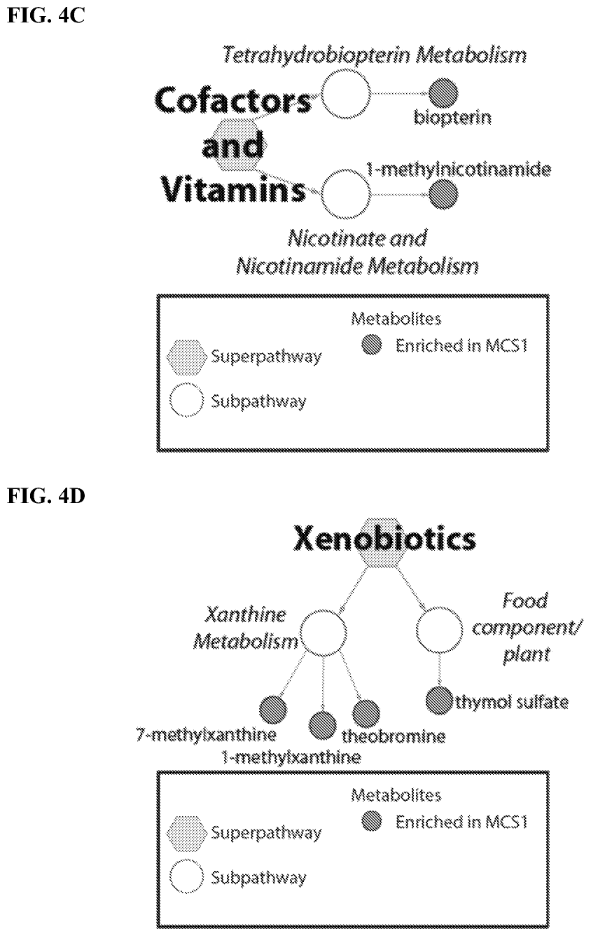

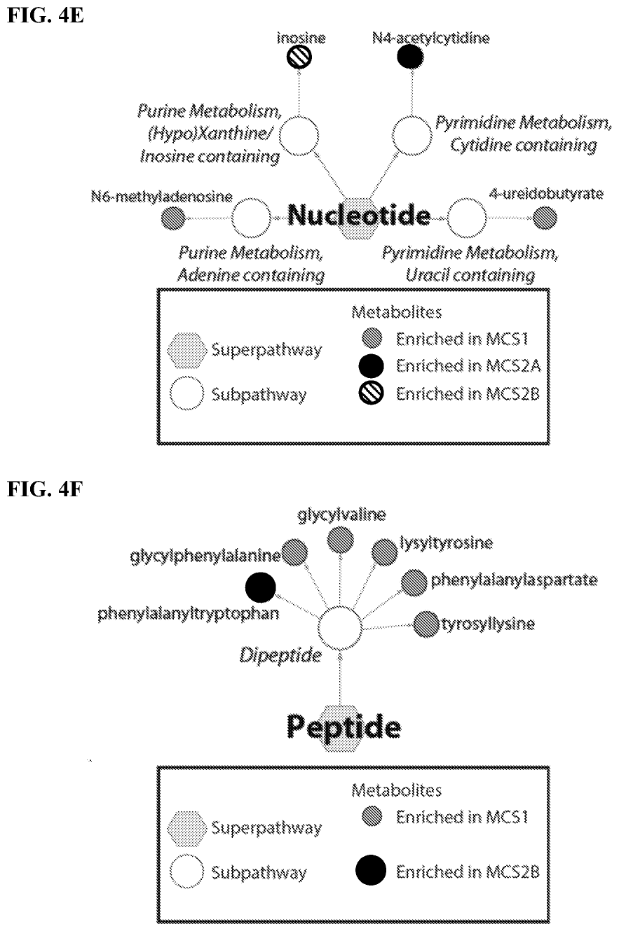

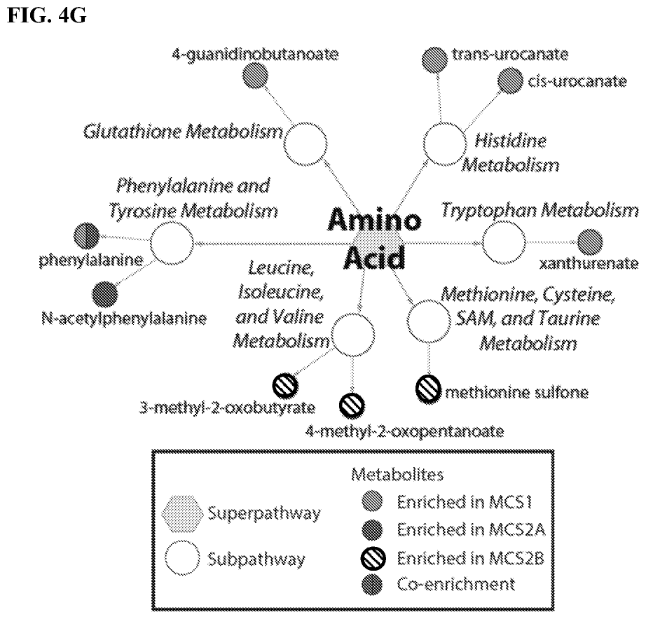

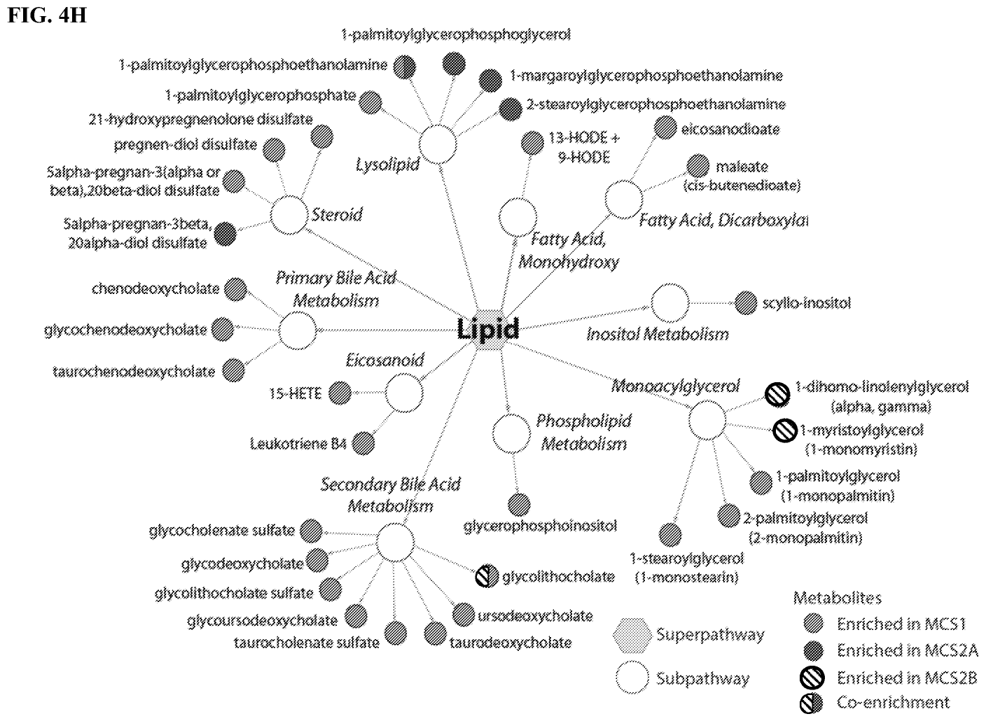

Paired serum from patients for whom BAL immune profiles were generated (n=30), were examined using Liquid and Gas Chromatography-Mass Spectrometry to determine whether distinct systemic metabolic profiles were associated with airway MCS. A total of 60 metabolites differed significantly between all three groups (Kruskal-Wallis, p<0.05; FIGS. 4A-4G and Table 5). As the in silico metagenomic analysis predicted, MCS1 patients were characterized by significant enrichment of xanthurenate (a tryptophan metabolite) and arachidonic acid metabolites, including the eicosanoids leukotriene B4, a pro-inflammatory lipid-mediator, and 15-HETE (15-hydroxyeicosatetraenoic acid), which induces pulmonary vasoconstriction and edema (34). In addition, MCS1 patients were significantly enriched for multiple products of primary and secondary bile metabolism (e.g. chenodeoxycholate, glycodeoxycholate, taurochenodeoxycholate, and ursodeoxycholate), several of which have been shown to upregulate inflammatory responses along with LPS (35), indicating that the activities of the gastrointestinal microbiome may also contribute to the tone of host inflammation in MCS1.

TABLE 5
Metabolites enriched in each microbial state.

| Enriched | | | | |
|---|---|---|---|---|
| group | Metabolite | Super pathway | Sub pathway | p-value |
| MCS1 | 4-guanidinobutanoate | Amino Acid | Guanidino and Acetamido Metabolism | 0.008 |
| | cis-urocanate | Amino Acid | Histidine Metabolism | 0.005 |
| | trans-urocanate | Amino Acid | Histidine Metabolism | 0.004 |
| | xanthurenate | Amino Acid | Tryptophan Metabolism | 0.048 |
| | glucose | Carbohydrate | Glycolysis, Gluconeogenesis, and | 0.002 |
| | | | Pyruvate Metabolism | |
| | 1-methylnicotinamide | Cofactors and Vitamins | Nicotinate and Nicotinamide Metabolism | 0.007 |
| | biopterin | Cofactors and Vitamins | Tetrahydrobiopterin Metabolism | 0.043 |
| | 15-HETE | Lipid | Eicosanoid | 0.016 |
| | leukotriene B4 | Lipid | Eicosanoid | 0.02 |
| | eicosanodioate | Lipid | Fatty Acid, Dicarboxylate | 0.048 |
| | maleate (cis-Butenedioate) | Lipid | Fatty Acid, Dicarboxylate | 0.044 |
| | 13-HODE + 9-HODE | Lipid | Fatty Acid, Monohydroxy | 0.017 |
| | scyllo-inositol | Lipid | Inositol Metabolism | 0.038 |
| | 1-palmitoylglycerophosphate | Lipid | Lysolipid | 0.043 |
| | 1-palmitoylglycerol (1-monopalmitin) | Lipid | Monoacylglycerol | 0.038 |
| | 1-stearoylglycerol (1-monostearin) | Lipid | Monoacylglycerol | 0.046 |
| | 2-palmitoylglycerol (2-monopalmitin) | Lipid | Monoacylglycerol | 0.02 |
| | glycerophosphoinositol | Lipid | Phospholipid Metabolism | 0.016 |
| | chenodeoxycholate | Lipid | Primary Bile Acid Metabolism | 0.028 |
| | glycochenodeoxycholate | Lipid | Primary Bile Acid Metabolism | 0.022 |
| | taurochenodeoxycholate | Lipid | Primary Bile Acid Metabolism | 0.039 |
| | glycocholenate sulfate | Lipid | Secondary Bile Acid Metabolism | 0.017 |
| | glycodeoxycholate | Lipid | Secondary Bile Acid Metabolism | 0.041 |
| | glycolithocholate sulfate | Lipid | Secondary Bile Acid Metabolism | 0.021 |
| | glycoursodeoxycholate | Lipid | Secondary Bile Acid Metabolism | 0.016 |
| | taurocholenate sulfate | Lipid | Secondary Bile Acid Metabolism | 0.05 |
| | taurodeoxycholate | Lipid | Secondary Bile Acid Metabolism | 0.013 |
| | ursodeoxycholate | Lipid | Secondary Bile Acid Metabolism | 0.03 |
| | 21-hydroxypregnenolone disulfate | Lipid | Steroid | 0.012 |
| | 5alpha-pregnan-3(alpha or beta), 20beta- | Lipid | Steroid | 0.038 |
| | diol disulfate | | | |
| | pregnen-diol disulfate | Lipid | Steroid | 0.04 |
| | N6-methyladenosine | Nucleotide | Purine Metabolism, Adenine containing | 0.031 |
| | 4-ureidobutyrate | Nucleotide | Pyrimidine Metabolism, Uracil | 0.044 |
| | | | containing | |
| | glycylphenylalanine | Peptide | Dipeptide | 0.03 |
| | glycylvaline | Peptide | Dipeptide | 0.039 |
| | lysyltyrosine | Peptide | Dipeptide | 0.006 |
| | phenylalanylaspartate | Peptide | Dipeptide | 0.005 |
| | tyrosyllysine | Peptide | Dipeptide | 0.002 |
| | thymol sulfate | Xenobiotics | Food Component/Plant | 0.017 |
| | 1 -methylxanthine | Xenobiotics | Xanthine Metabolism | 0.009 |
| | 7-methylxanthine | Xenobiotics | Xanthine Metabolism | 0.037 |
| | theobromine | Xenobiotics | Xanthine Metabolism | 0.046 |
| MCS2A | N-acetylphenylalanine | Amino Acid | Phenylalanine and Tyrosine Metabolism | 0.022 |
| | maltose | Carbohydrate | Glycogen Metabolism | 0.035 |
| | 1-margaroylglycerophosphoethanolamine | Lipid | Lysolipid | 0.008 |
| | 1-palmitoylglycerophosphoglycerol | Lipid | Lysolipid | 0.024 |
| | 2-stearoylglycerophosphoethanolamine | Lipid | Lysolipid | 0.015 |
| | 5alpha-pregnan-3beta, 20alpha-diol | Lipid | Steroid | 0.023 |
| | disulfate | | | |
| | N4-acetylcytidine | Nucleotide | Pyrimidine Metabolism, Cytidine | 0.028 |
| | | | containing | |
| MCS2B | 3-methyl-2-oxobutyrate | Amino Acid | Leucine, Isoleucine and Valine | 0.016 |
| | | | Metabolism | |
| | 4-methyl-2-oxopentanoate | Amino Acid | Leucine, Isoleucine and Valine | 0.044 |
| | | | Metabolism | |
| | methionine sulfone | Amino Acid | Methionine, Cysteine, SAM and Taurine | 0.043 |
| | | | Metabolism | |
| | 1-dihomo-linolenylglycerol (alpha, gamma) | Lipid | Monoacylglycerol | 0.028 |
| | 1-myristoylglycerol (1-monomyristin) | Lipid | Monoacylglycerol | 0.044 |
| | inosine | Nucleotide | Purine Metabolism, | 0.031 |
| | phenylalanyltryptophan | Peptide | (Hypo)Xanthine/Inosine containing | 0.047 |
| | | | Dipeptide | |
| MCS1 & | phenylalanine | Amino Acid | Phenylalanine and Tyrosine Metabolism | 0.024 |
| MCS2A | 1-palmitoylglycerophosphoethanolamine | Lipid | Lysolipid | 0.048 |
| MCS1 & | glycolithocholate | Lipid | Secondary Bile Acid Metabolism | 0.046 |
| MCS2B | | | | |
| MCS2A & | alpha-ketoglutarate | Energy | TCA Cycle | 0.036 |
| MCS2B | | | | |

As predicted, MCS2B patients were characterized by significantly reduced relative levels of circulating metabolites compared to MCS1 patients. However, significant increases in amino acid metabolites 3-methyl-2-oxobutyrate and 4-methyl-2-oxopentanoate (both involved in valine and leucine metabolism), monoacylglycerols associated with lipid metabolism (1-dihomo-linolenylglycerol and 1-myristoylglycerol), and inosine (purine metabolism) were significantly enriched. MCS2A patients exhibited a mixture of metabolites identified in the other MCS but at lower concentrations, with a unique increase in lysolipid and pyrimidine metabolism and a decrease in monoacylglycerols. Thus, products of several of the biosynthetic or metabolic pathways predicted to discriminate between patients with specific MCS were significantly and differentially enriched in their circulation.

To verify that specific MCS, their predicted metagenomes, local airway immune responses, and serum metabolites were inter-related, we applied Procrustes (13, 36) and Mantel (37) analyses. Both confirmed a strong and significant correlation between each of these data matrices [correlation between bacterial community composition and 1. PICRUSt metagenomic prediction (Procrustes: r²=0.513, p<0.001; Mantel: r²=0.674, p<0.001), or 2. Airway immune expression (Procrustes: r²=0.147, p<0.031; Mantel: r²=0.122, p=0.067), or 3. Serum metabolites (Procrustes: r²=0.414, p<0.001; Mantel r²=0.286, p<0.001)]. This result indicates that the HIV-infected pneumonia patients who possess distinct airway MCS exhibit corresponding features of immune dysfunction and a characteristic peripheral metabolome.

Immune Response and Mortality Risk Relate to Distinct Lung Microbiomes in HIV-Pneumonia Patients

Factors that influence pneumonia outcomes in HIV-infected patients are poorly defined—it was hypothesized that the airway microbiome may influence these outcomes. Three distinct microbial states were identified in this study; they exhibited significant differences in alpha diversity, culturable Aspergillus or Mycobacterium, ceftriaxone administration, immune responses, and metabolic signatures, and trended towards differences in mortality outcomes. Recent work by Cribbs and colleagues demonstrated HIV-infected patients are enriched for pneumonia-associated bacteria, including Streptococcus, even in the absence of airway infection, and exhibit a distinct metabolic microenvironment compared to healthy subjects (38). Together with this study, the data herein suggests that specific lower airway microbial states may lead to functionally relevant metabolic shifts that relate to distinct pathways of disease pathogenesis in HIV-infected individuals.

Segal and colleagues have shown that healthy individuals who have an enrichment of oral taxa, including Prevotella, within their lower airways, exhibit increased inflammatory cytokines and Th17 cells (39). This corroborates the findings herein that Prevotellaceae-dominated airway microbiota promote inflammation within the lower airways, including IL-17A expression.

Recent studies have demonstrated that composition of the airway microbiota influences susceptibility to Aspergillus infection (40), and that HIV-associated airway disease is related to fungal community alterations, including Aspergillus enrichment (41). The data herein supports these findings and suggests that Aspergillus prospers in a Prevotellaceae-dominated microbiota in the context of a Th2-skewed airway immune response. Several recent studies have confirmed the capacity of multiple Aspergillus species to induce Th2 responses (42), particularly in early-stage airway infection (43), suggesting that this species may not simply co-colonize MCS2B airways, but may actively define immunological responses characteristic of this patient sub-group. Patients with this airway microbiota were more likely to have been administered ceftriaxone and exhibited the highest mortality rates; one possible conclusion from these observations is that ceftriaxone administration selectively enriches for an MCS2B microbial community, and that their inter-kingdom microbial activities elicit a host immune response that increases mortality risk. However, the paucity of pre-antibiotic bronchoscopic samples, which are both ethically and logistically difficult to obtain, precludes definitive conclusions on whether ceftriaxone administration is responsible for the presence of this more severe MCS, or whether MCS2B assemblages pre-existed in these patients' airways prior to hospitalization. In embodiments, ceftriaxone administration predisposes a subject for a MCS2B microbial community. In embodiments, a subject who has not been administered ceftriaxone administration has MCS2B microbial community.

MC S1 patients, who exhibited the lowest levels of profiled immune activation markers, were predicted to be enriched for pathways involved in linoleic and arachidonic acid metabolism. Leukotriene B4, a product of arachidonic acid metabolism typically produced by leukocytes in response to inflammatory mediators, was detected in significantly increased concentrations in these patients' serum. While circulating leukotriene B4 in MCS1 patients is likely produced by leukocytes, the data herein suggests that microbial metabolism of arachidonic acid may contribute to their circulating leukotriene B4 and that microbial-derived lipid inflammation may underlie their immune dysfunction. Mycobacterium was more prevalent in MCS1 patients, who also exhibited a significant increase in TIM-3 expression. This is consistent with the findings of Behar and colleagues who demonstrated in vivo surface expression of Tim-3 on macrophages infected with M. tuberculosis (44).

MCS2A appears to represent an intermediate microbial state, between the MCS2B and MCS1 groups in terms of clinical associations, alpha-diversity, composition, metabolites, and immune expression. This raises the possibility that airway microbiota may be dynamic and transition through distinct microbiological states, particularly under antimicrobial selective pressure; however, large longitudinal studies are necessary to address this possibility. In embodiments, the airway microbiota is dynamic and transition through distinct microbiological states, particularly under antimicrobial selective pressure. Nonetheless, patients with MCS2A were uniquely characterized by increased lower airways PADI4 expression. Extracellular bronchial PADI4 has been shown to citrullinate the innate immune defensin human cathelicidin LL-37/human cationic antimicrobial protein-18, rendering it less efficient at neutralizing lipopolysaccharide. PADI4 is detected in the airways of patients with chronic obstructive pulmonary disease, who also exhibit impaired antibacterial response against Streptococcus (45), indicating that MCS2A patients, who exhibit expansion of Streptococcaceae and induction of PADI4, may also have diminished capacity to respond to the dominant bacterial family present in their airways.

Although lower airway colonization is considered uniformly detrimental to patients, the data herein show that specific, repeated airway microbiome states, discriminated upon the basis of microbial composition, function, host immune response, and clinical outcomes, exist in HIV-infected pneumonia patient subsets. Though the majority of patients fall into the three microbial states described, it is recognized that not all patients belong to these groupings, which is not surprising in light of recent work by Twigg and colleagues showing far greater variation across lower airway communities in advanced-HIV patients than healthy individuals (8). Larger cohorts of patients are necessary to sufficiently power studies examining other rarer microbial states and their immunological and clinical implications. This cohort provides insight into HIV-infected Ugandan pneumonia patients; however, these results may have limited applicability to patients in Western countries due to differences in patient demographics, laboratory testing and antibiotic availability, and high HIV-TB co-prevalence in Uganda. Furthermore, while BAL allows identification of general microbiota patterns within the lower airways, examining spatial-specific microbiota and their interactions with the host requires lung biopsies or brushings, which are beyond the scope of this study. While this study did not use paired oral-BAL samples to control for oral contamination, it was previously shown that oral and lower airway microbiota within HIV-infected pneumonia patients display niche specificity (7). The data herein trends towards a significant relationship between mortality and airway MCS. It is calculated that to achieve an eighty percent likelihood of detecting a significant difference in mortality (power=0.8), 100 patients per MCS would have to be studied, underscoring the need and utility of larger cohorts. Despite these limitations, this study identified several factors that shape microbial community composition in the lower airways of HIV-infected pneumonia patients. Moreover, it identifies distinct bacterial microbiota states that repeat over large numbers of patients and builds an argument that pneumonia patient heterogeneity, with respect to both immunological and clinical outcomes, may be related to compositional and functional differences in airway microbiomes.

### REFERENCES

### References for Example 1

1. The Gap Report: UNAIDS; 2014.
2. World Health Organization (WHO) UNAIDS Report on the Global AIDS Epidemic; 2013.
3. Badri M, Ehrlich R, Wood R, Pulerwitz T, Maartens G. Association between tuberculosis and HIV disease progression in a high tuberculosis prevalence area. Int J Tuberc Lung Dis 2001; 5: 225-232.
4. Chaisson R E, Martinson N A. Tuberculosis in Africa—combating an HIV-driven crisis. N Engl J Med 2008; 358: 1089-1092.
5. French N, Gordon S B, Mwalukomo T, White S A, Mwafulirwa G, Longwe H, Mwaiponya M, Zijlstra E E, Molyneux M E, Gilks C F. A trial of a 7-valent pneumococcal conjugate vaccine in HIV-infected adults. N Engl J Med 2010; 362: 812-822.
6. Marshall C S, Curtis A J, Spelman T, O'Brien D P, Greig J, Shanks L, du Cros P, Casas E C, da Fonseca M S, Athan E, Elliott J H. Impact of HIV-associated conditions on mortality in people commencing anti-retroviral therapy in resource limited settings. PLoS One 2013; 8: e68445.
7. Iwai S, Fei M, Huang D, Fong S, Subramanian A, Grieco K, Lynch S V, Huang L. Oral and airway microbiota in HIV-infected pneumonia patients. J Clin Microbiol 2012; 50: 29953002.
8. Twigg Iii H L, Knox K S, Zhou J, Crothers K A, Nelson D E, Toh E, Day R B, Lin H, Gao X, Dong Q, Mi D, Katz B P, Sodergren E, Weinstock G M. Effect of Advanced HIV Infection on the Respiratory Microbiome. American Journal of Respiratory and Critical Care Medicine 2016.
9. Flanagan J L, Brodie E L, Weng L, Lynch S V, Garcia O, Brown R, Hugenholtz P, DeSantis T Z, Andersen G L, Wiener-Kronish J P, Bristow J. Loss of bacterial diversity during antibiotic treatment of intubated patients colonized with Pseudomonas aeruginosa. J Clin Microbiol 2007; 45: 1954-1962.
10. Iwai S, Huang D, Fong S, Jarlsberg L G, Worodria W, Yoo S, Cattamanchi A, Davis J L, Kaswabuli S, Segal M, Huang L, Lynch S V. The lung microbiome of Ugandan HIV-infected pneumonia patients is compositionally and functionally distinct from that of San Franciscan patients. PLoS One 2014; 9: e95726.
11. Shenoy M, Iwai S, Lin D, Worodria W, Ayakaka I, Byanyima P, Kaswabuli S, Fong S, Stone S, Chang E, Davis J L, Faruqi A A, Segal M R, Huang L, Lynch S V. Title. American Thoracic Society International Conference (In press).
12. Maga T, Salzberg S L. FLASH: fast length adjustment of short reads to improve genome assemblies. Bioinformatics 2011; 27: 2957-2963.
13. Caporaso J G, Kuczynski J, Stombaugh J, Bittinger K, Bushman F D, Costello E K, Fierer N, Peria A G, Goodrich J K, Gordon J I, Huttley G A, Kelley S T, Knights D, Koenig J E, Ley R E, Lozupone C A, McDonald D, Muegge B D, Pirrung M, Reeder J, Sevinsky J R, Tumbaugh P J, Walters W A, Widmann J, Yatsunenko T, Zaneveld J, Knight R. QIIME allows analysis of high-throughput community sequencing data. Nat Methods 2010; 7: 335-336.
14. Haas B J, Gevers D, Earl A M, Feldgarden M, Ward D V, Giannoukos G, Ciulla D, Tabbaa D, Highlander S K, Sodergren E, Methe B, DeSantis T Z, Consortium H M, Petrosino J F, Knight R, Birren B W. Chimeric 16S rRNA sequence formation and detection in Sanger and 454-pyrosequenced PCR amplicons. Genome Res 2011; 21: 494-504.
15. R Development Core Team. R: A language and environment for statistical computing. R Foundation for Statistical Computing 2008.
16. DeSantis T Z, Hugenholtz P, Larsen N, Rojas M, Brodie E L, Keller K, Huber T, Dalevi D, Hu P, Andersen G L. Greengenes, a chimera-checked 16S rRNA gene database and workbench compatible with ARB. Appl Environ Microbiol 2006; 72: 5069-5072.
17. Schmittgen T D, Livak K J. Analyzing real-time PCR data by the comparative C T method. Nat Protocols 2008; 3: 1101-1108.
18. Vazquez-Baeza Y, Pirrung M, Gonzalez A, Knight R. EMPeror: a tool for visualizing high-throughput microbial community data. Gigascience 2013; 2: 16.
19. Langille M G I, Zaneveld J, Caporaso J G, McDonald D, Knights D, Reyes J A, Clemente J C, Burkepile D E, Vega Thurber R L, Knight R, Beiko R G, Huttenhower C. Predictive functional profiling of microbial communities using 16S rRNA marker gene sequences. Nat Biotechnol 2013; 31: 814-821.
20. Bray J R, J. T. Curtis. An ordination of upland forest communities of southern Wisconsin. Ecological Monographs 1957; 27: 325-349.
21. Holmes I, Harris K, Quince C. Dirichlet multinomial mixtures: generative models for microbial metagenomics. PLoS One 2012; 7: e30126.
22. Anderson M J. A new method for non-parametric multivariate analysis of variance. Austral Ecology 2001; 26: 32-46.
23. Lozupone C, Hamady M, Knight R. UniFrac—an online tool for comparing microbial community diversity in a phylogenetic context. BMC Bioinformatics 2006; 7: 371.
24. Lozupone C, Knight R. UniFrac: a new phylogenetic method for comparing microbial communities. Appl Environ Microbiol 2005; 71: 8228-8235.25. Rutherford S T, Bassler B L. Bacterial quorum sensing: its role in virulence and possibilities for its control. Cold Spring Harb Per spect Med 2012; 2.
25. Rutherford S T, Bassler B L. Bacterial quorum sensing: its role in virulence and possibilities for its control. Cold Spring Harb Perspect Med 2012; 2.
26. Antonic V, Stojadinovic A, Zhang B, Izadjoo M J, Alavi M. Pseudomonas aeruginosa induces pigment production and enhances virulence in a white phenotypic variant of Staphylococcus aureus. Infect Drug Resist 2013; 6: 175-186.
27. Kanehisa M, Goto S. KEGG: kyoto encyclopedia of genes and genomes. Nucleic Acids Res 2000; 28: 27-30.
28. Kanehisa M, Goto S, Sato Y, Kawashima M, Furumichi M, Tanabe M. Data, information, knowledge and principle: back to metabolism in KEGG. Nucleic Acids Research 2014; 42: D199-D205.
29. Rahman A N C K, Mujib S, Fong I W, Ostrowski M A. TIM-3 and Its Immunoregulatory Role in HIV Infection. Journal of Clinical & Cellular Immunology 2012; 7: 007.
30. Jones R B, Ndhlovu L C, Barbour J D, Sheth P M, Jha A R, Long B R, Wong J C, Satkunarajah M, Schweneker M, Chapman J M, Gyenes G, Vali B, Hyrcza M D, Yue F Y, Kovacs C, Sassi A, Loutfy M, Halpenny R, Persad D, Spotts G, Hecht F M, Chun T-W, McCune J M, Kaul R, Rini J M, Nixon D F, Ostrowski M A. Tim-3 expression defines a novel population of dysfunctional T cells with highly elevated frequencies in progressive HIV-1 infection. J Exp Med 2008; 205: 2763-2779.
31. Golden-Mason L, Palmer B E, Kassam N, Townshend-Bulson L, Livingston S, McMahon B J, Castelblanco N, Kuchroo V, Gretch D R, Rosen H R. Negative immune regulator Tim-3 is overexpressed on T cells in hepatitis C virus infection and its blockade rescues dysfunctional CD4+ and CD8+ T cells. J Virol 2009; 83: 9122-9130.
32. Muller S, Radic M. Citrullinated Autoantigens: From Diagnostic Markers to Pathogenetic Mechanisms. Clin Rev Allergy Immunol 2014.
32. Cha L, Berry C M, Nolan D, Castley A, Fernandez S, French M A. Interferon-alpha, immune activation and immune dysfunction in treated HIV infection. Clin Trans Immunol 2014;
33. Burhop K E, Selig W M, Malik A B. Monohydroxyeicosatetraenoic acids (5-HETE and 15-HETE) induce pulmonary vasoconstriction and edema. Circ Res 1988; 62: 687-698.
35. Mobraten K, Haugbro T, Karlstrom E, Kleiveland C R, Lea T. Activation of the bile acid receptor TGR5 enhances LPS-induced inflammatory responses in a human monocytic cell line. JRecept Signal Transduct Res 2014: 1-8.
36. Gower J C. Generalized procrustes analysis. Psychometrika 1975; 40: 33-51.
37. Mantel N. The detection of disease clustering and a generalized regression approach. Cancer Res 1967; 27: 209-220.
38. Cribbs S K, Uppal K, Li S, Jones D P, Huang L, Tipton L, Fitch A, Greenblatt R M, Kingsley L, Guidot D M, Ghedin E, Morris A. Correlation of the lung microbiota with metabolic profiles in bronchoalveolar lavage fluid in HIV infection. Microbiome 2016; 4: 1-11.
39. Segal L N, Clemente J C, Tsay J-C J, Koralov S B, Keller B C, Wu B G, Li Y, Shen N, Ghedin E, Morris A, Diaz P, Huang L, Wikoff W R, Ubeda C, Artacho A, Rom W N, Sterman D H, Collman R G, Blaser M J, Weiden M D. Enrichment of the lung microbiome with oral taxa is associated with lung inflammation of a Th17 phenotype. Nature Microbiology 2016; 1: 16031.
40. Kolwijck E, van de Veerdonk F L. The potential impact of the pulmonary microbiome on immunopathogenesis of Aspergillus-related lung disease. Eur J Immunol 2014; 44: 31563165.
41. Cui L, Lucht L, Tipton L, Rogers M B, Fitch A, Kessinger C, Camp D, Kingsley L, Leo N, Greenblatt R M, Fong S, Stone S, Dermand J C, Kleerup E C, Huang L, Morris A, Ghedin E. Topographic diversity of the respiratory tract mycobiome and alteration in HIV and lung disease. Am J Respir Crit Care Med 2015; 191: 932-942.
42. Homma T, Kato A, Bhushan B, Norton J E, Suh L A, Carter R G, Gupta D S, Schleimer R P. Aspergillus fumigatus Activates PAR-2 and Skews Toward a Th2 Bias in Airway Epithelial Cells. Am J Respir Cell Mol Biol 2015.
43. Urb M, Snarr B D, Wojewodka G, Lehoux M, Lee M J, Ralph B, Divangahi M, King I L, McGovern T K, Martin J G, Fraser R, Radzioch D, Sheppard D C. Evolution of the Immune Response to Chronic Airway Colonization with Aspergillus fumigatus Hyphae. Infect Immun 2015; 83: 3590-3600.
42. Sada-Ovalle I, Chavez-Galan L, Torre-Bouscoulet L, Nava-Gamirio L, Barrera L, Jayaraman P, Torres-Rojas M, Salazar-Lezama M A, Behar S M. The Tim3-galectin 9 pathway induces antibacterial activity in human macrophages infected with Mycobacterium tuberculosis. J Immunol 2012; 189: 5896-5902.
45. Kilsgird O, Andersson P, Malmsten M, Nordin S L, Linge H M, Eliasson M, Sorenson E, Erjefalt J S, Bylund J, Olin A I, Sorensen O E, Egesten A. Peptidylarginine deiminases present in the airways during tobacco smoking and inflammation can citrullinate the host defense peptide LL-37, resulting in altered activities. Am J Respir Cell Mol Biol 2012; 46: 240-248.

What is claimed is:

1. A method of detecting an airway microbiome in a subject who has or is suspected of having a lung infection, the method comprising detecting bacteria, or a proportion of bacteria, in a biological sample from the subject that are in the family Prevotellaceae, Pseudomonadaceae, Sphingomonadaceae, Streptococcaceae, and/or Veillonellaceae.

2. The method of claim 1, further comprising detecting the diversity of microorganisms in the biological sample.

3. The method of claim 1, wherein detecting the airway microbiome in the biological sample comprises amplifying and sequencing 16S rRNA genes of microorganisms in the sample.

4. The method of claim 1, wherein detecting the airway microbiome in the biological sample comprises amplifying and sequencing the V4 region of 16S rRNA genes of microorganisms in the sample.

5. The method of claim 1, wherein detecting the airway microbiome in the biological sample comprises determining the number of families, the number of genera, the number of species, the Faith's Phylogenetic Diversity, the Shannon Diversity, and/or the Simpson Diversity of the microorganisms.

6. The method of claim 1, wherein the airway microbiome is a lung microbiome.

7. The method of claim 1, wherein detecting the airway microbiome in the biological sample comprises detecting whether
(a) the biological sample has an increased proportion of bacteria in the Pseudomonadaceae family of bacteria compared to a general population of subjects who have pneumonia and are infected with HIV;
(b) the biological sample has an increased proportion of bacteria in the Pseudomonadaceae family of bacteria compared to a general population of subjects who have pneumonia and are infected with HIV, wherein the increased proportion is a proportion of at least about 10%, 20%, 30%, 40%, or 50%;
(c) at least 40% of the bacteria in the biological sample are in the Pseudomonadaceae family of bacteria, at least 10% of the bacteria in the biological sample are in the Sphingomonadaceae family of bacteria, and at least 5% of the bacteria in the biological sample are in the Prevotellaceae family of bacteria;
(d) the biological sample has an increased proportion of bacteria in the Streptococcaceae family of bacteria compared to a general population of subjects who have pneumonia and are infected with HIV;
(e) the biological sample has an increased proportion of bacteria in the Streptococcaceae family of bacteria compared to a general population of subjects who have pneumonia and are infected with HIV, wherein the increased proportion is a proportion of at least about 10%, 20%, 30%, 40%, or 50%;
(f) at least 40% of the bacteria in the biological sample are in the Streptococcaceae family of bacteria, at least 5% of the bacteria in the biological sample are in the Veillonellaceae family of bacteria, and at least 10% of the bacteria in the biological sample are in the Prevotellaceae family of bacteria;
(g) the biological sample has an increased proportion of bacteria in the Prevotellaceae family of bacteria compared to a general population of subjects who have pneumonia and are infected with HIV;
(h) the biological sample has an increased proportion of bacteria in the Prevotellaceae family of bacteria compared to a general population of subjects who have pneumonia and are infected with HIV, wherein the increased proportion is a proportion of at least about 10%, 20%, 30%, 40%, or 50%; and/or
(i) at least 30% of the bacteria in the biological sample are in the Prevotellaceae family of bacteria, at least 10% of the bacteria in the biological sample are in the Streptococcaceae family of bacteria, and at least 10% of the bacteria in the biological sample are in the Veillonellaceae family of bacteria.

8. The method of claim 1, wherein detecting the airway microbiome in the biological sample comprises detecting whether the subject
(a) has an increased proportion of lung microbiome bacteria in the Pseudomonadaceae family of bacteria compared to a healthy or general population of subjects;
(b) has an increased proportion of lung microbiome bacteria in the Pseudomonadaceae family of bacteria compared to a healthy or general population of subjects, wherein the increased proportion is a proportion of at least about 10%, 20%, 30%, 40%, or 50%; and/or
(c) has a proportion of at least 40% of lung microbiome bacteria in the Pseudomonadaceae family of bacteria, at least 10% of lung microbiome bacteria in the Sphingomonadaceae family of bacteria, and at least 5% of lung microbiome bacteria in the Prevotellaceae family of bacteria.

9. The method of claim 1, wherein detecting the airway microbiome in the biological sample comprises detecting whether the subject
(a) has an increased proportion of lung microbiome bacteria in the Pseudomonadaceae family of bacteria compared to a healthy or general population of subjects;
(b) has an increased proportion of lung microbiome bacteria in the Pseudomonadaceae family of bacteria compared to a healthy or general population of subjects, wherein the increased proportion is a proportion of at least about 10%, 20%, 30%, 40%, or 50%; and/or
(c) has a proportion of at least 40% of lung microbiome bacteria in the Pseudomonadaceae family of bacteria, at least 10% of lung microbiome bacteria in the Sphingomonadaceae family of bacteria, and at least 5% of lung microbiome bacteria in the Prevotellaceae family of bacteria.

10. The method of claim 1, wherein detecting the airway microbiome in the biological sample comprises detecting whether the subject
(a) has an increased proportion of lung microbiome bacteria in the Prevotellaceae family of bacteria compared to a healthy or general population of subjects;
(b) has an increased proportion of lung microbiome bacteria in the Prevotellaceae family of bacteria compared to a healthy or general population of subjects, wherein the increased proportion is a proportion of at least about 10%, 20%, 30%, 40%, or 50%; and/or
(c) has a proportion of at least 30% of lung microbiome bacteria in the Prevotellaceae family of bacteria, at least 10% lung microbiome bacteria in the Streptococcaceae family of bacteria, and at least 10% lung microbiome bacteria in the Veillonellaceae family of bacteria.

11. The method of claim 1, wherein detecting the airway microbiome in the biological sample comprises detecting whether the subject
(a) has an increased proportion of lung microbiome bacteria in the Prevotellaceae family of bacteria compared to the general population;
(b) has an increased proportion of lung microbiome bacteria in the Prevotellaceae family of bacteria compared to the general population, wherein the increased proportion is a proportion of at least about 10%, 20%, 30%, 40%, or 50%; and/or
(c) has a proportion of at least 30% of lung microbiome bacteria in the Prevotellaceae family of bacteria, at least 10% of lung microbiome bacteria in the Streptococcaceae family of bacteria, and at least 10% of lung microbiome bacteria in the Veillonellaceae family of bacteria.

12. A method of detecting at least one immune protein or immune protein-encoding mRNA in a subject who has or is suspected of having a lung infection, the method comprising detecting the level of TIM-3 protein or TIM-3 mRNA in a biological sample from the subject.

13. The method of claim 12, comprising determining whether the level TIM-3 protein or TIM-3 mRNA is increased compared to a general or healthy population of subjects.

14. The method of claim 12, further comprising detecting the level of any one of or any combination of 2, 3, 4, 5, 6, 7, 8, 9, 10, 11, 12, 13, 14, 15, 16, 17, 18, or 19 of
(i) IFNγ protein, IFNα protein, TNFα protein, MUC5AC protein, IL-17A protein, IL-4 protein, IL-5 protein, IL-13 protein, IL-33 protein, OCEL1 protein, CCL11 protein, PADI4 protein, IL-10 protein, FOXP3 protein, PD-1 protein, CD45RO protein, CD2 protein, CD39 protein, and GAPDH protein; or
(ii) IFNγ mRNA, IFNα mRNA, TNFα mRNA, MUC5AC mRNA, IL-17A mRNA, IL-4 mRNA, IL-5 mRNA, IL-13 mRNA, IL-33 mRNA, OCEL1 mRNA, CCL11 mRNA, PADI4 mRNA, IL-10 mRNA, FOXP3 mRNA, PD-1 mRNA, CD45RO mRNA, CD2 mRNA, CD39 mRNA, and GAPDH mRNA,
in the biological sample.

15. The method of claim 12, wherein no more than 1000, 900, 800, 700, 600, 500, 400, 300, 200, 100, 50, 20, or 10 proteins or mRNAs are detected.

16. The method of claim 12, wherein the detecting does not comprise the use of a microarray.

17. The method of claim 12, wherein the detecting comprises the use of a microarray that detects the mRNA levels of less than 1000, 900, 800, 700, 600, 500, 400, 300, 200, 100, 50, 20, or 10 genes.

18. The method of claim 12, comprising detecting whether the subject has an increased level of expression of 1 of or any combination of 2, 3, 4, 5, 6, 7, 8, 9, 10, 11, 12, 13, 14, 15, or 16 of IFNγ, IFNα, TNFα, MUC5AC, IL-17A, IL-4, IL-5, IL-13, IL-33, OCEL1, CCL11, FOXP3, PD-1, CD45RO, CD39, and/or GAPDH compared to a general or healthy population.

19. The method of claim 12, comprising detecting whether the subject has an increased level of expression of 1 of or any combination of 2, 3, 4, 5, 6, 7, 8, 9, 10, 11, 12, 13, 14, 15, or 16 of IFNγ, IFNα, TNFα, MUC5AC, IL-17A, IL-4, IL-5, IL-13, IL-33, OCEL1, CCL11, FOXP3, PD-1, CD45RO, CD39, and/or GAPDH compared to the general population;

20. The method of claim 1, wherein the biological sample is from an airway of the subject.

21. The method of claim 1, wherein the airway is in a lung of the subject.

22. The method of claim 1, wherein the airway is a trachea, bronchus, bronchiole, alveolar duct, alveolar sac, and/or alveolus.

23. The method of claim 1, wherein the biological sample is a bronchoalveolar lavage (BAL) sample, sputum, phlegm, saliva, or mucus.

24. A method of detecting at least one metabolite in a subject who has or is suspected of having a lung infection, the method comprising detecting the at least one metabolite in a biological sample from the subject, wherein the at least one metabolite is a tryptophan metabolite, an arachidonic acid metabolite, an eicosanoid, or a product of primary or secondary bile metabolism.

25. The method of claim 24, wherein the biological sample is a bodily fluid.

26. The method of claim 25, wherein the bodily fluid is blood, serum, or plasma.

27. The method of claim 24, wherein the at least one metabolite is leukotriene B4, xanthurenate, 15-hydroxyeicosatetraenoic acid, chenodeoxycholate, glycodeoxycholate, taurochenodeoxycholate, or ursodeoxycholate.

28. The method of claim 24, wherein the at least one metabolite is a lysolipid metabolite, a pyrimidine metabolite, or a monoacylglycerol.

29. The method of claim 24, wherein the at least one metabolite is a valine metabolite, a leucine metabolite, or a monoacylglycerol associated with lipid metabolism.

30. The method of claim 24, wherein the at least one metabolite is 3-methyl-2-oxobutyrate, 4-methyl-2-oxopentanoate, 1-dihomo-linolenylglycerol, 1-myristoylglycerol, or inosine.

31. The method of claim 24, comprising detecting whether the subject has an increased level of 1, 2, 3, 4, 5, 6, or 7 of leukotriene B4, xanthurenate, 15-hydroxyeicosatetraenoic acid, chenodeoxycholate, glycodeoxycholate, taurochenodeoxycholate, and/or ursodeoxycholate compared to a general or healthy population of subjects.

32. The method of claim 24, comprising detecting whether the subject has an increased level of 1, 2, 3, 4, 5, 6, or 7 of leukotriene B4, xanthurenate, 15-hydroxyeicosatetraenoic acid, chenodeoxycholate, glycodeoxycholate, taurochenodeoxycholate, and/or ursodeoxycholate compared to a general or healthy population of subjects, or has an increased level of 1, 2, 3, or 5 or 3-methyl-2-oxobutyrate, 4-methyl-2-oxopentanoate, 1-dihomo-linolenylglycerol, 1-myristoylglycerol, or inosine compared to a general or healthy population of subjects.

33. The method of claim 24, comprising detecting whether the subject has an increased level of 1, 2, 3, or 5 or 3-methyl-2-oxobutyrate, 4-methyl-2-oxopentanoate, 1-dihomo-linolenylglycerol, 1-myristoylglycerol, or inosine compared to the general population.

34. The method of claim 1, wherein the subject is infected with human immunodeficiency virus (HIV) or is suspected of being infected with HIV.

35. The method of claim 1, wherein the subject has or is suspected of having pneumonia.

36. The method of claim 35, wherein the pneumonia is bacterial pneumonia or fungal pneumonia.

37. The method of claim 1, wherein the subject has or is suspected of having tuberculosis (TB).

38. The method of claim 1, wherein the subject has or is suspected of having TB pneumonia.

39. The method of claim 1, wherein the subject has been administered an antibiotic.

40. The method of claim 39, wherein the antibiotic is ceftriaxone.

41. A method of treating or preventing a Mycobacterium sp. infection in a subject in need thereof, the method comprising administering an effective amount of at least one antibiotic compound to the subject, wherein the subject
(a) has increased TIM-3 expression compared to a general or healthy population of subjects;
(b) has an increased level of 1, 2, 3, 4, 5, 6, or 7 of leukotriene B4, xanthurenate, 15-hydroxyeicosatetraenoic acid, chenodeoxycholate, glycodeoxycholate,
taurochenodeoxycholate, and/or ursodeoxycholate compared to a general or healthy population of subjects;
(c) has an increased proportion of lung microbiome bacteria in the Pseudomonadaceae family of bacteria compared to a healthy or general population of subjects;
(d) has an increased proportion of lung microbiome bacteria in the Pseudomonadaceae family of bacteria compared to a healthy or general population of subjects, wherein the increased proportion is a proportion of at least about 10%, 20%, 30%, 40%, or 50%; and/or
(e) has a proportion of at least 40% of lung microbiome bacteria in the Pseudomonadaceae family of bacteria, at least 10% of lung microbiome bacteria in the Sphingomonadaceae family of bacteria, and at least 5% of lung microbiome bacteria in the Prevotellaceae family of bacteria.

42. A method of treating or preventing a Mycobacterium sp. infection in a subject in need thereof, the method comprising:
(a) detecting (i) an airway microbiome; (ii) a diversity of microorganisms; (iii) a plurality of microorganisms; and/or (iv) at least one metabolite, in a biological sample from the subject; and
(b) administering to the subject an effective amount of at least one antibiotic compound.

43. The method of claim 41, wherein the at least one antibiotic compound is 1, 2, 3, or 4 of any combination of isoniazid, rifampin, ethambutol, and/or pyrazinamide.

44. The method of claim 41, wherein the at least one antibiotic compound is isoniazid, rifampin, and pyrazinamide.

45. The method of claim 41, wherein the at least one antibiotic compound is isoniazid and rifampin.

46. The method of claim 41, wherein the at least one antibiotic compound is an aminoglycoside, a fluoroquinolone, a polypeptide, a thioamide, a cycloserine, and/or p-aminosalicylic acid.

47. A method of treating or preventing an Aspergillus sp. infection in a subject in need thereof, the method comprising administering an effective amount of at least one antifungal agent to the subject, wherein the subject
(a) has an increased level of expression of 1 of or any combination of 2, 3, 4, 5, 6, 7, 8, 9, 10, 11, 12, 13, 14, 15, or 16 of IFNγ, IFNα, TNFα, MUC5AC, IL-17A, IL-4, IL-5, IL-13, IL-33, OCEL1, CCL11, FOXP3, PD-1, CD45RO, CD39, and/or GAPDH compared to a general or healthy population;
(b) has an increased level of 1, 2, 3, or 5 or 3-methyl-2-oxobutyrate, 4-methyl-2-oxopentanoate, 1-dihomo-linolenylglycerol, 1-myristoylglycerol, or inosine compared to a general or healthy population of subjects;
(c) has an increased proportion of lung microbiome bacteria in the Prevotellaceae family of bacteria compared to a healthy or general population of subjects;
(d) has an increased proportion of lung microbiome bacteria in the Prevotellaceae family of bacteria compared to a healthy or general population of subjects, wherein the increased proportion is a proportion of at least about 10%, 20%, 30%, 40%, or 50%; and/or
(e) has a proportion of at least 30% of lung microbiome bacteria in the Prevotellaceae family of bacteria, at least 10% lung microbiome bacteria in the Streptococcaceae family of bacteria, and at least 10% lung microbiome bacteria in the Veillonellaceae family of bacteria.

48. A method of treating or preventing an Aspergillus sp. infection in a subject in need thereof, the method comprising:
(a) detecting (i) an airway microbiome; (ii) the diversity of microorganisms; (iii) a plurality of microorganisms; and/or (iv) at least one metabolite, in a biological sample from the subject; and
(b) administering to the subject an effective amount of at least one antifungal agent.

49. The method of claim 47, wherein the at least one antifungal agent is a triazole antifungal agent.

50. The method of claim 47, wherein the at least one antifungal agent is 1, 2, 3, 4, 5, 6, or 7 of any combination of amphotericin B, liposomal amphotericin B, voriconazole, caspofungin, flucytosine, itraconazole, and/or posaconazole.

51. The method of claim 41, wherein the Mycobacterium sp. is M. tuberculosis.

52. The method of claim 47, wherein the Aspergillus sp. is A. fumigatus or A. flavus.

53. A method of treating or preventing a lung infection in a subject in need thereof, the method comprising administering an effective amount of at least one antibiotic or antifungal agent to the subject, wherein the subject
(a) has an increased level of expression of 1 of or any combination of 2, 3, 4, 5, 6, 7, 8, 9, 10, 11, 12, 13, 14, 15, or 16 of IFNγ, IFNα, TNFα, MUC5AC, IL-17A, IL-4, IL-5, IL-13, IL-33, OCEL1, CCL11, FOXP3, PD-1, CD45RO, CD39, and/or GAPDH compared to a general or healthy population;
(b) has an increased level of 1, 2, 3, or 5 or 3-methyl-2-oxobutyrate, 4-methyl-2-oxopentanoate, 1-dihomo-linolenylglycerol, 1-myristoylglycerol, or inosine compared to a general or healthy population of subjects;
(c) has an increased proportion of lung microbiome bacteria in the Prevotellaceae family of bacteria compared to a healthy or general population of subjects;
(d) has an increased proportion of lung microbiome bacteria in the Prevotellaceae family of bacteria compared to a healthy or general population of subjects, wherein the increased proportion is a proportion of at least about 10%, 20%, 30%, 40%, or 50%;
(e) has a proportion of at least 30% of lung microbiome bacteria in the Prevotellaceae family of bacteria, at least 10% lung microbiome bacteria in the Streptococcaceae family of bacteria, and at least 10% lung microbiome bacteria in the Veillonellaceae family of bacteria;
(f) has increased TIM-3 expression compared to a general or healthy population of subjects;
(g) has an increased level of 1, 2, 3, 4, 5, 6, or 7 of leukotriene B4, xanthurenate, 15-hydroxyeicosatetraenoic acid, chenodeoxycholate, glycodeoxycholate, taurochenodeoxycholate, and/or ursodeoxycholate compared to a general or healthy population of subjects;
(h) has an increased proportion of lung microbiome bacteria in the Pseudomonadaceae family of bacteria compared to a healthy or general population of subjects;
(i) has an increased proportion of lung microbiome bacteria in the Pseudomonadaceae family of bacteria compared to a healthy or general population of subjects, wherein the increased proportion is a proportion of at least about 10%, 20%, 30%, 40%, or 50%; and/or
(j) has a proportion of at least 40% of lung microbiome bacteria in the Pseudomonadaceae family of bacteria, at least 10% of lung microbiome bacteria in the Sphingomonadaceae family of bacteria, and at least 5% of lung microbiome bacteria in the Prevotellaceae family of bacteria.

54. A method of treating or preventing a lung infection in a subject in need thereof, the method comprising:
(a) detecting (i) an airway microbiome; (ii) the diversity of microorganisms; (iii) a plurality of microorganisms; and/or (iv) at least one metabolite, in a biological sample from the subject; and
(b) administering to the subject an effective amount of at least one antibiotic or antifungal agent.

55. The method of claim 41, wherein the subject is infected with human immunodeficiency virus (HIV) or is suspected of being infected with HIV.

56. The method of claim 41, wherein the subject has pneumonia.

57. The method of claim 56, wherein the pneumonia is bacterial pneumonia.

58. The method of claim 41, wherein the subject has tuberculosis (TB).

59. The method of claim 41, wherein the subject has TB pneumonia.

60. The method of claim 41, wherein the subject has been administered an antibiotic.

61. The method of claim 60, wherein the antibiotic is ceftriaxone.
